SEQUENCE LISTING (256 sequences)

SEQ ID NO: 1 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 2 - DNA, 22 bp, Artificial
  C1221 target
caaaacgtcgtacgacgttttg

SEQ ID NO: 3 - DNA, 22 bp, Artificial
  R1 target
tgttctcaggtacctcagccag

SEQ ID NO: 4 - DNA, 22 bp, Artificial
  D21 target
aaacctcaagtaccaaatgtaa

SEQ ID NO: 5 - DNA, 30 bp, Artificial
  Adaptor A Deep Sequencing Primer
  misc_feature (27)..(30): n = a, t, c, or g
ccatctcatccctgcgtgtctccgacnnnn

SEQ ID NO: 6 - DNA, 30 bp, Artificial
  Adaptor B Deep sequencing primer
cctatcccctgtgtgccttggcagtctcag

SEQ ID NO: 7 - protein, 3 aa, Artificial
  1a8h_1 peptidic linker
NVG

SEQ ID NO: 8 - protein, 4 aa, Artificial
  1dnpA_1 peptidic linker
DSVI

SEQ ID NO: 9 - protein, 4 aa, Artificial
  1d8cA_2 peptidic linker
IVEA

SEQ ID NO: 10 - protein, 4 aa, Artificial
  1ckqA_3 peptidic linker
LEGS

SEQ ID NO: 11 - protein, 4 aa, Artificial
  1sbp_1 peptidic linker
YTST

SEQ ID NO: 12 - protein, 5 aa, Artificial
  1ev7A_1 peptidic linker
LQENL

SEQ ID NO: 13 - protein, 5 aa, Artificial
  1alo_3 peptidic linker
VGRQP

SEQ ID NO: 14 - protein, 5 aa, Artificial
  1amf_1 peptidic linker
LGNSL

SEQ ID NO: 15 - protein, 6 aa, Artificial
  1adjA_3 peptidic linker
LPEEKG

SEQ ID NO: 16 - protein, 6 aa, Artificial
  1fcdC_1 peptidic linker
QTYQPA

SEQ ID NO: 17 - protein, 6 aa, Artificial
  1al3_2 peptidic linker
FSHSTT

SEQ ID NO: 18 - protein, 7 aa, Artificial
  1g3p_1 peptidic linker
GYTYINP

SEQ ID NO: 19 - protein, 7 aa, Artificial
  1acc_3 peptidic linker
LTKYKSS

SEQ ID NO: 20 - protein, 8 aa, Artificial
  1ahjB_1 peptidic linker
SRPSESEG

SEQ ID NO: 21 - protein, 8 aa, Artificial
  1acc_1 peptidic linker
PELKQKSS

SEQ ID NO: 22 - protein, 8 aa, Artificial
  1af7_1 peptidic linker
LTTNLTAF

SEQ ID NO: 23 - protein, 9 aa, Artificial
  1heiA_1 peptidic linker
TATPPGSVT

SEQ ID NO: 24 - protein, 9 aa, Artificial
  1bia_2 peptidic linker
LDNFINRPV

SEQ ID NO: 25 - protein, 9 aa, Artificial
  1igtB_1 peptidic linker
VSSAKTTAP

SEQ ID NO: 26 - protein, 10 aa, Artificial
  1nfkA_1 peptidic linker
DSKAPNASNL

SEQ ID NO: 27 - protein, 10 aa, Artificial
  1au7A_1 peptidic linker
KRRTTISIAA

SEQ ID NO: 28 - protein, 11 aa, Artificial
  1bpoB_1 peptidic linker
PVKMFDRHSSL

SEQ ID NO: 29 - protein, 11 aa, Artificial
  1b0pA_2 peptidic linker
APAETKAEPMT

SEQ ID NO: 30 - protein, 14 aa, Artificial
  1c05A_2 peptidic linker
YTRLPERSELPAEI

SEQ ID NO: 31 - protein, 14 aa, Artificial
  1gcb_1 peptidic linker
VSTDSTPVTNQKSS

SEQ ID NO: 32 - protein, 14 aa, Artificial
  1bt3A_1 peptidic linker
YKLPAVTTMKVRPA

SEQ ID NO: 33 - protein, 15 aa, Artificial
  1b3oB_2 peptidic linker
IARTDLKKNRDYPLA

SEQ ID NO: 34 - protein, 21 aa, Artificial
  16vpA_6 peptidic linker
TEEPGAPLTTPPTLHGNQARA

SEQ ID NO: 35 - protein, 21 aa, Artificial
  1dhx_1 peptidic linker
ARFTLAVGDNRVLDMASTYFD

SEQ ID NO: 36 - protein, 26 aa, Artificial
  1b8aA_1 peptidic linker
IVVLNRAETPLPLDPTGKVKAELDTR

SEQ ID NO: 37 - protein, 28 aa, Artificial
  1qu6A_1 peptidic linker
ILNKEKKAVSPLLLTTTNSSEGLSMGNY

SEQ ID NO: 38 - protein, 158 aa, Methylophilus methylotrophus
  GenBank ACC85607.1 residues 2 to 159
ALSWNEIRRKAIEFSKRWEDASDENSQAKPFLIDFFEVFGITNKRVATFEHAVKKFAKAHKEQSRGFVDLFWPGILLIEMKSRGKDLDKAYDQALDYFSGIAERDLPRYVLVCDFQRFRLTDLITKESVEFLLKDLYQNVRSFGFIAGYQTQVIKPQD

SEQ ID NO: 39 - protein, 155 aa, Artificial
  EsaSSI, GenBank EAJ03172.1 residues 2 to 156
AALSFPEIRTRLQAFAKQWKQAERENADAKLFWARFYECFGIRPESATIYEKAVDKLDGSRGFIDSFIPGLLIVEHKSKGKDLNSAFTQASDYFTALAEGERPRYIIVSDFARFRLYDLKTDTQVECKLADISKHAGWFRFLVEGEATPEIVEES

SEQ ID NO: 40 - protein, 179 aa, Corynebacterium striatum
  NCBI Reference Sequence NP_862240 residues 2 to 180
VMAPTTVFDRATIRHNLTEFKLRWLDRIKQWEAENRPATESSHDQQFWGDLLDCFGVNARDLYLYQRSAKRASTGRTGKIDMFMPGKVIGEAKSLGVPLDDAYAQALDYLLGGTIANSHMPAYVVCSNFETLRVTRLNRTYVGDSADWDITFPLAEIDEHIEQLAFLADYETSAYREEE

SEQ ID NO: 41 - protein, 250 aa, Nostoc sp. PCC 7120 (Anabaena sp. PCC 7120)
  GenBank CAA45962.1 residues 25 to 274
QVPPLTELSPSISVHLLLGNPSGATPTKLTPDNYLMVKNQYALSYNNSKGTANWVAWQLNSSWLGNAERQDNFRPDKTLPAGWVRVTPSMYSGSGYDRGHIAPSADRTKTTEDNAATFLMTNMMPQTPDNNRNTWGNLEDYCRELVSQGKELYIVAGPNGSLGKPLKGKVTVPKSTWKIVVVLDSPGSGLEGITANTRVIAVNIPNDPELNNDWRAYKVSVDELESLTGYDFLSNVSPNIQTSIESKVDN

SEQ ID NO: 42 - protein, 213 aa, Escherichia coli (strain K12)
  UniProtKB/Swiss Prot P25736, residues 23 to 235
EGINSFSQAKAAAVKVHADAPGTFYCGCKINWQGKKGVVDLQSCGYQVRKNENRASRVEWEHVVPAWQFGHQRQCWQDGGRKNCAKDPVYRKMESDMHNLQPSVGEVNGDRGNFMYSQWNGGEGQYGQCAMKVDFKEKAAEPPARARGAIARTYFYMRDQYNLTLSRQQTQLFNAWNKMYPVTDWECERDERIAKVQGNHNPYVQRACQARKS

SEQ ID NO: 43 - protein, 247 aa, Dickeya dadantii (strain 3937) (Erwinia chrysanthemi (strain 3937))
  UniProtKB/Swiss Prot P37994, residues 20 to 266
AAGQDINNFTQAKAAAAKIHQDAPGTFYCGCKINWQGKKGTPDLASCGYQVRKDANRASRIEWEHVVPAWQFGHQRQCWQDGGRKNCTKDDVYRQIETDLHNLQPAIGEVNGDRGNFMYSQWNGGERQYGQCEMKIDFKSQLAEPPERARGAIARTYFYMRDRYNLNLSRQQTQLFDAWNKQYPATTWECTREKRIAAVQGNHNPYVQQACSPDAAPYYNGLSLIMIAAVATVAARWLTPAGHLPSD

SEQ ID NO: 44 - protein, 250 aa, Streptococcus pneumoniae
  UniProtKB/Swiss Prot P0A3S3, DNA-entry nuclease, residues 25 to 274
IKQMPSAPNSPKTNLSQKKQASEAPSQALAESVLTDAVKSQIKGSLEWNGSGAFIVNGNKTNLDAKVSSKPYADNKTKTVGKETVPTVANALLSKATRQYKNRKETGNGSTSWTPPGWHQVKNLKGSYTHAVDRGHLLGYALIGGLDGFDASTSNPKNIAVQTAWANQAQAEYSTGQNYYESKVRKALDQNKRVRYRVTLYYASNEDLVPSASQIEAKSSDGELEFNVLVPNVQKGLQLDYRTGEVTVTQ

SEQ ID NO: 45 - protein, 149 aa, Staphylococcus aureus
  UniProtKB/Swiss Prot P00644, residues 83 to 231
ATSTKKLHKEPATLIKAIDGDTVKLMYKGQPMTFRLLLVDTPETKHPKKGVEKYGPEASAFTKKMVENAKKIEVEFDKGQRTDKYGRGLAYIYADGKMVNEALVRQGLAKVAYVYKPNNTHEQHLRKSEAQAKKEKLNIWSEDNADSGQ

SEQ ID NO: 46 - protein, 143 aa, Staphylococcus hyicus
  UniProtKB/Swiss Prot P43270, residues 27 to 169
GPFKSAGLSNANEQTYKVIRVIDGDTIIVDKDGKQQNLRMIGVDTPETVKPNTPVQPYGKEASDFTKRHLTNQKVRLEYDKQEKDRYGRTLAYVWLGKEMFNEKLAKEGLARAKFYRPNYKYQERIEQAQKQAQKLKKNIWSN

SEQ ID NO: 47 - protein, 151 aa, Shigella flexneri
  UniProtKB/Swiss Prot P29769, residues 24 to 174
WADFRGEVVRILDGDTIDVLVNRQTIRVRLADIDAPESGQAFGSRARQRLADLTFRQEVQVTEKEVDRYGRTLGVVYAPLQYPGGQTQLTNINAIMVQEGMAWAYRYYGKPTDAQMYEYEKEARRQRLGLWSDPNAQEPWKWRRASKNATN

SEQ ID NO: 48 - protein, 192 aa, Bacillus subtilis
  UniProtKB/Swiss Prot P94492, residues 20 to 211
CGSNHAAKNHSDSNGTEQVSQDTHSNEYNQTEQKAGTPHSKNQKKLVNVTLDRAIDGDTIKVIYNGKKDTVRYLLVDTPETKKPNSCVQPYGEDASKRNKELVNSGKLQLEFDKGDRRDKYGRLLAYVYVDGKSVQETLLKEGLARVAYVYEPNTKYIDQFRLDEQEAKSDKLSIWSKSGYVTNRGFNGCVK

SEQ ID NO: 49 - protein, 148 aa, Enterobacteria phage T7 (Bacteriophage T7)
  UniProtKB/Swiss Prot P00641, residues 2 to 149
AGYGAKGIRKVGAFRSGLEDKVSKQLESKGIKFEYEEWKVPYVIPASNHTYTPDFLLPNGIFVETKGLWESDDRKKHLLIREQHPELDIRIVFSSSRTKLYKGSPTSYGEFCEKHGIKFADKLIPAEWIKEPKKEVPFDRLKRKGGKK

SEQ ID NO: 50 - protein, 251 aa, Bos taurus
  UniProtKB/Swiss Prot, P38447, residues 49 to 299
AGLPAVPGAPAGGGPGELAKYGLPGVAQLKSRASYVLCYDPRTRGALWVVEQLRPEGLRGDGNRSSCDFHEDDSVHAYHRATNADYRGSGFDRGHLAAAANHRWSQKAMDDTFYLSNVAPQVPHLNQNAWNNLEKYSRSLTRTYQNVYVCTGPLFLPRTEADGKSYVKYQVIGKNHVAVPTHFFKVLILEAAGGQIELRSYVMPNAPVDEAIPLEHFLVPIESIERASGLLFVPNILARAGSLKAITAGSK

SEQ ID NO: 51 - protein, 129 aa, Thermus thermophilus (strain HB8 / ATCC 27634 / DSM 579)
  UniProtKB/Swiss Prot, Q56239, residues 2 to 130 DNA mismatch repair protein mutS
GGYGGVKMEGMLKGEGPGPLPPLLQQYVELRDRYPDYLLLFQVGDFYECFGEDAERLARALGLVLTHKTSKDFTTPMAGIPIRAFDAYAERLLKMGFRLAVADQVEPAEEAEGLVRREVTQLLTPGTLT

SEQ ID NO: 52 - protein, 239 aa, Homo sapiens
  UniProtKB/Swiss Prot, Q53H47, residues 433 to 671 Histone-lysine N-methyltransferase SETMAR
HLKQIGKVKKLDKWVPHELTENQKNRRFEVSSSLILRNHNEPFLDRIVTCDEKWILYDNRRRSAQWLDQEEAPKHFPKPILHPKKVMVTIWWSAAGLIHYSFLNPGETITSEKYAQEIDEMNQKLQRLQLALVNRKGPILLHDNARPHVAQPTLQKLNELGYEVLPHPPYSPDLLPTNYHVFKHLNNFLQGKRFHNQQDAENAFQEFVESQSTDFYATGINQLISRWQKCVDCNGSYFD

SEQ ID NO: 53 - protein, 213 aa, Vibrio vulnificus
  GenBank AAF19759.1 residues 18 to 231
APPSSFSAAKQQAVKIYQDHPISFYCGCDIEWQGKKGIPNLETCGYQVRKQQTRASRIEWEHVVPAWQFGHHRQCWQKGGRKNCSKNDQQFRLMEADLHNLTPAIGEVNGDRSNFNFSQWNGVDGVSYGRCEMQVNFKQRKVMPPDRARGSIARTYLYMSQEYGFQLSKQQQQLMQAWNKSYPVDEWECTRDDRIAKIQGNHNPFVQQSCQTQ

SEQ ID NO: 54 - protein, 131 aa, Escherichia coli
  UniProtKB/Swiss Prot, Q47112, residues 446 to 576
KRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGK

SEQ ID NO: 55 - protein, 174 aa, Bacillus phage SP01 (Bacteriophage SP01)
  UniProtKB/Swiss Prot, P34081, DNA endonuclease I-HmuI residues 1 to 174
MEWKDIKGYEGHYQVSNTGEVYSIKSGKTLKHQIPKDGYHRIGLFKGGKGKTFQVHRLVAIHFCEGYEEGLVVDHKDGNKDNNLSTNLRWVTQKINVENQMSRGTLNVSKAQQIAKIKNQKPIIVISPDGIEKEYPSTKCACEELGLTRGKVTDVLKGHRIHHKGYTFRYKLNG

SEQ ID NO: 56 - protein, 169 aa, Enterobacteria phage T4 (Bacteriophage T4)
  UniProtKB/Swiss Prot, P13299, residues 2 to 170
KSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNR

SEQ ID NO: 57 - protein, 145 aa, Enterobacteria phage RB3 (Bacteriophage RB3)
  UniProtKB/Swiss Prot, Q38419, residues 2 to 146
NYRKIWIDANGPIPKDSDGRTDEIHHKDGNRENNDLDNLMCLSIQEHYDIHLAQKDYQACHAIKLRMKYSPEEISELASKAAKSREIQIFNIPEVRAKNIASIKSKIENGTFHLLDGEIQRKSNLNRVALGIHNFQQAEHIAKVK

SEQ ID NO: 58 - protein, 354 aa, Artificial
  R1 single chain meganuclease
MANTKYNEEFLLYLAGFVDGDGSIIAQINPNQSSKFKHRLRLTFYVTQKTQRRWFLDKLVDEIGVGYVRDSGSVSQYVLSEIKPLHNFLTQLQPFLELKQKQANLVLKIIEQLPSAKESPDKFLEVCTWVDQIAALNDSKTRKTTSETVRAVLDSLSGKKKSSPAAGGSDKYNQALSKYNQALSKYNQALSGGGGSNKKFLLYLAGFVDSDGSIIAQIKPRQSNKFKHQLSLTFAVTQKTQRRWFLDKLVDRIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 59 - protein, 354 aa, Artificial
  D21 single chain meganuclease
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGYVRDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 60 - protein, 245 aa, Artificial
  I-TevI
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISEKMKGKKPSNIKKISCDGVIFDCAADAARHFKISSGLVTYRVKSDKWNWFYINA

SEQ ID NO: 61 - protein, 366 aa, Artificial
  hTevCre_D01
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISGKMKGQGPSGNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 62 - protein, 353 aa, Artificial
  hTevCre_D02
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSQGPSGNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 63 - protein, 339 aa, Artificial
  hTevCre_D03
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNQGPSGNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 64 - protein, 318 aa, Artificial
  hTevCre_D04
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHQGPSGNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 65 - protein, 293 aa, Artificial
  hTevCre_D05
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKQGPSGNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 66 - protein, 296 aa, Artificial
  hTevCre_D06
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKANTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 67 - protein, 5 aa, Artificial
  QGPSG peptidic linker
QGPSG

SEQ ID NO: 68 - protein, 8 aa, Artificial
  LGPDGRKA peptidic linker
LGPDGRKA

SEQ ID NO: 69 - protein, 167 aa, Artificial
  I-CreI_N20
MANTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 70 - protein, 366 aa, Artificial
  hTevCre_D01_N20
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSDITKSKISGKMKGQGPSGNTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 71 - protein, 353 aa, Artificial
  hTevCre_D02_N20
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNRSGENNSFFNHKHSQGPSGNTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 72 - protein, 339 aa, Artificial
  hTevCre_D03_N20
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNQGPSGNTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 73 - protein, 318 aa, Artificial
  hTevCre_D04_N20
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHQGPSGNTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 74 - protein, 293 aa, Artificial
  hTevCre_D05_N20
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKQGPSGNTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 75 - protein, 296 aa, Artificial
  hTevCre_D06_N20
MKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKANTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 76 - protein, 167 aa, Artificial
  I-CreI
MANTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 77 - protein, 157 aa, Artificial
  I-CreI_X
MANTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSAD

SEQ ID NO: 78 - protein, 20 aa, Artificial
  NFS1 peptidic linker
GSDITKSKISEKMKGQGPSG

SEQ ID NO: 79 - protein, 23 aa, Artificial
  NFS2 peptidic linker
GSDITKSKISEKMKGLGPDGRKA

SEQ ID NO: 80 - protein, 10 aa, Artificial
  CFS1 peptidic linker
SLTKSKISGS

SEQ ID NO: 81 - protein, 177 aa, Artificial
  I-CreI_NFS1
MAGSDITKSKISEKMKGQGPSGNTKYNKEFLLYLAGFVDGDGSIIAQIKPNQSYKFKHQLSLTFQVTQKTQRRWFLDKLVDEIGVGYVRDRGSVSDYILSEIAPLHNFLTQLQPFLKLKQKQANLVLKIIEQLPSAKESPDKFLEVCTWVDQIAALNDSKTRKTTSETVRAVLDSAD

SEQ ID NO: 82 - protein, 180 aa, Artificial
  I-CreI_NFS2
MAGSDITKSKISEKMKGLGPDGRKANTKYNKEFLLYLAGFVDGDGSIIAQIKPNQSYKFKHQLSLTFQVTQKTQRRWFLDKLVDEIGVGYVRDRGSVSDYILSEIAPLHNFLTQLQPFLKLKQKQANLVLKIIEQLPSAKESPDKFLEVCTWVDQIAALNDSKTRKTTSETVRAVLDSAD

SEQ ID NO: 83 - protein, 166 aa, Artificial
  I-CreI_CFS1
MANTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLTKSKISGSAD

SEQ ID NO: 84 - protein, 141 aa, Artificial
  ColE7
MASGSKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGKGSSAD

SEQ ID NO: 85 - protein, 311 aa, Artificial
  hColE7Cre_D0101
MASGSKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGKGSDITKSKISEKMKGQGPSGNTKYNKEFLLYLAGFVDGDGSIIAQIKPNQSYKFKHQLSLTFQVTQKTQRRWFLDKLVDEIGVGYVRDRGSVSDYILSEIAPLHNFLTQLQPFLKLKQKQANLVLKIIEQLPSAKESPDKFLEVCTWVDQIAALNDSKTRKTTSETVRAVLDSAD

SEQ ID NO: 86 - protein, 314 aa, Artificial
  hColE7Cre_D0102
MASGSKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGKGSDITKSKISEKMKGLGPDGRKANTKYNKEFLLYLAGFVDGDGSIIAQIKPNQSYKFKHQLSLTFQVTQKTQRRWFLDKLVDEIGVGYVRDRGSVSDYILSEIAPLHNFLTQLQPFLKLKQKQANLVLKIIEQLPSAKESPDKFLEVCTWVDQIAALNDSKTRKTTSETVRAVLDSAD

SEQ ID NO: 87 - protein, 300 aa, Artificial
  hCreColE7_D0101
MANTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLTKSKISGSKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGKGSSAD

SEQ ID NO: 88 - protein, 177 aa, Artificial
  I-CreI_NFS1_N20
MAGSDITKSKISEKMKGQGPSGNTKYNKEFLLYLAGFVDGNGSIIAQIKPNQSYKFKHQLSLTFQVTQKTQRRWFLDKLVDEIGVGYVRDRGSVSDYILSEIAPLHNFLTQLQPFLKLKQKQANLVLKIIEQLPSAKESPDKFLEVCTWVDQIAALNDSKTRKTTSETVRAVLDSAD

SEQ ID NO: 89 - protein, 180 aa, Artificial
  I-CreI_NFS2_N20
MAGSDITKSKISEKMKGLGPDGRKANTKYNKEFLLYLAGFVDGNGSIIAQIKPNQSYKFKHQLSLTFQVTQKTQRRWFLDKLVDEIGVGYVRDRGSVSDYILSEIAPLHNFLTQLQPFLKLKQKQANLVLKIIEQLPSAKESPDKFLEVCTWVDQIAALNDSKTRKTTSETVRAVLDSAD

SEQ ID NO: 90 - protein, 166 aa, Artificial
  I-CreI_CFS1_N20
MANTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLTKSKISGSAD

SEQ ID NO: 91 - protein, 311 aa, Artificial
  hColE7Cre_D0101_N20
MASGSKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGKGSDITKSKISEKMKGQGPSGNTKYNKEFLLYLAGFVDGNGSIIAQIKPNQSYKFKHQLSLTFQVTQKTQRRWFLDKLVDEIGVGYVRDRGSVSDYILSEIAPLHNFLTQLQPFLKLKQKQANLVLKIIEQLPSAKESPDKFLEVCTWVDQIAALNDSKTRKTTSETVRAVLDSAD

SEQ ID NO: 92 - protein, 314 aa, Artificial
  hColE7Cre_D0102_N20
MASGSKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGKGSDITKSKISEKMKGLGPDGRKANTKYNKEFLLYLAGFVDGNGSIIAQIKPNQSYKFKHQLSLTFQVTQKTQRRWFLDKLVDEIGVGYVRDRGSVSDYILSEIAPLHNFLTQLQPFLKLKQKQANLVLKIIEQLPSAKESPDKFLEVCTWVDQIAALNDSKTRKTTSETVRAVLDSAD

SEQ ID NO: 93 - protein, 300 aa, Artificial
  hCreColE7_D0101_N20
MANTKYNKEFLLYLAGFVDGNGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLTKSKISGSKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGKGSSAD

SEQ ID NO: 94 - protein, 32 aa, Artificial
  RM2 peptidic linker
AAGGSALTAGALSLTAGALSLTAGALSGGGGS

SEQ ID NO: 95 - protein, 27 aa, Artificial
  BQY peptidic linker
AAGASSVSASGHIAPLSLPSSPPSVGS

SEQ ID NO: 96 - protein, 919 aa, Methylophilus methylotrophus
  MmeI ACC85607.1
MALSWNEIRRKAIEFSKRWEDASDENSQAKPFLIDFFEVFGITNKRVATFEHAVKKFAKAHKEQSRGFVDLFWPGILLIEMKSRGKDLDKAYDQALDYFSGIAERDLPRYVLVCDFQRFRLTDLITKESVEFLLKDLYQNVRSFGFIAGYQTQVIKPQDPINIKAAERMGKLHDTLKLVGYEGHALELYLVRLLFCLFAEDTTIFEKSLFQEYIETKTLEDGSDLAHHINTLFYVLNTPEQKRLKNLDEHLAAFPYINGKLFEEPLPPAQFDKAMREALLDLCSLDWSRISPAIFGSLFQSIMDAKKRRNLGAHYTSEANILKLIKPLFLDELWVEFEKVKNNKNKLLAFHKKLRGLTFFDPACGCGNFLVITYRELRLLEIEVLRGLHRGGQQVLDIEHLIQINVDQFFGIEIEEFPAQIAQVALWLTDHQMNMKISDEFGNYFARIPLKSTPHILNANALQIDWNDVLEAKKCCFILGNPPFVGKSKQTPGQKADLLSVFGNLKSASDLDLVAAWYPKAAHYIQTNANIRCAFVSTNSITQGEQVSLLWPLLLSLGIKINFAHRTFSWTNEASGVAAVHCVIIGFGLKDSDEKIIYEYESINGEPLAIKAKNINPYLRDGVDVIACKRQQPISKLPSMRYGNKPTDDGNFLFTDEEKNQFITNEPSSEKYFRRFVGGDEFINNTSRWCLWLDGADISEIRAMPLVLARIKKVQEFRLKSSAKPTRQSASTPMKFFYISQPDTDYLLIPETSSENRQFIPIGFVDRNVISSNATYHIPSAEPLIFGLLSSTMHNCWMRNVGGRLESRYRYSASLVYNTFPWIQPNEKQSKAIEEAAFAILKARSNYPNESLAGLYDPKTMPSELLKAHQKLDKAVDSVYGFKGPNTEIARIAFLFETYQKMTSLLPPEKEIKKSKGKN

SEQ ID NO: 97 - protein, 576 aa, Artificial
  Colicin-E7 (CEA7_ECOLX) Q47112.2
MSGGDGRGHNSGAHNTGGNINGGPTGLGGNGGASDGSGWSSENNPWGGGSGSGVHWGGGSGHGNGGGNSNSGGGSNSSVAAPMAFGFPALAAPGAGTLGISVSGEALSAAIADIFAALKGPFKFSAWGIALYGILPSEIAKDDPNMMSKIVTSLPAETVTNVQVSTLPLDQATVSVTKRVTDVVKDTRQHIAVVAGVPMSVPVVNAKPTRTPGVFHASFPGVPSLTVSTVKGLPVSTTLPRGITEDKGRTAVPAGFTFGGGSHEAVIRFPKESGQKPVYVSVTDVLTPAQVKQRQDEEKRLQQEWNDAHPVEVAERNYEQARAELNQANKDVARNQERQAKAVQVYNSRKSELDAANKTLADAKAEIKQFERFAREPMAAGHRMWQMAGLKAQRAQTDVNNKKAAFDAAAKEKSDADVALSSALERRKQKENKEKDAKAKLDKESKRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIHRGK

SEQ ID NO: 98 - protein, 274 aa, Streptococcus pneumoniae [sequence omitted]

SEQ ID NO: 99 - protein, 235 aa, Escherichia coli
  Endo I (END1_ECOLI) P25736.1
MYRYLSIAAVVLSAAFSGPALAEGINSFSQAKAAAVKVHADAPGTFYCGCKINWQGKKGVVDLQSCGYQVRKNENRASRVEWEHVVPAWQFGHQRQCWQDGGRKNCAKDPVYRKMESDMHNLQPSVGEVNGDRGNFMYSQWNGGEGQYGQCAMKVDFKEKAAEPPARARGAIARTYFYMRDQYNLTLSRQQTQLFNAWNKMYPVTDWECERDERIAKVQGNHNPYVQRACQARKS

SEQ ID NO: 100 - protein, 297 aa, Homo sapiens
  Human Endo G (NUCG_HUMAN) Q14249.4
MRALRAGLTLASGAGLGAVVEGWRRRREDARAAPGLLGRLPVLPVAAAAELPPVPGGPRGPGELAKYGLPGLAQLKSRESYVLCYDPRTRGALWVVEQLRPERLRGDGDRRECDFREDDSVHAYHRATNADYRGSGFDRGHLAAAANHRWSQKAMDDTFYLSNVAPQVPHLNQNAWNNLEKYSRSLTRSYQNVYVCTGPLFLPRTEADGKSYVKYQVIGKNHVAVPTHFFKVLILEAAGGQIELRTYVMPNAPVDEAIPLERFLVPIESIERASGLLFVPNILARAGSLKAITAGSK

SEQ ID NO: 101 - protein, 299 aa, Bos taurus
  Bovine Endo G (NUCG_BOVIN) P38447.1
MQLLRAGLTLALGAGLGAAAESWWRQRADARATPGLLSRLPVLPVAAAAGLPAVPGAPAGGGPGELAKYGLPGVAQLKSRASYVLCYDPRTRGALWVVEQLRPEGLRGDGNRSSCDFHEDDSVHAYHRATNADYRGSGFDRGHLAAAANHRWSQKAMDDTFYLSNVAPQVPHLNQNAWNNLEKYSRSLTRTYQNVYVCTGPLFLPRTEADGKSYVKYQVIGKNHVAVPTHFFKVLILEAAGGQIELRSYVMPNAPVDEAIPLEHFLVPIESIERASGLLFVPNILARAGSLKAITAGSK

SEQ ID NO: 102 - protein, 247 aa, Haemophilus influenzae
  R.HinP1I AAW33811.1
MNLVELGSKTAKDGFKNEKDIADRFENWKENSEAQDWLVTMGHNLDEIKSVKAVVLSGYKSDINVQVLVFYKDALDIHNIQVKLVSNKRGFNQIDKHWLAHYQEMWKFDDNLLRILRHFTGELPPYHSNTKDKRRMFMTEFSQEEQNIVLNWLEKNRVLVLTDILRGRGDFAAEWVLVAQKVSNNARWILRNINEVLQHYGSGDISLSPRGSINFGRVTIQRKGGDNGRETANMLQFKIDPTELFDI

SEQ ID NO: 103 - protein, 188 aa, Bacillus phage Bastille
  I-BasI AAO93095.1
MFQEEWKDVTGFEDYYEVSNKGRVASKRTGVIMAQYKINSGYLCIKFTVNKKRTSHLVHRLVAREFCEGYSPELDVNHKDTDRMNNNYDNLEWLTRADNLKDVRERGKLNTHTAREALAKVSKKAVDVYTKDGSEYIATYPSATEAAEALGVQGAKISTVCHGKRQHTGGYHFKFNSSVDPNRSVSKK

SEQ ID NO: 104 - protein, 266 aa, Bacillus mojavensis
  I-BmoI AAK09365.1
MKSGVYKITNKNTGKFYIGSSEDCESRLKVHFRNLKNNRHINRYLNNSFNKHGEQVFIGEVIHILPIEEAIAKEQWYIDNFYEEMYNISKSAYHGGDLTSYHPDKRNIILKRADSLKKVYLKMTSEEKAKRWQCVQGENNPMFGRKHTETTKLKISNHNKLYYSTHKNPFKGKKHSEESKTKLSEYASQRVGEKNPFYGKTHSDEFKTYMSKKFKGRKPKNSRPVIIDGTEYESATEASRQLNVVPATILHRIKSKNEKYSGYFYK

SEQ ID NO: 105 - protein, 174 aa, Artificial; identical to SEQ ID NO: 55
  I-HmuI P34081.1

SEQ ID NO: 106 - protein, 245 aa, Artificial; identical to SEQ ID NO: 60
  I-TevI P13299.2

SEQ ID NO: 107 - protein, 258 aa, Artificial
  I-TevII P07072.2
MKWKLRKSLKIANSVAFTYMVRFPDKSFYIGFKKFKTIYGKDTNWKEYNSSSKLVKEKLKDYKAKWIILQVFDSYESALKHEEMLIRKYFNNEFILNKSIGGYKFNKYPDSEEHKQKLSNAHKGKILSLKHKDKIREKLIEHYKNNSRSEAHVKNNIGSRTAKKTVSIALKSGNKFRSFKSAAKFLKCSEEQVSNHPNVIDIKITIHPVPEYVKINDNIYKSFVDAAKDLKLHPSRIKDLCLDDNYPNYIVSYKRVEK

SEQ ID NO: 108 - protein, 269 aa, Artificial
  I-TevIII Q38419.1
MNYRKIWIDANGPIPKDSDGRTDEIHHKDGNRENNDLDNLMCLSIQEHYDIHLAQKDYQACHAIKLRMKYSPEEISELASKAAKSREIQIFNIPEVRAKNIASIKSKIENGTFHLLDGEIQRKSNLNRVALGIHNFQQAEHIAKVKERNIAAIKEGTHVFCGGKMQSETQSKRVNDGSHHFLSEDHKKRTSAKTLEMVKNGTHPAQKEITCDFCGHIGKGPGFYLKHNDRCKLNPNRIQLNCPYCDKKDLSPSTYKRWHGDNCKARFND

SEQ ID NO: 109 - protein, 243 aa, Staphylococcus phage Twort
  I-TwoI AAM00817.1
MEELWKEIPGFNSYMISNKGQVYSRKRNKILALRTDKNGYKRISIFNNEGKRILLGVHKLVLLGFKGINTEKPIPHHKNNIKDDNRLENLEWVTVSENTKHAYDIGALKSPRRVTCTLYYKGEPLSCYDSLFDLAKALKVSRSVIESPRNGLVLSTFEVKREPTIQGLPLNKEIFEHSLIKGLGNPPLKVYNEDETYYFLTLMDISKYFNESYSKVQRGYYKGKWKSYIIEHIDFYEYYKQTH

SEQ ID NO: 110 - protein, 262 aa, Artificial
  R.MspI P11405.1
MRTELLSKLYDDFGIDQLPHTQHGVTSDRLGKLYEKYILDIFKDIESLKKYNTNAFPQEKDISSKLLKALNLDLDNIIDVSSSDTDLGRTIAGGSPKTDATIRFTFHNQSSRLVPLNIKHSSKKKVSIAEYDVETICTGVGISDGELKELIRKHQNDQSAKLFTPVQKQRLTELLEPYRERFIRWCVTLRAEKSEGNILHPDLLIRFQVIDREYVDVTIKNIDDYVSDRIAEGSKARKPGFGTGLNWTYASGSKAKKMQFKG

SEQ ID NO: 111 - protein, 246 aa, Kocuria varians
  R.MvaI
MSEYLNLLKEAIQNVVDGGWHETKRKGNTGIGKTFEDLLEKEEDNLDAPDFHDIEIKTHETAAKSLLTLFTKSPTNPRGANTMLRNRYGKKDEYGNNILHQTVSGNRKTNSNSYNYDFKIDIDWESQVVRLEVFDKQDIMIDNSVYWSFDSLQNQLDKKLKYIAVISAESKIENEKKYYKYNSANLFTDLTVQSLCRGIENGDIKVDIRIGAYHSGKKKGKTHDHGTAFRINMEKLLEYGEVKVIV

SEQ ID NO: 112 - protein, 274 aa, Nostoc sp. PCC 7120
  NucA CAA45962.1
MGICGKLGVAALVALIVGCSPVQSQVPPLTELSPSISVHLLLGNPSGATPTKLTPDNYLMVKNQYALSYNNSKGTANWVAWQLNSSWLGNAERQDNFRPDKTLPAGWVRVTPSMYSGSGYDRGHIAPSADRTKTTEDNAATFLMTNMMPQTPDNNRNTWGNLEDYCRELVSQGKELYIVAGPNGSLGKPLKGKVTVPKSTWKIVVVLDSPGSGLEGITANTRVIAVNIPNDPELNNDWRAYKVSVDELESLTGYDFLSNVSPNIQTSIESKVDN

SEQ ID NO: 113 - protein, 232 aa, Artificial
  NucM P37994.2
MLRNLVIFAVLGAGLTTLAAAGQDINNFTQAKAAAAKIHQDAPGTFYCGCKINWQGKKGTPDLASCGYQVRKDANRASRIEWEHVVPAWQFGHQRQCWQDGGRKNCTKDDVYRQIETDLHNLQPAIGEVNGDRGNFMYSQWNGGERQYGQCEMKIDFKSQLAEPPERARGAIARTYFYMRDRYNLNLSRQQTQLFDAWNKQYPATTWECTREKRIAAVQGNHNPYVQQACQP

SEQ ID NO: 114 - protein, 231 aa, Vibrio vulnificus
  Vvn AAF19759.1
MKRLFIFIASFTAFAIQAAPPSSFSAAKQQAVKIYQDHPISFYCGCDIEWQGKKGIPNLETCGYQVRKQQTRASRIEWEHVVPAWQFGHHRQCWQKGGRKNCSKNDQQFRLMEADLHNLTPAIGEVNGDRSNFNFSQWNGVDGVSYGRCEMQVNFKQRKVMPQTELRGSIARTYLYMSQEYGFQLSKQQQQLMQAWNKSYPVDEWECTRDDRIAKIQGNHNPFVQQSCQTQ

SEQ ID NO: 115 - protein, 222 aa, Artificial
  Vvn_CLS
MASGAPPSSFSAAKQQAVKIYQDHPISFYCGCDIEWQGKKGIPNLETCGYQVRKQQTRASRIEWEHVVPAWQFGHHRQCWQKGGRKNCSKNDQQFRLMEADLHNLTPAIGEVNGDRSNFNFSQWNGVDGVSYGRCEMQVNFKQRKVMPPDRARGSIARTYLYMSQEYGFQLSKQQQQLMQAWNKSYPVDEWECTRDDRIAKIQGNHNPFVQQSCQTQGSSAD

SEQ ID NO: 116 - protein, 231 aa, Artificial
  Staphylococcal nuclease (NUC_STAAU) P00644.1
MLVMTEYLLSAGICMAIVSILLIGMAISNVSKGQYAKRFFFFATSCLVLTLVVVSSLSSSANASQTDNGVNRSGSEDPTVYSATSTKKLHKEPATLIKAIDGDTVKLMYKGQPMTFRLLLVDTPETKHPKKGVEKYGPEASAFTKKMVENAKKIEVEFDKGQRTDKYGRGLAYIYADGKMVNEALVRQGLAKVAYVYKPNNTHEQHLRKSEAQAKKEKLNIWSEDNADSGQ

SEQ ID NO: 117 - protein, 169 aa, Artificial
  Staphylococcal nuclease (NUC_STAHY) P43270.1
MKKITTGLIIVVAAIIVLSIQFMTESGPFKSAGLSNANEQTYKVIRVIDGDTIIVDKDGKQQNLRMIGVDTPETVKPNTPVQPYGKEASDFTKRHLTNQKVRLEYDKQEKDRYGRTLAYVWLGKEMFNEKLAKEGLARAKFYRPNYKYQERIEQAQKQAQKLKKNIWSN

SEQ ID NO: 118 - protein, 174 aa, Artificial
  Micrococcal nuclease (NUC_SHIFL)P29769.1
MKSALAALRAVAAAVVLIVSVPAWADFRGEVVRILDGDTIDVLVNRQTIRVRLADIDAPESGQAFGSRARQRLADLTFRQEVQVTEKEVDRYGRTLGVVYAPLQYPGGQTQLTNINAIMVQEGMAWAYRYYGKPTDAQMYEYEKEARRQRLGLWSDPNAQEPWKWRRASKNATN

SEQ ID NO: 119 - protein, 211 aa, Artificial
  Endonuclease yncB P94492.1
MKKILISMIAIVLSITLAACGSNHAAKNHSDSNGTEQVSQDTHSNEYNQTEQKAGTPHSKNQKKLVNVTLDRAIDGDTIKVIYNGKKDTVRYLLVDTPETKKPNSCVQPYGEDASKRNKELVNSGKLQLEFDKGDRRDKYGRLLAYVYVDGKSVQETLLKEGLARVAYVYEPNTKYIDQFRLDEQEAKSDKLSIWSKSGYVTNRGFNGCVK

SEQ ID NO: 120 - protein, 149 aa, Artificial
  Endodeoxyribonuclease I (ENRN_BPT7)P00641.1
MAGYGAKGIRKVGAFRSGLEDKVSKQLESKGIKFEYEEWKVPYVIPASNHTYTPDFLLPNGIFVETKGLWESDDRKKHLLIREQHPELDIRIVFSSSRTKLYKGSPTSYGEFCEKHGIKFADKLIPAEWIKEPKKEVPFDRLKRKGGKK

SEQ ID NO: 121 - protein, 671 aa, Homo sapiens
  Metnase Q53H47.1
MAEFKEKPEAPTEQLDVACGQENLPVGAWPPGAAPAPFQYTPDHVVGPGADIDPTQITFPGCICVKTPCLPGTCSCLRHGENYDDNSCLRDIGSGGKYAEPVFECNVLCRCSDHCRNRVVQKGLQFHFQVFKTHKKGWGLRTLEFIPKGRFVCEYAGEVLGFSEVQRRIHLQTKSDSNYIIAIREHVYNGQVMETFVDPTYIGNIGRFLNHSCEPNLLMIPVRIDSMVPKLALFAAKDIVPEEELSYDYSGRYLNLTVSEDKERLDHGKLRKPCYCGAKSCTAFLPFDSSLYCPVEKSNISCGNEKEPSMCGSAPSVFPSCKRLTLETMKMMLDKKQIRAIFLFEFKMGRKAAETTRNINNAFGPGTANERTVQWWFKKFCKGDESLEDEERSGRPSEVDNDQLRAIIEADPLTTTREVAEELNVNHSTVVRHLKQIGKVKKLDKWVPHELTENQKNRRFEVSSSLILRNHNEPFLDRIVTCDEKWILYDNRRRSAQWLDQEEAPKHFPKPILHPKKVMVTIWWSAAGLIHYSFLNPGETITSEKYAQEIDEMNQKLQRLQLALVNRKGPILLHDNARPHVAQPTLQKLNELGYEVLPHPPYSPDLLPTNYHVFKHLNNFLQGKRFHNQQDAENAFQEFVESQSTDFYATGINQLISRWQKCVDCNGSYFD

SEQ ID NO: 122 - protein, 488 aa, Geobacillus stearothermophilus
  Nb.BsrDI ABD15132.1
MTEYDLHLYADSFHEGHWCCENLAKIAQSDGGKHQIDYLQGFIPRHSLIFSDLIINITVFGSYKSWKHLPKQIKDLLFWGKPDFIAYDPKNDKILFAVEETGAVPTGNQALQRCERIYGSARKQIPFWYLLSEFGQHKDGGTRRDSIWPTIMGLKLTQLVKTPSIILHYSDINNPEDYNSGNGLKFLFKSLLQIIINYCTLKNPLKGMLELLSIQYENMLEFIKSQWKEQIDFLPGEEILNTKTKELARMYASLAIGQTVKIPEELFNWPRTDKVNFKSPQGLIKYDELCYQLEKAVGSKKAYCLSNNAGAKPQKLESLKEWINSQKKLFDKAPKLTPPAEFNMKLDAFPVTSNNNYYVTTSKNILYLFDYWKDLRIAIETAFPRLKGKLPTDIDEKPALIYICNSVKPGRLFGDPFTGQLSAFSTIFGKKNIDMPRIVVAYYPHQIYSQALPKNNKSNKGITLKKELTDFLIFHGGVVVKLNEGKAY

SEQ ID NO: 123 - protein, 217 aa, Geobacillus stearothermophilus
  BsrDI A ABD15133.1
MTDYRYSFELSEEIARWAFEIKTKNTDWFVAFSNPTAGPWKRVMAIDKASNREGEVHRFGREDERPDIILVNDNISLILILEAKEKLNQLISKSQVDKSVDVFLTLSSILKEKSDNNYWGDRTKYINVLGILWGSEQETSQKDIDNAFRVYRDSLVKNLKEINPTPTNICTDILVGVESIKNKKEEISIKIHVSNIYAEIYPKFTGKHLLEKLAVLN

SEQ ID NO: 124 - protein, 604 aa, Bacillus sp. D6
  Nt.BspD6I ABN42182.1 (R.BspD6I large subunit)
MAKKVNWYVSCSPRSPEKIQPELKVLANFEGSYWKGVKGYKAQEAFAKELAALPQFLGTTYKKEAAFSTRDRVAPMKTYGFVFVDEEGYLRITEAGKMLANNRRPKDVFLKQLVKWQYPSFQHKGKEYPEEEWSINPLVFVLSLLKKVGGLSKLDIAMFCLTATNNNQVDEIAEEIMQFRNEREKIKGQNKKLEFTENYFFKRFEKIYGNVGKIREGKSDSSHKSKIETKMRNARDVADATTRYFRYTGLFVARGNQLVLNPEKSDLIDEIISSSKVVKNYTRVEEFHEYYGNPSLPQFSFETKEQLLDLAHRIRDENTRLAEQLVEHFPNVKVEIQVLEDIYNSLNKKVDVETLKDVIYHAKELQLELKKKKLQADFNDPRQLEEVIDLLEVYHEKKNVIEEKIKARFIANKNTVFEWLTWNGFIILGNALEYKNNFVIDEELQPVTHAAGNQPDMEIIYEDFIVLGEVTTSKGATQFKMESEPVTRHYLNKKKELEKQGVEKELYCLFIAPEINKNTFEEFMKYNIVQNTRIIPLSLKQFNMLLMVQKKLIEKGRRLSSYDIKNLMVSLYRTTIECERKYTQIKAGLEETLNNWVVDKEVRF

SEQ ID NO: 125 - protein, 186 aa, Bacillus sp. D6
  ss.BspD6I (R.BspD6I small subunit)
MQDILDFYEEVEKTINPPNYFEWNTYRVFKKLGSYKNLVPNFKLDDSGHPIGNAIPGVEDILVEYEHFSILIECSLTIGEKQLDYEGDSVVRHLQEYKKKGIEAYTLFLGKSIDLSFARHIGFNKESEPVIPLTVDQFKKLVTQLKGDGEHFNPNKLKEILIKLLRSDLGYDQAEEWLTFIEYNLK

SEQ ID NO: 126 - protein, 555 aa, Paucimonas lemoignei
  R.PleI AAK27215.1
MAKPIDSKVLFITTSPRTPEKMVPEIELLDKNFNGDVWNKDTQTAFMKILKEESFFDGEGKNDPAFSARDRINRAPKSLGFVILTPKLSLTDAGVELIKAKRKDDIFLRQMLKFQLPSPYHKLSDKAALFYVKPYLEIFRLVRHFGSLTFDELMIFGLQIIDFRIFNQIVDKIEDFRVGKIENKGRYKTYKKERFEEELGKIYKDELFGLTEASAKTLITKKGNNMRDYADACVRYLRATGMVNVSYQGKSLSIVQEKKEEVDFFLKNTEREPCFINDEASYVSYLGNPNYPKLFVDDVDRIKKKLRFDFKKTNKVNALTLPELKEELENEILSRKENILKSQISDIKNFKLYEDIQEVFEKIENDRTLSDAPLMLEWNTWRAMTMLDGGEIKANLKFDDFGSPMSTAIGNMPDIVCEYDDFQLSVEVTMASGQKQYEMEGEPVSRHLGKLKKSSEKPVYCLFIAPKINPSSVAHFFMSHKVDIEYYGGKSLIIPLELSVFRKMIEDTFKASYIPKSDNVHKLFKNFASIADEAGNEKVWYEGVKRTAMNWLSLS

SEQ ID NO: 127 - protein, 556 aa, Micrococcus lylae
  MlyI AAK39546.1
MASLSKTKHLFGFTSPRTIEKIIPELDILSQQFSGKVWGENQINFFDAIFNSDFYEGTTYPQDPALAARDRITRAPKALGFIQLKPVIQLTKAGNQLVNQKRLPELFTKQLLKFQLPSPYHTQSPTVNFNVRPYLELLRLINELGSISKTEIALFFLQLVNYNKFDEIKNKILKFRETRKNNRSVSWKTYVSQEFEKQISIIFADEVTAKNFRTRESSDESFKKFVKTKEGNMKDYADAFFRYIRGTQLVTIDKNLHLKISSLKQDSVDFLLKNTDRNALNLSLMEYENYLFDPDQLIVLEDNSGLINSKIKQLDDSINVESLKIDDAKDLLNDLEIQRKAKTIEDTVNHLKLRSDIEDILDVFAKIKKRDVPDVPLFLEWNIWRAFAALNHTQAIEGNFIVDLDGMPLNTAPGKKPDIEINYGSFSCIVEVTMSSGETQFNMEGSSVPRHYGDLVRKVDHDAYCIFIAPKVAPGTKAHFFNLNRLSTKHYGGKTKIIPMSLDDFICFLQVGITHNFQDINKLKNWLDNLINFNLESEDEEIWFEEIISKISTWAI

SEQ ID NO: 128 - protein, 543 aa, Geobacillus sp. Y412MC52
  AlwI YP_004134094.1
MNKKNTRKVWFITRPERDPRFHQEALLALQKATDDFRLKWAGNREVHKRYEEELANMGIKRNNVSHDGSGGRTWMAMLKTFSYCYVDDDGYIRLTKVGEKLIQGEKVYENTRKQVLTLQYPNAYFLEPGFRPKFDEGFRIRPVLFLIKLANDERLDFYVTKEEITYFAMTAQKDSQLDEIVHKILAFRKAGPREREEMKQDIAAKFDHRERSDKGARDFYEAHSDVAHTFMLISDYTGLVEYIRGKALKGDSSKINEIKQEIAEIEKRYPFNTRYMISLERMAENSGLDVDSYKASRYGNIKPAANSSKLRAKAERILAQFPSIESMSKEEIAGALQKYLSPRDIEKVIHEIVENKDDFEGINSDFVETYLNEKDNLAFEDKTGQIFSALGFDVAMRPKAKNGERTEIEIIARYGGSKFGIIDAKNYAGKFPLSSSLVSHMASEYIPNYTGYEGKELTFFGYVTANDFSGERNLEKISDKAKRITGNPISGFLVTARTLLGFLDYCIENDVPLEDRAELFVKAVKNKGYKSLEALLRELKETI

SEQ ID NO: 129 - protein, 685 aa, Kocuria varians
  Mva1269I AAY97906.1
MYLNTAVFNIYGDNIVECSRAFHYILEGFKLANISITQEYDLQNITTPKFCIYTDKFRYIFIFIPGTSASRWNKDIYKELVLNNGGPLKEGADAIITRIFSEDSELVLASMEFSAALPAGNNTWQRSGRAYSLTAANIPYFYIVQLGGKEIKKGKDGKSDKFATRLPNPALSLSFTLNTIKKPAPSLIVYDQAPEADSAISDLYSNCYGIDDFSLYLFKLITEENNLHELKNIYNKNVEFLQLRSVDEKGKNFSGKDYKYIFEHKDPYKGLTEVVKERKIPWKKKTATKTFENFPLRNQAPIFRLIDFLSTKSYGIVSKDSLPLTFIPSEHRVEVANYICNQLYIDKVSDEFVKWIYKKEDLAICIINGFKPGGDDSRPDRGLPPFTKMLTNLDILTLMFGPAPPTQWDYLDSDPEKLNKTNGLWQSIFAFSDAILVDSSTRDNNKFVYNAYLKEHWVVQREKKESNTPISYFPKSVGEHDVDTSLHILFTYIGKHFESACNPPGGDWSGVSLLKNNIEYRWTSMYRVSQDGTKRPDHIYQLVYNSTDTLLLIESKGIKNDLLKSKEANVGIGMINYLKNLMARDYTAVKKDGEWKNIHGQMTLDKFLTFSAVAYLFTTDFDNEYTSAAELLVHSNTQLAFALEIKEKNSVMHIFTANTVAYNFAEYLLETMRNSHLPLKIYKPI

SEQ ID NO: 130 - protein, 599 aa, Geobacillus stearothermophilus
  BsrI ADR72996.1
MRNIRIYSEVKEQGIFFKEVIQSVLEKANVEVVLVNSAMLDYSDVSVISLIRNQKKFDLLVSEVRDKREIPIVMVEFSTAVTTDDHELQRADAMFWAYKYKIPYLKISPMEKKSQTADDKFGGGRLLSVNDQIIHMYRTDGVMYHIEWESMDNSAYVKNAELYPSCPDCAPELASLFRCLLETIEKCENIEDYYRILLDKLGKQKVAVKWGNFREEKTLEQWKHEKFDLLERFSKSSSRMEYDKDKKELKIKVNRYGHAMDPERGILAFWKLVLGDEWKIVAEFQLQRKTLKGRQSYQSLFDEVSQEEKLMNIASEIIKNGNVISPDKAIEIHKLATSSTMISTIDLGTPERKYITDDSLKGYLQHGLITNIYKNLLYYVDEIRFTDLQRKTIASLTWNKEIVNDYYKSLMDQLLDKNLRVLPLTSIKNISEDLITWSSKEILINLGYKILAASYPEAQGDRCILVGPTGKKTERKFIDLIAISPKSKGVILLECKDKLSKSKDDCEKMNDLLNHNYDKVTKLINVLNINNYNYNNIIYTGVAGLIGRKNVDNLPVDFVIKFKYDAKNLKLNWEINSDILGKHSGSFSMEDVAVVRKRS

SEQ ID NO: 131 - protein, 676 aa, Geobacillus stearothermophilus
  BsmI AAL86024.1
MNVFRIHGDNIIECERVIDLILSKINPQKVKRGFISLSCPFIEIIFKEGHDYFHWRFDMFPGFNKNTNDRWNSNILDLLSQKGSFLYETPDVIITSLNNGKEEILMAIEFCSALQAGNQAWQRSGRAYSVGRTGYPYIYIVDFVKYELNNSDRSRKNLRFPNPAIPYSYISHSKNTGNFIVQAYFRGEEYQPKYDKKLKFFDETIFAEDDIADYIIAKLQHRDTSNIEQLLINKNLKMVEFLSKNTKNDNNFTYSEWESIYNGTYRITNLPSLGRFKFRKKIAEKSLSGKVKEFNNIVQRYSVGLASSDLPFGVIRKESRNDFINDVCKLYNINDMKIIKELKEDADLIVCMLKGFKPRGDDNRPDRGALPLVAMLAGENAQIFTFIYGPLIKGAINLIDQDINKLAKRNGLWKSFVSLSDFIVLDCPIIGESYNEFRLIINKNNKESILRKTSKQQNILVDPTPNHYQENDVDTVIYSIFKYIVPNCFSGMCNPPGGDWSGLSIIRNGHEFRWLSLPRVSENGKRPDHVIQILDLFEKPLLLSIESKEKPNDLEPKIGVQLIKYIEYLFDFTPSVQRKIAGGNWEFGNKSLVPNDFILLSAGAFIDYDNLTENDYEKIFEVTGCDLLIAIKNQNNPQKWVIKFKPKNTIAEKLVNYIKLNFKSNIFDTGFFHIEG

SEQ ID NO: 132 - protein, 465 aa, Artificial
  Nb.BtsCI ADI24225.1
MKRILYLLTEERPKINIIHQIINLEYKATLHFGAKIVPVMNEENKFTFIYHVKGIEVEGFDAVLIKIVSGHSSFVDYLVFDSNDLKPEKNTITLFDLDQYELDLSYYFGKGWIVRIPSPSDLPKYVVEETKTDDHESRNTNAYQRSSKFVFCELYYGKEVKKYMLYDISDGRTLSGTDTHNFGMRMLVTNNVNLVGVPNMYLPFTDIKEFINEKNRIADNGPSHNVPIRLKLDKEKNVIYISAKLDKGNGKNKNKISNDPNIGAVAIISATLRNLNWKGDIEIINHNLLPSSISSRSNGNKLLYIMKKLGVRFNNINVNWNNIKNNINYFFYNITSEKIVSIYYHLYVEDKLSNARVIFDNHAGCGKSYFRTLNNKIIPVGKEIPLPALVIFDSDQNIVKVIAAAKAENVYNGVEQLSTFDKFIESYINKYYPGAAVECSVITWGKSSNPYVSFYLDKDGSAVFL

SEQ ID NO: 133 - protein, 465 aa, Artificial
  Nt.BtsCI ADI24224.1
MKRILYLLTEERPKINIIHQIINLEYKATLHFGAKIVPVMNEENKFTFIYHVKGIEVEGFDAVLIKIVSGHSSFVDYLVFDSNDLKPEKNTITLFDLDQYELDLSYYFGKGWIVRIPSPSDLPKYVVFETKTDDHESRNTNAYQRSSKFVFCELYYGKEVKKYMLYDISDGRTLSGTDTHNFGMRMLVTNNVNLVGVPNMYLPFTDIKEFINEKNRIADNGPSHNVPIRLKLDKEKNVIYISAKLDKGNGKNKNKISNDPNIGAVAIISATLRNLNWKGDIEIINHNLLPSSISSRSNGNKLLYIMKKLGVRFNNINVNWNNIKNNINYFFYNITSEKIVSIYYHLYVEDKLSNARVIFDNHAGCGKSYFRTLNNKIIPVGKEIPLPDLVIFDSDQNIVKVIEAEKAENVYNGVEQLSTFDKFIESYINKYYPGAAVECSVITWGKSSNPYVSFYLDKDGSAVFL

SEQ ID NO: 134 - protein, 164 aa, Geobacillus thermoglucosidasius
  R1.BtsI ABC75874.1
MKITEGIVHVAMRHFLKSNGWKLIAGQYPGGSDDELTALNIVDPVVARDNSPDPRRHSLGKIVPDLIAYKNDDLLVIEAKPKYSQDDRDKLLYLLSERKHDFYAALEKFATERNHPELLPVSKLNIIPGLAFSASENKFKKDPGFVYIRVSGIFEAFMEGYDWG

SEQ ID NO: 135 - protein, 328 aa, Geobacillus thermoglucosidasius
  R2.BtsI ABC75876.1
MQIEQLMKSLTIYFDDIQEGLWFKNLHPLLESASLEAITGSLKRNPNLADVLKYDRPDIILTLNQTPILVIERTIEVPSGHNVGQRYGRLAAASEAGVPLVYFGPYAARKHGGATEGPRYMNLRLFYALDVMQKVNGSAITTINWPVDQNFEILQDPSKDKRMKEYLEMFFDNLLKYGIAGINLAIRNSSFQAEQLAEREKFVETMITNPEQYDVPPDSVQILNAERFFNELGISENKRIICDEVVLYQVGMTYVRSDPYTGMALLYKYLYILGSERNRCLILKFPNITTDMWKKVAFGSRERKDVRIYRSVSDGILFADGYLSKEEL

SEQ ID NO: 136 - protein, 275 aa, Brevibacillus brevis
  BbvCI subunit 1 AAX14652.1
MINEDFFIYEQLSHKKNLEQKGKNAFDEETEELVRQAKSGYHAFIEGINYDEVTKLDLNSSVAALEDYISIAKEIEKKHKMFNWRSDYAGSIIPEFLYRIVHVATVKAGLKPIFSTRNTIIEISGAAHREGLQIRRKNEDFALGFHEVDVKIASESHRVISLAVACEVKTNIDKNKLNGLDFSAERMKRTYPGSAYFLITETLDFSPDENHSSGLIDEIYVLRKQVRTKNRVQKAPLCPSVFAELLEDILEISYRASNVKGHVYDRLEGGKLIRV

SEQ ID NO: 137 - protein, 285 aa, Brevibacillus brevis
  BbvCI subunit 2 AAX14653.1
MFNQFNPLVYTHGGKLERKSKKDKTASKVFEEFGVMEAYNCWKEASLCIQQRDKDSVLKLVAALNTYKDAVEPIFDSRLNSAQEVLQPSILEEFFEYLFSRIDSIVGVNIPIRHPAKGYLSLSFNPHNIETLIQSPEYTVRAKDHDFIIGGSAKLTIQGHGGEGETTNIVVPAVAIECKRYLERNMLDECAGTAERLKRATPYCLYFVVAEYLKLDDGAPELTEIDEIYILRHQRNSERNKPGFKPNPIDGELIWDLYQEVMNHLGKIWWDPNSALQRGKVFNRP

SEQ ID NO: 138 - protein, 294 aa, Bacillus pumilus
  Bpu10I alpha subunit CAA74998.1
MGVEQEWIKNITDMYQSPELIPSHASNLLHQLKREKRNEKLKKALEIITPNYISYISILLNNHNMTRKEIVILVDALNEYMNTLRHPSVKSVFSHQADFYSSVLPEFFNLLFRNLIKGLNEKIKVNSQKDIIIDCIFDPYNEGRVVFKKKRVDVAIILKNKFVFNNVEISDFAIPLVAIEIKTNLDKNMLSGIEQSVDSLKETFPLCLYYCITELADFAIEKQNYASTHIDEVFILRKQKRGPVRRGTPLEVVHADLILEVVEQVGEHLSKFKDPIKTLKARMTEGYLIKGKGK

SEQ ID NO: 139 - protein, 288 aa, Bacillus pumilus
  Bpu10I beta subunit CAA74999.1
MTQIDLSNTKHGSILFEKQKNVKEKYLQQAYKHYLYFRRSIDGLEITNDEAIFKLTQAANNYRDNVLYLFESRPNSGQEAFRYTILEEFFYHLFKDLVKKKFNQEPSSIVMGKANSYVSLSFSPESFLGLYENPIPYIHTKDQDFVLGCAVDLKISPKNELNKENETEIVVPVIAIECKTYIERNMLDSCAATASRLKAAMPYCLYIVASEYMKMDQAYPELTDIDEVFILCKASVGERTALKKKGLPPHKLDENLMVELFHMVERHLNRVWWSPNEALSRGRVIGRP

SEQ ID NO: 140 - protein, 358 aa, Bacillus megaterium
  BmrI ABM69266.1
MNYFSLHPNVYATGRPKGLINMLESVWISNQKPGDGTMYLISGFANYNGGIRFYETFTEHINHGGKVIAILGGSTSQRLSSKQVVAELVSRGVDVYIINRKRLLHAKLYGSSSNSGESLVVSSGNFTGPGMSQNVEASLLLDNNTTSSMGFSWNGMVNSMLDQKWQIHNLSNSNPTSPSWNLLYDERTTNLTLDDTQKVTLILTLGHADTARIQAAPKSKAGEGSQYFWLSKDSYDFFPPLTIRNKRGTKATYSCLINMNYLDIKYIDSECRVTFEAENNFDFRLGTGKLRYTNVAASDDIAAITRVGDSDYELRIIKKGSSNYDALDSAAVNFIGNRGKRYGYIPNDEFGRIIGAKF

SEQ ID NO: 141 - protein, 358 aa, Bacillus firmus
  BfiI CAC12783.1
MNFFSLHPNVYATGRPKGLIGMLENVWVSNHTPGEGTLYLISGFSNYNGGVRFYETFTEHINQGGRVIAILGGSTSQRLSSRQVVEELLNRGVEVHIINRKRILHAKLYGTSNNLGESLVVSSGNFTGPGMSQNIEASLLLDNNTTQSMGFSWNDMISEMLNQNWHIHNMTNATDASPGWNLLYDERTTNLTLDETERVTLIVTLGHADTARIQAAPGTTAGQGTQYFWLSKDSYDFFPPLTIRNRRGTKATYSSLINMNYIDINYTDTQCRVTFEAENNFDFRLGTGKLRYTGVAKSNDIAAITRVGDSDYELRIIKQGTPEHSQLDPYAVSFIGNRGKRFGYISNEEFGRIIGVTF

SEQ ID NO: 142 - protein, 846 aa, Homo sapiens
  hExoI (EXO1_HUMAN)  Q9UQ84.2
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 143 - protein, 702 aa, Saccharomyces cerevisiae
  Yeast ExoI (EXO1_YEAST) P39875.2
MGIQGLLPQLKPIQNPVSLRRYEGEVLAIDGYAWLHRAACSCAYELAMGKPTDKYLQFFIKRFSLLKTFKVEPYLVFDGDAIPVKKSTESKRRDKRKENKAIAERLWACGEKKNAMDYFQKCVDITPEMAKCIICYCKLNGIRYIVAPFEADSQMVYLEQKNIVQGIISEDSDLLVFGCRRLITKLNDYGECLEICRDNFIKLPKKFPLGSLTNEEIITMVCLSGCDYTNGIPKVGLITAMKLVRRFNTIERIILSIQREGKLMIPDTYINEYEAAVLAFQFQRVFCPIRKKIVSLNEIPLYLKDTESKRKRLYACIGFVIHRETQKKQIVHFDDDIDHHLHLKIAQGDLNPYDFHQPLANREHKLQLASKSNIEFGKTNTTNSEAKVKPIESFFQKMTKLDHNPKVANNIHSLRQAEDKLTMAIKRRKLSNANVVQETLKDTRSKFFNKPSMTVVENFKEKGDSIQDFKEDTNSQSLEEPVSESQLSTQIPSSFITTNLEDDDNLSEEVSEVVSDIEEDRKNSEGKTIGNEIYNTDDDGDGDTSEDYSETAESRVPTSSTTSFPGSSQRSISGCTKVLQKFRYSSSFSGVNANRQPLFPRHVNQKSRGMVYVNQNRDDDCDDNDGKNQITQRPSLRKSLIGARSQRIVIDMKSVDERKSFNSSPILHEESKKRDIETTKSSQARPAVRSISLLSQFVYKGK

SEQ ID NO: 144 - protein, 475 aa, Escherichia coli DH1
  E.coli ExoI BAJ43803.1
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIV

SEQ ID NO: 145 - protein, 279 aa, Homo sapiens
  TREX2_HUMAN Q9BQ50.1
MGRAGSPLPRSSWPRMDDCGSRSRCSPTLCSSLRTCYPRGNITMSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 146 - protein, 314 aa, Mus musculus
  TREX1_MOUSE Q91XB0.2
MGSQTLPHGHMQTLIFLDLEATGLPSSRPEVTELCLLAVHRRALENTSISQGHPPPVPRPPRVVDKLSLCIAPGKACSPGASEITGLSKAELEVQGRQRFDDNLAILLRAFLQRQPQPCCLVAHNGDRYDFPLLQTELARLSTPSPLDGTFCVDSIAALKALEQASSPSGNGSRKSYSLGSIYTRLYWQAPTDSHTAEGDVLTLLSICQWKPQALLQWVDEHARPFSTVKPMYGTPATTGTTNLRPHAATATTPLATANGSPSNGRSRRPKSPPPEKVPEAPSQEGLLAPLSLLTLLTLAIATLYGLFLASPGQ

SEQ ID NO: 147 - protein, 369 aa, Homo sapiens
  TREX1_HUMAN  Q9NSU2.1
MGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 148 - protein, 315 aa, Bos taurus
  TREX1_BOVIN  Q9BG99.1
MGSRALPPGPVQTLIFLDLEATGLPFSQPKITELCLLAVHRYALEGLSAPQGPSPTAPVPPRVLDKLSLCVAPGKVCSPAASEITGLSTAVLAAHGRRAFDADLVNLIRTFLQRQPQPWCLVAHNGDRYDFPLLRAELALLGLASALDDAFCVDSIAALKALEPTGSSSEHGPRKSYSLGSVYTRLYGQAPPDSHTAEGDVLALLSVCQWRPRALLRWVDAHAKPFSTVKPMYVITTSTGTNPRPSAVTATVPLARASDTGPNLRGDRSPKPAPSPKMCPGAPPGEGLLAPLGLLAFLTLAVAMLYGLSLAMPGQ

SEQ ID NO: 149 - protein, 316 aa, Rattus norvegicus
  Rat TREX1 AAH91242.1
MGSQALPHGHMQTLIFLDLEATGLPYSQPKITELCLLAVHRHALENSSMSEGQPPPVPKPPRVVDKLSLCIAPGKPCSSGASEITGLTTAGLEAHGRQRFNDNLATLLQVFLQRQPQPCCLVAHNGDRYDFPLLQAELASLSVISPLDGTFCVDSIAALKTLEQASSPSEHGPRKSYSLGSIYTRLYGQAPTDSHTAEGDVLALLSICQWKPQALLQWVDKHARPFSTIKPMYGMAATTGTASPRLCAATTSSPLATANLSPSNGRSRGKRPTSPPPENVPEAPSREGLLAPLGLLTFLTLAIAVLYGIFLASPGQ

SEQ ID NO: 150 - protein, 829 aa, Homo sapiens
  Human DNA2 AAH63664.1
FAIPASRMEQLNELELLMEKSFWEEAELPAELFQKKVVASFPRTVLSTGMDNRYLVLAVNTVQNKEGNCEKRLVITASQSLENKELCILRNDWCSVPVEPGDIIHLEGDCTSDTWIIDKDFGYLILYPDMLISGTSIASSIRCMRRAVLSETFRSSDPATRQMLIGTVLHEVFQKAINNSFAPEKLQELAFQTIQEIRHLKEMYRLNLSQDEIKQEVEDYLPSFCKWAGDFMHKNTSTDFPQMQLSLPSDNSKDNSTCNIEVVKPMDIEESIWSPRFGLKGKIDVTVGVKIHRGYKTKYKIMPLELKTGKESNSIEHRSQVVLYTLLSQERRADPEAGLLLYLKTGQMYPVPANHLDKRELLKLRNQMAFSLFHRISKSATRQKTQLASLPQIIEEEKTCKYCSQIGNCALYSRAVEQQMDCSSVPIVMLPKIEEETQHLKQTHLEYFSLWCLMLTLESQSKDNKKNHQNIWLMPASEMEKSGSCIGNLIRMEHVKIVCDGQYLHNFQCKHGAIPVTNLMAGDRVIVSGEERSLFALSRGYVKEINMTTVTCLLDRNLSVLPESTLFRLDQEEKNCDIDTPLGNLSKLMENTFVSKKLRDLIIDFREPQFISYLSSVLPHDAKDTVACILKGLNKPQRQAMKKVLLSKDYTLIVGMPGTGKTTTICTLVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLIIDLPSREHESLCHILGDFQRE

SEQ ID NO: 151 - protein, 1522 aa, Saccharomyces cerevisiae
  DNA2YEAST  P38859.1
MPGTPQKNKRSASISVSPAKKTEEKEIIQNDSKAILSKQTKRKKKYAFAPINNLNGKNTKVSNASVLKSIAVSQVRNTSRTKDINKAVSKSVKQLPNSQVKPKREMSNLSRHHDFTQDEDGPMEEVIWKYSPLQRDMSDKTTSAAEYSDDYEDVQNPSSTPIVPNRLKTVLSFTNIQVPNADVNQLIQENGNEQVRPKPAEISTRESLRNIDDILDDIEGDLTIKPTITKFSDLPSSPIKAPNVEKKAEVNAEEVDKMDSTGDSNDGDDSLIDILTQKYVEKRKSESQITIQGNTNQKSGAQESCGKNDNTKSRGEIEDHENVDNQAKTGNAFYENEEDSNCQRIKKNEKIEYNSSDEFSDDSLIELLNETQTQVEPNTIEQDLDKVEKMVSDDLRIATDSTLSAYALRAKSGAPRDGVVRLVIVSLRSVELPKIGTQKILECIDGKGEQSSVVVRHPWVYLEFEVGDVIHIIEGKNIENKRLLSDDKNPKTQLANDNLLVLNPDVLFSATSVGSSVGCLRRSILQMQFQDPRGEPSLVMTLGNIVHELLQDSIKYKLSHNKISMEIIIQKLDSLLETYSFSIIICNEEIQYVKELVMKEHAENILYFVNKFVSKSNYGCYTSISGTRRTQPISISNVIDIEENIWSPIYGLKGFLDATVEANVENNKKHIVPLEVKTGKSRSVSYEVQGLIYTLLLNDRYEIPIEFFLLYFTRDKNMTKFPSVLHSIKHILMSRNRMSMNFKHQLQEVFGQAQSRFELPPLLRDSSCDSCFIKESCMVLNKLLEDGTPEESGLVEGEFEILTNHLSQNLANYKEFFTKYNDLITKEESSITCVNKELFLLDGSTRESRSGRCLSGLVVSEVVEHEKTEGAYIYCFSRRRNDNNSQSMLSSQIAANDFVIISDEEGHFCLCQGRVQFINPAKIGISVKRKLLNNRLLDKEKGVTTIQSVVESELEQSSLIATQNLVTYRIDKNDIQQSLSLARFNLLSLFLPAVSPGVDIVDERSKLCRKTKRSDGGNEILRSLLVDNRAPKFRDANDDPVIPYKLSKDTTLNLNQKEAIDKVMRAEDYALILGMPGTGKTTVIAEIIKILVSEGKRVLLTSYTHSAVDNILIKLRNTNISIMRLGMKHKVHPDTQKYVPNYASVKSYNDYLSKINSTSVVATTCLGINDILFTLNEKDFDYVILDEASQISMPVALGPLRYGNRFIMVGDHYQLPPLVKNDAARLGGLEESLFKTFCEKHPESVAELTLQYRMCGDIVTLSNFLIYDNKLKCGNNEVFAQSLELPMPEALSRYRNESANSKQWLEDILEPTRKVVFLNYDNCPDIIEQSEKDNITNHGEAELTLQCVEGMLLSGVPCEDIGVMTLYRAQLRLLKKIFNKNVYDGLEILTADQFQGRDKKCIIISMVRRNSQLNGGALLKELRRVNVAMTRAKSKLIIIGSKSTIGSVPEIKSFVNLLEERNWVYTMCKDALYKYKFPDRSNAIDEARKGCGKRTGAKPITSKSKFVSDKPIIKEILQEYES

SEQ ID NO: 152 - protein, 490 aa, Human herpesvirus 2
  VP16 AAA45863.1
MDLLVDDLFADRDGVSPPPPRPAGGPKNTPAAPPLYATGRLSQAQLMPSPPMPVPPAALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAREESYRTVLANFCSALYRYLRASVRQLHRQAHMRGRNRDLREMLRTTIADRYYRETARLARVLFLHLYLFLSREILWAAYAEQMMRPDLFDGLCCDLESWRQLACLFQPLMFINGSLTVRGVPVEARRLRELNHIREHLNLPLVRSAAAEEPGAPLTTPPVLQGNQARSSGYFMLLIRAKLDSYSSVATSEGESVMREHAYSRGRTRNNYGSTIEGLLDLPDDDDAPAEAGLVAPRMSFLSAGQRPRRLSTTAPITDVSLGDELRLDGEEVDMTPADALDDFDLEMLGDVESPSPGMTHDPVSYGALDVDDFEFEQMFTDAMGIDDFGG

SEQ ID NO: 153 - DNA, 6101 bp, Artificial
  pCLS2690
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattccgtcgaccatggccaataccaaatataacgaagagttcctgctgtacctggccggctttgtggacgctgacggtagcatcatcgctcagattaaaccaagacagtctcggaagtttaaacatgagctaagcttgacctttgatgtgactcaaaagacccagcgccgttggtttctggacaagctagtggatgaaattggcgttggttacgtatatgattctggatccgtttcctattaccagttaagcgaaatcaagccgctgcacaacttcctgactcaactgcagccgtttctggaactgaaacagaaacaggcaaacctggttctgaaaattatcgaacagctgccgtctgcaaaagaatccccggccaaattcctggaagtttgtacctgggtggatcagattgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggatggtgatggctccatcattgctcagataaaaccacgtcaagggtataagttcaaacaccagctctccttgacttttcaggtcactcagaagacacaaagaaggtggttcttggacaaattggttgatcgtattggtgtgggctatgtcgctgaccgtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtccctggacaagtttcttgaagtgtgtacttgggtggatcagattgctgccttgaatgactccaagaccagaaaaaccacctctgagactgtgagggcagttctggatagcctctctgagaagaaaaagtcctctccttagccatggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 154 - DNA, 5885 bp, Artificial
  pCLS7673
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatgggttccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcggccgactgactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 155 - DNA, 22 bp, Artificial
  GS target sequence
tgccccagggtgagaaagtcca

SEQ ID NO: 156 - DNA, 6089 bp, Artificial
  pCLS2222
atggccaataccaaatataacgaagagttcctgctgtacctggccggctttgtggacggtgacggtagcatcatcgctcagattaatccaaaccagtcttctaagtttaaacatcgtctacgtttgaccttttatgtgactcaaaagacccagcgccgttggtttctggacaaactagtggatgaaattggcgttggttacgtacgtgattctggatccgtttcccagtacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgggaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggattctgatggctccatcattgctcagataaaaccacgtcaatctaacaagttcaaacaccagctctccttgacttttgcagtcactcagaagacacaaagaaggtggttcttggacaaattggttgataggattggtgtgggctatgtctatgacagtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttagtctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgacc

SEQ ID NO: 157 - DNA, 6220 bp, Artificial
  pCLS2510
tcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgccgaattcatggccaataccaaatataacgaagagttcctgctgtacctggccggctttgtggacggtgacggtagcatcatcgctcagattaaaccaaatcagtctcataagtttaaacatgctctacagttgacctttaaggtgactcaaaagacccagcgccgttggtttctggacaaactagtggatgaaattggcgttggttacgtacaggatagtggatccgtttccaactacatcttaagcgaaatcaagccgctgcacaacttcctgactcaactgcagccgtttctggaactgaaacagaaacaggcaaacctggccctgaaaattatcgaacagctgccgtctgcaaaagaatccccggacaaattcctggaagtttgtacctgggtggatcaggttgcagctctgaacgattctaagacgcgtaaaaccacttctgaaaccgttcgtgctgtgctggacagcctgagcgagaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggattctgatggctccatcattgctcagataaaaccaaatcaatctcacaagttcaaacaccagctctccttggcctttcaagtcactcagaagacacaaagaaggtggttcttggacaaattggttgataggattggtgtgggctatgtcagagacagaggctctgtgtcagactacatcctgtctaaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttagagatc

SEQ ID NO: 158 - DNA, 6233 bp, Artificial
  pCLS6163
taactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacgaagagttcctgctgtacctggccggctttgtggacggtgacggtagcatcgttgctcagattaaaccaaaccagcgtgctaagtttaaacatcagctaagcttgacctttcaggtgactcaaaagacccagcgccgttggctgctggacaaactagtggatgaaattggcgttggttacgtacaggattctggtagcgtttccaactaccgtttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggctgatcagattgcagctctgaacgattctaagacgcgtaaaaccacttctgaaaccgttcgtgctgtgctggacagcctgagcgagaagaagaaaccgtccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccacgtcaatcttacaagttcaaacaccagctccgtttgaccttttacgtcactcagaagacacaaagaaggtggttcttggacaaattggttgatcgtattggtgtgggctatgtcgaagactctggctctgtgtcacgttacgttctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttag

SEQ ID NO: 159 - DNA, 11446 bp, Artificial
  pCLS6810
tcatggtcatagctgtttcctgtgtgaaattgttatccgctcacaattccacacaacatacaggatccactagcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccttaggcgcgcctcgcgagtttaaaccgccaccatggccgcttatccttatgacgttcctgattacgctggatttatagctgccccagggtgagaaagtccaaggaggctccggatccggcggttctggatccggcggttctggttccgccgctagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaacccggggagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgacccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaa

SEQ ID NO: 160 - DNA, 64 bp, Artificial
  GS Locus specific forward primer
  misc_feature 31..40: n is a or c or t or g
ccatctcatccctgcgtgtctccgactcagnnnnnnnnnngctctctggctaactagagaaccc

SEQ ID NO: 161 - DNA, 53 bp, Artificial
  GS Locus specific reverse primer
cctatcccctgtgtgccttggcagtctcagtcgatcagcacgggcacgatgcc

SEQ ID NO: 162 - DNA, 67 bp, Artificial
  RAG1 Locus specific forward primer
  misc_feature 31..40: n is a or c or t or g
ccatctcatccctgcgtgtctccgactcagnnnnnnnnnnggcaaagatgaatcaaagattctgtcc

SEQ ID NO: 163 - DNA, 62 bp, Artificial
  XPC4 Locus specific forward primer
  misc_feature 31..40: n is a or c or t or g
ccatctcatccctgcgtgtctccgactcagnnnnnnnnnnaagaggcaagaaaatgtgcagc

SEQ ID NO: 164 - DNA, 60 bp, Artificial
  CAPNS1 Locus specific forward primer
  misc_feature 31..40: n is a or c or t or g
ccatctcatccctgcgtgtctccgactcagnnnnnnnnnncgagtcagggcgggattaag

SEQ ID NO: 165 - DNA, 57 bp, Artificial
  RAG1 Locus specific reverse primer
cctatcccctgtgtgccttggcagtctcaggatctcacccggaacagcttaaatttc

SEQ ID NO: 166 - DNA, 54 bp, Artificial
  XPC4 Locus specific reverse primer
cctatcccctgtgtgccttggcagtctcaggctgggcatatataaggtgctcaa

SEQ ID NO: 167 - DNA, 50 bp, Artificial
  CAPNS1 Locus specific reverse primer
cctatcccctgtgtgccttggcagtctcagcgagacttcacggtttcgcc

SEQ ID NO: 168 - skipped

SEQ ID NO: 169 - protein, 5 aa, Artificial
  GS stretch 1
GGGGS

SEQ ID NO: 170 - protein, 10 aa, Artificial
  GS stretch 2
GGGGSGGGGS

SEQ ID NO: 171 - protein, 595 aa, Artificial
  SC_GS-5-Trex
MANTKYNEEFLLYLAGFVDADGSIIAQIKPRQSRKFKHELSLTFDVTQKTQRRWFLDKLVDEIGVGYVYDSGSVSYYQLSEIKPLHNFLTQLQPFLELKQKQANLVLKIIEQLPSAKESPAKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPRQGYKFKHQLSLTFQVTQKTQRRWFLDKLVDRIGVGYVADRGSVSDYRLSEIKPLHNFLTQLQPFLKLKQKQANLVLKIIEQLPSAKESLDKFLEVCTWVDQIAALNDSKTRKTTSETVRAVLDSLSEKKKSSPGGGGSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEAAD

SEQ ID NO: 172 - protein, 600 aa, Artificial [sequence omitted]

SEQ ID NO: 173 - protein, 594 aa, Artificial
  Trex-5-SC_GS
MGSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEAGGGGSNTKYNEEFLLYLAGFVDADGSIIAQIKPRQSRKFKHELSLTFDVTQKTQRRWFLDKLVDEIGVGYVYDSGSVSYYQLSEIKPLHNFLTQLQPFLELKQKQANLVLKIIEQLPSAKESPAKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPRQGYKFKHQLSLTFQVTQKTQRRWFLDKLVDRIGVGYVADRGSVSDYRLSEIKPLHNFLTQLQPFLKLKQKQANLVLKIIEQLPSAKESLDKFLEVCTWVDQIAALNDSKTRKTTSETVRAVLDSLSEKKKSSP

SEQ ID NO: 174 - protein, 599 aa, Artificial
  Trex-10-SC_GS
MGSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEAGGGGSGGGGSNTKYNEEFLLYLAGFVDADGSIIAQIKPRQSRKFKHELSLTFDVTQKTQRRWFLDKLVDEIGVGYVYDSGSVSYYQLSEIKPLHNFLTQLQPFLELKQKQANLVLKIIEQLPSAKESPAKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPRQGYKFKHQLSLTFQVTQKTQRRWFLDKLVDRIGVGYVADRGSVSDYRLSEIKPLHNFLTQLQPFLKLKQKQANLVLKIIEQLPSAKESLDKFLEVCTWVDQIAALNDSKTRKTTSETVRAVLDSLSEKKKSSP

SEQ ID NO: 175 - DNA, 5672 bp, Artificial
  pCLS1853
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatggccaataccaaatataacaaagagttcctgctgtacctggccggctttgtggacggtgacggtagcatcatcgctcagattaaaccaaaccagtcttataagtttaaacatcagctaagcttgacctttcaggtgactcaaaagacccagcgccgttggtttctggacaaactagtggatgaaattggcgttggttacgtacgtgatcgcggatccgtttccaactacatcttaagcgaaatcaagccgctgcacaacttcctgactcaactgcagccgtttctgaaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccgactgataactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 176 - DNA, 20 bp, Artificial
  CMV forward primer
cgcaaatgggcggtaggcgt

SEQ ID NO: 177 - DNA, 24 bp, Artificial
  V5 reverse primer
cgtagaatcgagaccgaggagagg

SEQ ID NO: 178 - DNA, 33 bp, Artificial
  Link5TrexFor primer
ggaggtggaggttccgaggcaccccgggccgag

SEQ ID NO: 179 - DNA, 45 bp, Artificial
  Link5GSRev primer
tgcctcggaacctccacctccaggagaggactttttcttctcaga

SEQ ID NO: 180 - DNA, 42 bp, Artificial
  Link10TrexFor primer
ggcggatctggaggtggaggttccgaggcaccccgggccgag

SEQ ID NO: 181 - DNA, 51 bp, Artificial
  Link10GSRev primer
acctccacctccagatccgccacctccaggagaggactttttcttctcaga

SEQ ID NO: 182 - DNA, 39 bp, Artificial
  Link5GSFor primer
ggaggtggaggttccaataccaaatataacgaagagttc

SEQ ID NO: 183 - DNA, 45 bp, Artificial
  Link5TrexRev primer
ggtattggaacctccacctcccgcctccaggctggggtcatcagg

SEQ ID NO: 184 - DNA, 48 bp, Artificial
  Link10GSFor primer
ggaggttctggaggtggaggttccaataccaaatataacgaagagttc

SEQ ID NO: 185 - DNA, 51 bp, Artificial
  Link10TrexRev primer
acctccacctccagaacctccacctcccgcctccaggctggggtcatcagg

SEQ ID NO: 186 - DNA, 6867 bp, Artificial
  pCLS8082
ctcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattccgtcgaccatggccaataccaaatataacgaagagttcctgctgtacctggccggctttgtggacgctgacggtagcatcatcgctcagattaaaccaagacagtctcggaagtttaaacatgagctaagcttgacctttgatgtgactcaaaagacccagcgccgttggtttctggacaagctagtggatgaaattggcgttggttacgtatatgattctggatccgtttcctattaccagttaagcgaaatcaagccgctgcacaacttcctgactcaactgcagccgtttctggaactgaaacagaaacaggcaaacctggttctgaaaattatcgaacagctgccgtctgcaaaagaatccccggccaaattcctggaagtttgtacctgggtggatcagattgcagctctgaacgattctaagacgcgtaaaaccacttctgaaaccgttcgtgctgtgctggatagcctgagcgagaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggatggtgatggctccatcattgctcagataaaaccacgtcaagggtataagttcaaacaccagctctccttgacttttcaggtcactcagaagacacaaagaaggtggttcttggacaaattggttgatcgtattggtgtgggctatgtcgctgaccgtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtccctggacaagtttcttgaagtgtgtacttgggtggatcagattgctgccttgaatgactccaagaccagaaaaaccacctctgagactgtgagggcagttctggatagcctctctgagaagaaaaagtcctctcctggaggtggaggttccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcggccgactga

SEQ ID NO: 187 - DNA, 6882 bp, Artificial
  pCLS8052
ctcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattccgtcgaccatggccaataccaaatataacgaagagttcctgctgtacctggccggctttgtggacgctgacggtagcatcatcgctcagattaaaccaagacagtctcggaagtttaaacatgagctaagcttgacctttgatgtgactcaaaagacccagcgccgttggtttctggacaagctagtggatgaaattggcgttggttacgtatatgattctggatccgtttcctattaccagttaagcgaaatcaagccgctgcacaacttcctgactcaactgcagccgtttctggaactgaaacagaaacaggcaaacctggttctgaaaattatcgaacagctgccgtctgcaaaagaatccccggccaaattcctggaagtttgtacctgggtggatcagattgcagctctgaacgattctaagacgcgtaaaaccacttctgaaaccgttcgtgctgtgctggatagcctgagcgagaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggatggtgatggctccatcattgctcagataaaaccacgtcaagggtataagttcaaacaccagctctccttgacttttcaggtcactcagaagacacaaagaaggtggttcttggacaaattggttgatcgtattggtgtgggctatgtcgctgaccgtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtccctggacaagtttcttgaagtgtgtacttgggtggatcagattgctgccttgaatgactccaagaccagaaaaaccacctctgagactgtgagggcagttctggatagcctctctgagaagaaaaagtcctctcctggaggtggcggatctggaggtggaggttccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcggccgactga

SEQ ID NO: 188 - DNA, 6907 bp, Artificial
  pCLS8053
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatgggttccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcgggaggtggaggttccaataccaaatataacgaagagttcctgctgtacctggccggctttgtggacgctgacggtagcatcatcgctcagattaaaccaagacagtctcggaagtttaaacatgagctaagcttgacctttgatgtgactcaaaagacccagcgccgttggtttctggacaagctagtggatgaaattggcgttggttacgtatatgattctggatccgtttcctattaccagttaagcgaaatcaagccgctgcacaacttcctgactcaactgcagccgtttctggaactgaaacagaaacaggcaaacctggttctgaaaattatcgaacagctgccgtctgcaaaagaatccccggccaaattcctggaagtttgtacctgggtggatcagattgcagctctgaacgattctaagacgcgtaaaaccacttctgaaaccgttcgtgctgtgctggatagcctgagcgagaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggatggtgatggctccatcattgctcagataaaaccacgtcaagggtataagttcaaacaccagctctccttgacttttcaggtcactcagaagacacaaagaaggtggttcttggacaaattggttgatcgtattggtgtgggctatgtcgctgaccgtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtccctggacaagtttcttgaagtgtgtacttgggtggatcagattgctgccttgaatgactccaagaccagaaaaaccacctctgagactgtgagggcagttctggatagcctctctgagaagaaaaagtcctctccttagccatggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 189 - DNA, 6922 bp, Artificial
  pCLS8054
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcacaagtttgtacaaaaaagcaggctggcgcgcctacacagcggccttgccaccatgggttccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcgggaggtggaggttctggaggtggaggttccaataccaaatataacgaagagttcctgctgtacctggccggctttgtggacgctgacggtagcatcatcgctcagattaaaccaagacagtctcggaagtttaaacatgagctaagcttgacttttgatgtgactcaaaagacccagcgccgttggtttctggacaagctagtggatgaaattggcgttggttacgtatatgattctggatccgtttcctattaccagttaagcgaaatcaagccgctgcacaacttcctgactcaactgcagccgtttctggaactgaaacagaaacaggcaaacctggttctgaaaattatcgaacagctgccgtctgcaaaagaatccccggccaaattcctggaagtttgtacctgggtggatcagattgcagctctgaacgattctaagacgcgtaaaaccacttctgaaaccgttcgtgctgtgctggatagcctgagcgagaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggatggtgatggctccatcattgctcagataaaaccacgtcaagggtataagttcaaacaccagctctccttgacttttcaggtcactcagaagacacaaagaaggtggttcttggacaaattggttgatcgtattggtgtgggctatgtcgctgaccgtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtccctggacaagtttcttgaagtgtgtacttgggtggatcagattgctgccttgaatgactccaagaccagaaaaaccacctctgagactgtgagggcagttctggatagcctctctgagaagaaaaagtcctctccttagccatggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 190 - protein, 354 aa, Artificial
  SC_XPC4 protein
MANTKYNEEFLLYLAGFVDGDGSIIAQIKPNQSHKFKHALQLTFKVTQKTQRRWFLDKLVDEIGVGYVQDSGSVSNYILSEIKPLHNFLTQLQPFLELKQKQANLALKIIEQLPSAKESPDKFLEVCTWVDQVAALNDSKTRKTTSETVRAVLDSLSEKKKSSPAAGGSDKYNQALSKYNQALSKYNQALSGGGGSNKKFLLYLAGFVDSDGSIIAQIKPNQSHKFKHQLSLAFQVTQKTQRRWFLDKLVDRIGVGYVRDRGSVSDYILSKIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 191 - DNA, 2686 bp, Artificial
  pCLS0002
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 192 - protein, 354 aa, Artificial
  SC_CAPNS1 protein
MANTKYNEEFLLYLAGFVDGDGSIVAQIKPNQRAKFKHQLSLTFQVTQKTQRRWLLDKLVDEIGVGYVQDSGSVSNYRLSEIKPLHNFLTQLQPFLELKQKQANLVLKIIEQLPSAKESPDKFLEVCTWADQIAALNDSKTRKTTSETVRAVLDSLSEKKKPSPAAGGSDKYNQALSKYNQALSKYNQALSGGGGSNKKFLLYLAGFVDSDGSIIAQIKPRQSYKFKHQLRLTFYVTQKTQRRWFLDKLVDRIGVGYVEDSGSVSRYVLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 193 - protein, 354 aa, Artificial
  SC_GS protein
MANTKYNEEFLLYLAGFVDADGSIIAQIKPRQSRKFKHELSLTFDVTQKTQRRWFLDKLVDEIGVGYVYDSGSVSYYQLSEIKPLHNFLTQLQPFLELKQKQANLVLKIIEQLPSAKESPAKFLEVCTWVDQIAALNDSKTRKTTSETVRAVLDSLSEKKKSSPAAGGSDKYNQALSKYNQALSKYNQALSGGGGSNKKFLLYLAGFVDGDGSIIAQIKPRQGYKFKHQLSLTFQVTQKTQRRWFLDKLVDRIGVGYVADRGSVSDYRLSEIKPLHNFLTQLQPFLKLKQKQANLVLKIIEQLPSAKESLDKFLEVCTWVDQIAALNDSKTRKTTSETVRAVLDSLSEKKKSSP

SEQ ID NO: 194 - protein, 236 aa, Artificial
  Trex2 (236 aa)
MSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 195 - protein, 167 aa, Artificial; identical to SEQ ID NO: 76
  I-CreI

SEQ ID NO: 196 - DNA, 6969 bp, Artificial
  pCLS8518
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgaattcgactcactgttgggagacccaagctggctagttaagctatcaacaagtttgtacaaaaaagcaggctggcgcgcctacacagcggccttgccaccatgggttccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcgggaggtggaggttctggaggtggaggttccaataccaaatataacgaagagttcctgctgtacctggccggctttgtggacggtgacggtagcatcgttgctcagattaaaccaaaccagcgtgctaagtttaaacatcagctaagcttgacctttcaggtgactcaaaagacccagcgccgttggctgctggacaaactagtggatgaaattggcgttggttacgtacaggattctggtagcgtttccaactaccgtttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggctgatcagattgcagctctgaacgattctaagacgcgtaaaaccacttctgaaaccgttcgtgctgtgctggacagcctgagcgagaagaagaaaccgtccccggcggccggtggatctgataagtataatcaggctctgtctaaatacaaccaagcactgtccaagtacaatcaggccctgtctggtggaggcggttccaacaaaaaattcctgctgtatcttgctggatttgtggattctgatggctccatcattgctcagataaaaccacgtcaatcttacaagttcaaacaccagctccgtttgaccttttacgtcactcagaagacacaaagaaggtggttcttggacaaattggttgatcgtattggtgtgggctatgtcgaagactctggctctgtgtcacgttacgttctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcaggttgctgccttgaatgactccaagaccagaaaaaccacctctgagactgtgagggcagttctggatagcctctctgagaagaaaaagtcctctccttagtaactcgagcgctagcacccagctttcttgtacaaagtggtgatctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 197 - protein, 599 aa, Artificial
  Trex-SC_CAPNS1 protein
MGSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEAGGGGSGGGGSNTKYNEEFLLYLAGFVDGDGSIVAQIKPNQRAKFKHQLSLTFQVTQKTQRRWLLDKLVDEIGVGYVQDSGSVSNYRLSEIKPLHNFLTQLQPFLELKQKQANLVLKIIEQLPSAKESPDKFLEVCTWADQIAALNDSKTRKTTSETVRAVLDSLSEKKKPSPAAGGSDKYNQALSKYNQALSKYNQALSGGGGSNKKFLLYLAGFVDSDGSIIAQIKPRQSYKFKHQLRLTFYVTQKTQRRWFLDKLVDRIGVGYVEDSGSVSRYVLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 198 - skipped

SEQ ID NO: 199 - DNA, 56 bp, Artificial
  synthetic DNA
  misc_feature (27)..(36)
  misc_feature (27)..(36): n is a, c, g, or t
ccatctcatccctgcgtgtctccgacnnnnnnnnnncgagtcagggcgggattaag

SEQ ID NO: 200 - DNA, 50 bp, Artificial; identical to SEQ ID NO: 167
  CAPNS1 locus specific reverse primer

SEQ ID NO: 201 - protein, 508 aa, Homo sapiens
  Human Tdt protein
MDPPRASHLSPRKKRPRQTGALMASSPQDIKFQDLVVFILEKKMGTTRRAFLMELARRKGFRVENELSDSVTHIVAENNSGSDVLEWLQAQKVQVSSQPELLDVSWLIECIRAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRFERDLRRYATHERKMILDNHALYDKTKRIFLKAESEEEIFAHLGLDYIEPWERNA

SEQ ID NO: 202 - DNA, 6438 bp, Artificial
  pCLS3841
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgcgaattcagatctggtaccggtccggaattcccgggatatcgtcgacccacgcgtccgcaccaccagatgggccagccagaggcagcagcagcctcttcccatggatccaccacgagcgtcccacttgagccctcggaagaagagaccccggcagacgggtgccttgatggcctcctctcctcaagacatcaaatttcaagatttggtcgtcttcattttggagaagaaaatgggaaccacccgcagagcgttcctcatggagctggcccgcaggaaagggttcagggttgaaaatgagctcagtgattctgtcacccacattgtagcagagaacaactcgggttcggatgttctggagtggcttcaagcacagaaagtacaagtcagctcacaaccagagctcctcgatgtctcctggctgatcgaatgcataggagcagggaaaccggtggaaatgacaggaaaacaccagcttgttgtgagaagagactattcagatagcaccaacccaggccccccgaagactccaccaattgctgtacaaaagatctcccagtatgcgtgtcagagaagaaccactttaaacaactgtaaccagatattcacggatgcctttgatatactggctgaaaactgtgagtttagagaaaatgaagactcctgtgtgacatttatgagagcagcttctgtattgaaatctctgccattcacaatcatcagtatgaaggacacagaaggaattccctgcctggggtccaaggtgaagggtatcatagaggagattattgaagatggagaaagttctgaagttaaagctgtgttaaatgatgaacgatatcaatccttcaaactctttacttctgtatttggagtggggctgaagacttctgagaagtggttcaggatgggtttcagaactctgagtaaagtaaggtcggacaaaagcctgaaatttacacgaatgcagaaagcaggatttctgtattatgaagaccttgtcagctgtgtgaccagggcagaagcagaggccgtcagtgtgctggttaaagaggctgtctgggcatttcttccggatgctttcgtcaccatgacaggagggttccggaggggtaagaagatggggcatgatgtagattttttaattaccagcccaggatcaacagaggatgaagagcaacttttacagaaagtgatgaacttatgggaaaagaagggattacttttatattatgaccttgtggagtcaacatttgaaaagctcaggttgcctagcaggaaggttgatgctttggatcattttcaaaagtgctttctgattttcaaattgcctcgtcaaagagtggacagtgaccagtccagctggcaggaaggaaagacctggaaggccatccgtgtggatttagttctgtgcccctacgagcgtcgtgcctttgccctgttgggatggactggctcccggcagtttgagagagacctccggcgctatgccacacatgagcggaagatgattctggataaccatgctttatatgacaagaccaagaggatattcctcaaagcagaaagtgaagaagaaatttttgcgcatctgggattggattatattgaaccgtgggaaagaaatgcctaggaaagtgttgtcaacatttttttcctattcttttcaagttaaataaattatgcttcatattagtaaaagatgccataggagagtttggggttatttaggtcttattgaaatgcagattgctactagaaataaataactttggaaacatgggaaggtgccactggtaatgggtaaggttctaataggccatgtttatgactgttgcatagaattcacaatgcatttttcaagagaaatgatgttgtcactggtggctcattcagggaagctcatcaaagcccactttgttcgcagtgtagctgaaatactgtctatctctaataaaaacaggaggaaacaaaaaaaaaaaaaaaaaaaaaaaaaaaaaaaaagggcggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatatt

SEQ ID NO: 203 - DNA, 48 bp, Artificial
  LinkTDTFor primer
ggcggatctggaggtggaggttccgatccaccacgagcgtcccacttg

SEQ ID NO: 204 - DNA, 29 bp, Artificial
  TDTRev
ggctcgagctaggcatttctttcccacgg

SEQ ID NO: 205 - DNA, 30 bp, Artificial
  TDTFor
ggcgcgccatggatccaccacgagcgtccc

SEQ ID NO: 206 - DNA, 48 bp, Artificial
  Link10TDTRev
acctccacctccagaacctccacctccggcatttctttcccacggttc

SEQ ID NO: 207 - DNA, 24 bp, Artificial
  N1 meganuclease target sequence
ttgttctcaggtacctcagccagc

SEQ ID NO: 208 - DNA, 49 bp, Artificial
  N2 TALEN target sequence
tatatttaagcacttatatgtgtgtaacaggtataagtaaccataaaca

SEQ ID NO: 209 - protein, 1065 aa, Artificial
  N2 TALEN monomer 1 protein
MADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 210 - protein, 1065 aa, Artificial
  N2 TALEN monomer 2 protein
MADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 211 - DNA, 8083 bp, Artificial
  pCLS8964
gtttgtttaaacttggtaccataactagttcggcgcgccactagcgctgtcacgcgtctccatggccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctaggaaagcggccgcggagctccaggaattctgcagatcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgcgccaaagctaactgtaggactgagtctattctaaactgaaagcctggacatctggagtaccagggggagatgacgtgttacgggcttccataaaagcagctggctttgaatggaaggagccaagaggccagcacaggagcggattcgtcgctttcacggccatcgagccgaacctctcgcaagtccgtgagccgttaaggaggcccccagtcccgacccttcgccccaagcccctcggggtccccgggcctggtactccttgccacacgggaggggcgcggaagccggggcggaggaggagccaaccccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgg

SEQ ID NO: 212 - DNA, 8083 bp, Artificial
  pCLS8965
gtttgtttaaacttggtaccataactagttcggcgcgccactagcgctgtcacgcgtctccatggccgaccccattcgttcgcgcacaccaagtcctgcccgcgagcttctgcccggaccccaacccgatggggttcagccgactgcagatcgtggggtgtctccgcctgccggcggccccctggatggcttgccggctcggcggacgatgtcccggacccggctgccatctccccctgccccctcacctgcgttctcggcgggcagcttcagtgacctgttacgtcagttcgatccgtcactttttaatacatcgctttttgattcattgcctcccttcggcgctcaccatacagaggctgccacaggcgagtgggatgaggtgcaatcgggtctgcgggcagccgacgcccccccacccaccatgcgcgtggctgtcactgccgcgcggcccccgcgcgccaagccggcgccgcgacgacgtgctgcgcaaccctccgacgcttcgccggcggcgcaggtggatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagcgatcctctaggaaagcggccgcggagctccaggaattctgcagatcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgcgccaaagctaactgtaggactgagtctattctaaactgaaagcctggacatctggagtaccagggggagatgacgtgttacgggcttccataaaagcagctggctttgaatggaaggagccaagaggccagcacaggagcggattcgtcgctttcacggccatcgagccgaacctctcgcaagtccgtgagccgttaaggaggcccccagtcccgacccttcgccccaagcccctcggggtccccgggcctggtactccttgccacacgggaggggcgcggaagccggggcggaggaggagccaaccccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgg

SEQ ID NO: 213 - DNA, 5428 bp, Artificial
  pCLS0003
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 214 - DNA, 57 bp, Artificial
  F_T2 primer
ccatctcatccctgcgtgtctccgactcagtagctttacatttactgaacaaataac

SEQ ID NO: 215 - DNA, 57 bp, Artificial; identical to SEQ ID NO: 165
  R_T1 primer

SEQ ID NO: 216 - protein, 354 aa, Artificial; identical to SEQ ID NO: 58
  SC_RAG

SEQ ID NO: 217 - DNA, 27 bp, Artificial sequence
  Table 8 sequence example 1
attgttctcaggcgtacctcagccagc

SEQ ID NO: 218 - DNA, 27 bp, Artificial sequence
  Table 8 sequence example 2
attgttctcaggtacatctcagccagc

SEQ ID NO: 219 - DNA, 27 bp, Artificial sequence
  Table 8 sequence example 3
attgttctcaggtacccctcagccagc

SEQ ID NO: 220 - DNA, 28 bp, Artificial sequence
  Table 8 sequence example 4
attgttctcaggtacgggctcagccagc

SEQ ID NO: 221 - DNA, 28 bp, Artificial sequence
  Table 8 sequence example 5
attgttctcagggcgtacctcagccagc

SEQ ID NO: 222 - DNA, 28 bp, Artificial sequence
  Table 8 sequence example 6
attgttctcaggtacagtctcagccagc

SEQ ID NO: 223 - DNA, 28 bp, Artificial sequence
  Table 8 sequence example 7
attgttctcaggtacggggctcagccag

SEQ ID NO: 224 - DNA, 29 bp, Artificial sequence
  Table 8 sequence example 8
attgttctcagacccgtacctcagccagc

SEQ ID NO: 225 - DNA, 29 bp, Artificial sequence
  Table 8 sequence example 9
attgttctcagcctcgtacctcagccagc

SEQ ID NO: 226 - DNA, 29 bp, Artificial sequence
  Table 8 sequence example 10
attgttctcagcttcgtacctcagccagc

SEQ ID NO: 227 - DNA, 29 bp, Artificial sequence
  Table 8 sequence example 11
attgttctcaggtactggactcagccagc

SEQ ID NO: 228 - DNA, 29 bp, Artificial sequence
  Table 8 sequence example 12
attgttctcaggtacagggctcagccagc

SEQ ID NO: 229 - DNA, 30 bp, Artificial sequence
  Table 8 sequence example 13
attgttctcaggtacgggaactcagccagc

SEQ ID NO: 230 - DNA, 30 bp, Artificial sequence
  Table 8 sequence example 14
attgttctcaggtacgaaggctcagccagc

SEQ ID NO: 231 - DNA, 30 bp, Artificial sequence
  Table 8 sequence example 15
attgttctcagttcctgtacctcagccagc

SEQ ID NO: 232 - DNA, 31 bp, Artificial sequence
  Table 8 sequence example 16
attgttctcaggtacgggtggctcagccagc

SEQ ID NO: 233 - DNA, 31 bp, Artificial sequence
  Table 8 sequence example 17
attgttctcaggtactggttactcagccagc

SEQ ID NO: 234 - DNA, 31 bp, Artificial sequence
  Table 8 sequence example 18
attgttctcaggtacccatacctcagccagc

SEQ ID NO: 235 - DNA, 32 bp, Artificial sequence
  Table 8 sequence example 19
attgttctcaggttacctgtacctcagccagc

SEQ ID NO: 236 - DNA, 32 bp, Artificial sequence
  Table 8 sequence example 20
attgttctcaggtacaagggggctcagccagc

SEQ ID NO: 237 - DNA, 33 bp, Artificial sequence
  Table 8 sequence example 21
attgttctcagggccgcccgtacctcagccagc

SEQ ID NO: 238 - DNA, 26 bp, Artificial sequence
  Table 9 sequence example 1
cagggccgcggtgcgcagtgtccgac

SEQ ID NO: 239 - DNA, 26 bp, Artificial sequence
  Table 9 sequence example 2
cagggccgcgccgtgcagtgtccgac

SEQ ID NO: 240 - DNA, 26 bp, Artificial sequence
  Table 9 sequence example 3
cagggccgcggcgtgcagtgtccgac

SEQ ID NO: 241 - DNA, 26 bp, Artificial sequence
  Table 9 sequence example 4
cagggccgcggtgcacagtgtccgac

SEQ ID NO: 242 - DNA, 27 bp, Artificial sequence
  Table 9 sequence example 5
cagggccgcggccgtgcagtgtccgac

SEQ ID NO: 243 - DNA, 27 bp, Artificial sequence
  Table 9 sequence example 6
cagggccgcggtgctgcagtgtccgac

SEQ ID NO: 244 - DNA, 27 bp, Artificial sequence
  Table 9 sequence example 7
cagggccgcgcctgtgcagtgtccgac

SEQ ID NO: 245 - DNA, 28 bp, Artificial sequence
  Table 9 sequence example 8
cagggccgcgttctgtgcagtgtccgac

SEQ ID NO: 246 - DNA, 28 bp, Artificial sequence
  Table 9 sequence example 9
cagggccgcggtgcgggcagtgtccgac

SEQ ID NO: 247 - DNA, 28 bp, Artificial sequence
  Table 9 sequence example 10
cagggccgcggtccgtgcagtgtccgac

SEQ ID NO: 248 - DNA, 28 bp, Artificial sequence
  Table 9 sequence example 11
cagggccgcggtgcaggcagtgtccgac

SEQ ID NO: 249 - DNA, 29 bp, Artificial sequence
  Table 9 sequence example 12
cagggccgcggtgcaaagcagtgtccgac

SEQ ID NO: 250 - DNA, 29 bp, Artificial sequence
  Table 9 sequence example 13
cagggccgcggtgcagtgcagtgtccgac

SEQ ID NO: 251 - DNA, 29 bp, Artificial sequence
  Table 9 sequence example 14
cagggccgcggtgcggtgcagtgtccgac

SEQ ID NO: 252 - DNA, 29 bp, Artificial sequence
  Table 9 sequence example 15
cagggccgcgtgtctgtgcagtgtccgac

SEQ ID NO: 253 - DNA, 30 bp, Artificial sequence
  Table 9 sequence example 16
cagggccgcggtgcaaggtcagtgtccgac

SEQ ID NO: 254 - DNA, 30 bp, Artificial sequence
  Table 9 sequence example 17
cagggccgcggtgcccgtgcagtgtccgac

SEQ ID NO: 255 - DNA, 30 bp, Artificial sequence
  Table 9 sequence example 18
cagggccgcggtgcaagtgcagtgtccgac

SEQ ID NO: 256 - DNA, 32 bp, Artificial sequence
  Table 9 sequence example 19
cagggccgcggtgcaagcagggagtgtccgac

(19) United States
(12) Patent Application Publication
(10) Pub. No.: US 2013/0337454 A1
(43) Pub. Date: Dec. 19, 2013

(54) METHOD FOR INCREASING THE EFFICIENCY OF DOUBLE-STRAND BREAK-INDUCED MUTAGENESIS

(71) Applicants: Philippe Duchateau, Draveil (FR); Alexandre Juillerat, Paris (FR); George H. Silva, Le Piessis Trevise (FR); Jean-Charles Epinat, Les Lilas (FR)

(72) Inventors: Philippe Duchateau, Draveil (FR); Alexandre Juillerat, Paris (FR); George H. Silva, Le Piessis Trevise (FR); Jean-Charles Epinat, Les Lilas (FR)

(21) Appl. No.: 13/880,860

(22) PCT Filed: Oct. 27, 2011
(86) PCT No.: PCT/US2011/058133

§ 371 Date: Jul. 24, 2013

Related U.S. Application Data
(60) Provisional application No. 61/407,339, filed on Oct. 27, 2010.
(60) Provisional application No. 61/472,072, filed on Apr. 5, 2011.
(60) Provisional application No. 61/505,783, filed on Jul. 8, 2011.

(51) Int. Cl. C12N 15/01; C12N 9/16

(52) U.S. Cl. CPC C12N 15/01; C12N 9/16
USPC 435/6.12; 435/188; 435/320.1; 536/23.2

(57) ABSTRACT

The present invention relates to a method for increasing double-strand break-induced mutagenesis at a genomic locus of interest in a cell, thereby giving new tools for genome engineering, including therapeutic applications and cell line engineering. More specifically, the present invention concerns a method for increasing double-strand break-induced mutagenesis at a genomic locus of interest, leading to a loss of genetic information and preventing any scarless re-ligation of said genomic locus of interest by NHEJ. The present invention also relates to engineered endonucleases, chimeric or not, vectors, compositions and kits used to implement this method.

### CROSS-REFERENCE TO RELATED APPLICATIONS

This application claims priority to U.S. Provisional Applications U.S. 61/407,339, filed Oct. 27, 2010, U.S. 61/472,072, filed Apr. 5, 2011 and U.S. 61/505,783, filed Jul. 8, 2011; each of which is incorporated by reference in its entirety.

### FIELD OF THE INVENTION

The present invention relates to a method for increasing double-strand break-induced mutagenesis at a genomic locus of interest in a cell, thereby providing new tools for genome engineering, including therapeutic applications and cell line engineering. More specifically, the present invention concerns a method for increasing double-strand break-induced mutagenesis at a genomic locus of interest, leading to a loss of genetic information and preventing any scarless re-ligation of said genomic locus of interest by NHEJ (non-homologous end joining). The present invention also relates to engineered endonucleases, chimeric or not, vectors, compositions and kits used to implement this method.

### BACKGROUND OF THE INVENTION

Mammalian genomes constantly suffer from various types of damage of which double-strand breaks (DSB) are considered the most dangerous (Haber 2000). For example, DSBs can arise when the replication fork encounters a nick or when ionizing radiation particles create clusters of reactive oxygen species along their path. These reactive oxygen species may in turn themselves cause DSBs. For cultured mammalian cells that are dividing, 5-10% appear to have at least one chromosomal break (or chromatid gap) at any one time (Lieber and Karanjawala 2004). Hence, the need to repair DSBs arises commonly (Li, Vogel et al. 2007) and is critical for cell survival (Haber 2000). Failure to correct or incorrect repair can result in deleterious genomic rearrangements, cell cycle arrest, and/or cell death.

Repair of DSBs can occur through diverse mechanisms that can depend on cellular context. Repair via homologous recombination, the most accurate process, is able to restore the original sequence at the break. Because of its strict dependence on extensive sequence homology, this mechanism is suggested to be active mainly during the S and G2 phases of the cell cycle where the sister chromatids are in close proximity (Sonoda, Hochegger et al. 2006). Single-strand annealing is another homology-dependent process that can repair a DSB between direct repeats and thereby promotes deletions (Paques and Haber 1999). Finally, non-homologous end joining (NHEJ) of DNA is a major pathway for the repair of DSBs because it can function throughout the cell cycle and because it does not require a homologous chromosome (Moore and Haber 1996).

NHEJ comprises at least two different processes (Feldmann, Schmiemann et al. 2000). The main and best characterized mechanism involves rejoining of what remains of the two DNA ends through direct re-ligation (Critchlow and Jackson 1998) or via the so-called microhomology-mediated end joining (MMEJ) (Ma, Kim et al. 2003). Although perfect re-ligation of the broken ends is probably the most frequent event, it could be accompanied by the loss or gain of several nucleotides.

Like most DNA repair processes, there are three enzymatic activities required for repair of DSBs by the NHEJ pathway: (i) nucleases to remove damaged DNA, (ii) polymerases to aid in the repair, and (iii) a ligase to restore the phosphodiester backbone. Depending on the nature of the DNA ends, DNA can be simply re-ligated or terminal nucleotides can be modified or removed by inherent enzymatic activities, such as phosphokinases and exo-nucleases. Missing nucleotides can also be added by polymerase μ or λ. In addition, an alternative or so-called back-up pathway has been described that does not depend on ligase IV and Ku components and has been involved in class switching and V(D)J recombination (Ma, Kim et al. 2003). Overall, NHEJ can be viewed as a flexible pathway for which the unique goal is to restore the chromosomal integrity, even at the cost of excision or insertion of nucleotide(s).

DNA repair can be triggered by both physical and chemical means. Several chemicals are known to cause DNA lesions and are used routinely. Radiomimetic agents, for example, work through free-radical attack on the sugar moieties of DNA (Povirk 1996). A second group of drugs that induce DNA damage includes inhibitors of topoisomerase I (TopoI) and II (TopoII) (Burden and N. 1998; Teicher 2008). Other classes of chemicals bind covalently to the DNA and form bulky adducts that are repaired by the nucleotide excision repair (NER) system (Nouspikel 2009). Chemicals inducing DNA damage have a diverse range of applications, however, although certain agents are more commonly applied in studying a particular repair pathway (e.g., cross-linking agents are favored for NER studies), most drugs simultaneously provoke a variety of lesions (Nagy and Soutoglou 2009). Furthermore, the overall yield of induced mutations using these classical strategies is quite low, and the DNA damage leading to mutagenesis cannot be targeted to a precise genomic DNA sequence.

The most widely used site-directed mutagenesis strategy is gene targeting (GT) via homologous recombination (HR). Efficient GT procedures in yeast and mouse have been available for more than 20 years (Capecchi 1989; Rothstein 1991). Successful GT has also been achieved in Arabidopsis and rice plants (Hanin, Volrath et al. 2001) (Terada, Urawa et al. 2002; Endo, Osakabe et al. 2006; Endo, Osakabe et al. 2007). Typically, GT events occur in a fairly small population of treated mammalian cells and is extremely low in higher plant cells, ranging between 0.01-0.1% of the total number of random integration events (Terada, Johzuka-Hisatomi et al. 2007). The low GT frequencies reported in various organisms are thought to result from competition between HR and NHEJ for repair of DSBs. As a consequence, the ends of a donor molecule are likely to be joined by NHEJ rather than participating in HR, thus reducing GT frequency. There are extensive data indicating that DSB repair by NHEJ is error-prone due to end-joining processes that generate insertions and/or deletions (Britt 1999). Thus, these NHEJ-based strategies might be more effective than HR-based strategies for targeted mutagenesis into cells.

Expression of I-SceI, a rare cutting endonuclease, has been shown to introduce mutations at I-SceI cleavage sites in Arabidopsis and tobacco (Kirik, Salomon et al. 2000). However, the use of endonucleases is limited to rarely occurring natural recognition sites or to artificially introduced target sites. To overcome this problem, meganucleases with engineered specificity towards a chosen sequence have been developed. Meganucleases show high specificity to their DNA target. These proteins being able to cleave a unique chromosomal sequence and therefore do not affect global genome integrity. Natural meganucleases are essentially represented by homing endonucleases, a widespread class of proteins found in eukaryotes, bacteria and archae (Chevalier and Stoddard 2001). Early studies of the I-SceI and HO homing endonucleases illustrated how the cleavage activity of these proteins can be used to initiate HR events in living cells and demonstrated the recombinogenic properties of chromosomal DSBs (Dujon, Colleaux et al. 1986; Haber 1995). Since then, meganuclease-induced HR has been successfully used for genome engineering purposes in bacteria (Posfai, Kolisnychenko et al. 1999), mammalian cells (Sargent, Brenneman et al. 1997; Cohen-Tannoudji, Robine et al. 1998; Donoho, Jasin et al. 1998), mice (Gouble, Smith et al. 2006) and plants (Puchta, Dujon et al. 1996; Siebert and Puchta 2002). Meganucleases have emerged as scaffolds of choice for deriving genome engineering tools cutting a desired target sequence (Paques and Duchateau 2007).

Combinatorial assembly processes allowing for the engineering of meganucleases with modified specificities have been described by Arnould et al. (Arnould, Chames et al. 2006; Arnould, Perez et al. 2007); Smith et al. (Smith, Grizot et al. 2006), Grizot et al. (Grizot, Smith et al. 2009). Briefly, these processes rely on the identification of locally engineered variants with a substrate specificity that differs from that of the wild-type meganuclease by only a few nucleotides. Another type of specific nucleases are the so-called Zinc-finger nucleases (ZFNs). ZFNs are chimeric proteins composed of a synthetic zinc-finger-based DNA binding domain fused to a DNA cleavage domain. By modification of the zinc-finger DNA binding domain, ZFNs can be specifically designed to cleave virtually any long stretch of dsDNA sequence (Kim, Cha et al. 1996; Cathomen and Joung 2008). A NHEJ-based targeted mutagenesis strategy was recently developed for several organisms by using synthetic ZFNs to generate DSBs at specific genomic sites (Lloyd, Plaisier et al. 2005; Beumer, Trautman et al. 2008; Doyon, McCammon et al. 2008; Meng, Noyes et al. 2008). Subsequent repair of the DSBs by NHEJ frequently produces deletions and/or insertions at the joining site. For example, in zebrafish embryos the injection of mRNA coding for engineered ZFNs led to animals carrying the desired heritable mutations (Doyon, McCammon et al. 2008). In plants, similar NHEJ-based targeted mutagenesis has also been successfully applied (Lloyd, Plaisier et al. 2005). Although these powerful tools are available, there is still a need to further improve double-strand break-induced mutagenesis.

The inventors have developed a new approach to increase the efficiency of targeted DSB-induced mutagenesis and have created a new type of meganucleases comprising several catalytic domains to implement this new approach. These novel enzymes allow a DNA cleavage that will lead to the loss of genetic information and any NHEJ pathway will produce targeted mutagenesis.

### BRIEF SUMMARY OF THE INVENTION

In one of its embodiments, the present invention relates to a method for increasing double-strand break-induced mutagenesis at a genomic locus of interest in a cell, thereby giving new tools for genome engineering, including therapeutic applications and cell line engineering. More specifically, in a first aspect, the present invention concerns a method for increasing double-strand break-induced mutagenesis at a genomic locus of interest, leading to a loss of genetic information and preventing any scarless re-ligation of said genomic locus of interest by NHEJ.

In a second aspect, the present invention relates to engineered enzymes and more particularly to chimeric rare-cutting endonucleases able to target a DNA sequence within a genomic locus of interest to generate at least one DNA double-strand break and a loss of genetic information around said DNA sequence thus preventing any scarless re-ligation of said genomic locus of interest by NHEJ.

In a third aspect, the present invention concerns a method for the generation of at least two-nearby DNA double-strand breaks at a genomic locus of interest to prevent any scarless re-ligation of said genomic locus of interest by NHEJ.

In a fourth aspect, the present invention relates to engineered enzymes and more particularly to engineered rare-cutting endonucleases, chimeric or not, able to target a DNA sequence within a genomic locus of interest to generate at said locus of interest at least two-nearby DNA double-strand breaks leading to at least the removal of a DNA fragment and thus preventing any scarless re-ligation of said genomic locus of interest by NHEJ. In a fifth aspect, the present invention describes a method to identify at a genomic locus of interest a DNA target sequence cleavable at least twice by a fusion protein leading at least to a loss of genetic information and preventing any scarless re-ligation of said genomic locus of interest by NHEJ.

In a sixth aspect, the present invention relates to fusion proteins able to generate at least two nearby DNA double-strand breaks into a genomic locus of interest comprising one DNA target sequence cleavable by one rare-cutting endonuclease nearby one DNA target sequence cleavable by one frequent-cutting endonuclease.

The present invention also relates to specific vectors, compositions and kits used to implement this method.

The above objects highlight certain aspects of the invention. Additional objects, aspects and embodiments of the invention are found in the following detailed description of the invention.

### BRIEF DESCRIPTION OF THE FIGURES

In addition to the preceding features, the invention further comprises other features which will emerge from the description which follows, as well as to the appended drawings. A more complete appreciation of the invention and many of the attendant advantages thereof will be readily obtained as the same becomes better understood by reference to the following Figures in conjunction with the detailed description below.

FIG. 1: Elimination of an intervening sequence enhances DSB-induced mutagenesis. The 22 bp DNA sequences recognized by D21m (or D21) and R1m (or R21), respectively, are introduced into a plasmid. A 10-bp intervening sequence is cloned between the two recognition sequences to avoid steric hindrance upon meganuclease binding. Introduction of the target plasmid within a cell, together with plasmids expressing the meganucleases D21m and R1m, results in the simultaneous cleavage of the two target sites. The intervening fragment comprising the 10-bp sequence surrounded by half of each target site is excised. Subsequent NHEJ, either via re-ligation of compatible or incompatible DNA ends, leads to mutagenic events since genetic information was lost.

FIG. 2: Schematic representation of the analyses performed to detect DSB-induced mutations. HEK293 cells are simultaneously transfected with target plasmid and either one or two different meganuclease expressing plasmids. DNA is extracted two days post transfection and specific PCR is performed. PCR products are analyzed using deep sequencing technology (454, Roche). Alternatively, a mutation detection assay (Transgenomic, Inc. USA) is performed. PCR product from untreated cells is mixed (equimolar) with PCR products treated with the meganucleases. The melting/annealing step generates heteroduplex DNA, recognized and cleaved by the CEL-1 enzyme. After digestion, DNA bands are resolved on an analytic gel and each band is quantified by densitometry.

FIG. 3: Sequence of the target DNA recognized by I-CreI. C1221 represents a palindromic DNA sequence recognized and cleaved by the I-CreI meganuclease. Nucleotides are numbered outward (−/+) from the center of the target. Nucleotides at positions −2 to +2 do not directly contact the protein but rather interfere with the cleavage activity of the protein. The table represents a subset of the tested targets with nucleotide substitution at positions −2 to +2. The binding and cleavage activity of I-CreI on the target is indicated (++, strong, +, good, +/−, weak; −, no activity). Activities were determined in vitro.

FIG. 4: Strategies to enhance DSB-induced mutagenesis. Loss of genetic information can be obtained by one or any variations of the following described strategies as illustrating examples (slight vertical lanes indicate specific DNA recognition domains):—simultaneous DSBs generated by two different specific rare-cutting endonucleases (A);—chimeric rare-cutting endonucleases with two endonucleases catalytic domains (bi-functional) (B);—chimeric rare-cutting endonucleases with one DNA-binding domain and two endonucleases catalytic domains (bi-functional) (C);—fusion protein between a rare-cutting endonuclease, a endonuclease catalytic domain and a frequent-cutting endonuclease (multi-functional) (D);—chimeric rare-cutting endonucleases with one exonuclease catalytic domains capable to process DNA ends (bi-functional) (E).

FIG. 5: Effect of Trex2 expression on SC_GS-induced mutagenic DSB repair. A: Percentage of GFP+ cells induced on NHEJ model after transfection of SC_GS (SEQ ID NO: 153) with empty vector (SEQ ID NO: 175) or with increasing amount of Trex2 expression vector (SEQ ID NO: 154). B: Percentage of mutagenesis (insertions and deletions) detected in the vicinity of the GS_CHO1 target present on the NHEJ model induced by either SC_GS (SEQ ID NO: 153) with empty vector (SEQ ID NO: 175) or with two different doses of Trex2 encoding vector (SEQ ID NO: 154). C: Percentage of events corresponding to a deletion of 2 (del2), 3 (del3) or 4 (del4) nucleotides at the end of double strand break generated by SC_GS (corresponding to the lost of the 3′ overhang), other correspond to any other mutagenic NHEJ events detected.

FIG. 6: Effect of Trex2 expression on the nature of deletions induced by different engineered meganucleases.

Size of deletion events were analyzed and the frequency of indicated deletion among all deletion events were calculated after treatment with meganucleases SC_RAG1 (SEQ ID NO: 58 encoded by plasmid pCLS2222, SEQ ID NO: 156), SC_XPC4 (SEQ ID NO: 190 encoded by pCLS2510, SEQ ID NO: 157) and SC_CAPNS1 (SEQ ID NO: 192 encoded by pCLS6163, SEQ ID NO: 158) only (grey histogram) or with Trex2 (SEQ ID NO: 194 encoded by pCLS7673, SEQ ID NO: 154) (black histogram).

FIG. 7: plasmid for SC_GS and SC_GS and Trex2 fusion expression

All fusion constructs were cloned in pCLS1853 (SEQ ID NO: 175), driving their expression by a CMV promoter.

FIG. 8: SSA activity of SC_GS and SC_GS-fused to Trex2.

CHO-K1 cells were co-transfected with the plasmid measuring SSA activity containing the GS_CHO1.1 target and an increasing amounts of SC_GS (pCLS2690, SEQ ID NO: 153), SC_GS-5-Trex2 (pCLS8082, SEQ ID NO: 186), SC_GS-10-Trex2 (pCLS8052, SEQ ID NO: 187), Trex2-5-SC_GS (pCLS8053, SEQ ID NO: 188) or Trex2-10-SC_GS (pCLS8054, SEQ ID NO: 153). Beta-galactosidase activity was detected 72 h after transfection using ONPG and 420 nm optical density detection. The entire process was performed on an automated Velocity 11 BioCel platform.

FIG. 9: Effect of SC_GS fused to Trex2 on mutagenic DSB repair

A: Percentage of GFP+ cells induced on NHEJ model 3 or 4 days after transfection with increasing dose of either SC_GS (pCLS2690, SEQ ID NO: 153), SC_GS-5-Trex2 (pCLS8082, SEQ ID NO: 186), SC_GS-10-Trex2 (pCLS8052, SEQ ID NO: 187), Trex2-5-SC_GS (pCLS8053, SEQ ID NO: 188) or Trex2-10-SC_GS (pCLS8054, SEQ ID NO: 189).

B: Deep-sequencing analysis of deletion events induced by 1 or 6 μg of SC_GS (pCLS2690, SEQ ID NO: 153) or Trex2-10-SC_GS (pCLS8054, SEQ ID NO: 189). C: Percentage of deletion events corresponding to a deletion of 2 (del2), 3 (del3) or 4 (del4) nucleotides at the end of double strand break generated by 1 or 6 μg of SC_GS (pCLS2690, SEQ ID NO: 153) or Trex2-10-SC_GS (pCLS8054, SEQ ID NO: 189), other correspond to any other deletions events detected.

FIG. 10: Effect of Trex-SC_CAPNS1 (SEQ ID NO: 197) fusion on targeted mutagenesis in 293H cell line

Panel A: Percentage of Targeted Mutagenesis [TM] obtained in 293H cell line transfected with SC_CAPNS1 (SEQ ID NO: 192) or Trex-SC_CAPNS1 (SEQ ID NO: 197).

Panel B: Nature of Targeted Mutagenesis obtained in 293H cell line transfected with SC_CAPNS1 (SEQ ID NO: 192) or Trex-SC_CAPNS1 (SEQ ID NO: 197). Del2, Del3 and Del4 correspond to 2, 3 and 4 base pairs deletion events at the cleavage site of CAPNS1. “Other” represents all other TM events.

FIG. 11: Effect of Trex-SC_CAPNS1 (SEQ ID NO: 197) fusion on targeted mutagenesis in 29311 cell line

Panel A: Percentage of Targeted Mutagenesis obtained in Detroit551 cell line transfected with SC_CAPNS1 (SEQ ID NO: 192) or Trex-SC_CAPNS1 (SEQ ID NO: 197).

Panel B: Nature of Targeted Mutagenesis obtained in Detroit551 cell line transfected with SC_CAPNS1 (SEQ ID NO: 192) or Trex-SC_CAPNS1 (SEQ ID NO: 197). Del2, Del3 and Del4 correspond to 2, 3 and 4 base pairs deletion events at the cleavage site of CAPNS1. “Other” represents all other TM events.

FIG. 12: Effect of Tdt expression on targeted mutagenesis in cell line monitoring NHEJ.

Panel A: Percentage of GFP+ cells induced on NHEJ model after co-transfection of 1 μg or 3 μg of SC_GS expressing plasmid (SEQ ID NO: 153) and with either an increasing amount of Tdt expression vector (SEQ ID NO: 153) or with 2 μg of Tdt expressing plasmid (SEQ ID NO: 153), respectively.

Panel B: Percentage of targeted mutagenesis detected by deep sequencing in the vicinity of the GS_CHO1 DNA target present on the NHEJ model, induced by either SC_GS with empty vector or with 2 μg of Tdt encoding vector.

Panel C: Percentage of insertion events within targeted mutagenesis events after co-transfection of the NHEJ model by 3 μg of SC_GS expressing vector with 2 μg of an empty vector or with 2 μg of Tdt encoding plasmid.

Panel D: Percentage of insertion events in function of their size in presence (TDT) or absence (empty) of Tdt.

FIG. 13: Effect of Tdt expression on targeted mutagenesis induced by SC_RAG1 (SEQ ID NO: 58) at endogenous RAG1 locus

Panel A: Percentage of targeted mutagenesis detected by deep sequencing in the vicinity of the SC_RAG1 target induced by co-transfection of 3 μg of SC_RAG1 encoding vector (SEQ ID NO: 156) with different amount of Tdt encoding vector (SEQ ID NO: 202) in 5 μg of total DNA (left part) or in 10 μg of total DNA (right part).

Panel B: Percentage of insertion events within targeted mutagenesis events after co-transfection of 3 μg of SC_RAG1 encoding vector (SEQ ID NO: 156) with different amount of Tdt encoding vector (SEQ ID NO: 202) in 5 μg of total DNA (left part) or in 10 μg of total DNA (right part).

Panel C: Percentage of insertion events in function of their size at endogenous RAG1 locus after co-transfection of 3 μg of SC_RAG1 encoding vector (SEQ ID NO: 156) with different amounts of Tdt encoding vector (SEQ ID NO: 202) in 5 μg of total DNA (left part) or in 10 μg of total DNA (right part).

FIG. 14: Effect of Tdt expression on targeted mutagenesis induced by SC_CAPNS1 (SEQ ID NO: 192) at endogenous CAPNS1 locus

Panel A: Percentage of targeted mutagenesis detected by deep sequencing in the vicinity of the SC_CAPNS1 target induced by co-transfection of 1 μg of SC_CAPNS1 expressing vector (SEQ ID NO: 158) with 2 μg of Tdt encoding plasmid (SEQ ID NO: 202).

Panel B: Percentage of insertion events within targeted mutagenesis events after co-transfection of 3 μg of SC_CAPNS1 expressing vector (SEQ ID NO: 158) with 2 μg of Tdt encoding plasmid (SEQ ID NO: 202).

Panel C: Percentage of insertion events in function of their size at CAPNS1 locus after co-transfection of 3 μg of SC_CAPNS1 expressing vector (SEQ ID NO: 158) with 2 μg of Tdt encoding plasmid (SEQ ID NO: 202).

### DETAILED DESCRIPTION OF THE INVENTION

Unless specifically defined herein below, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

According to a first aspect of the present invention is a method for increasing double-strand break induced mutagenesis at a genomic locus of interest in a cell comprising the steps of:
(i) identifying at said genomic locus of interest at least one DNA target sequence cleavable by one rare-cutting endonuclease;
(ii) engineering said at least one rare-cutting endonuclease in order to generate a loss of genetic information around said DNA target sequence within the genomic locus of interest;
(iii) contacting said DNA target sequence with said at least one rare-cutting endonuclease to generate said loss of genetic information around said DNA target sequence within the genomic locus of interest;
   thereby obtaining a cell in which double-strand break induced mutagenesis at said genomic locus of interest is increased.

In a preferred embodiment, said rare-cutting endonuclease is able to generate one DNA double-strand break in the genomic locus of interest and a loss of genetic information by another enzymatic activity. In a more preferred embodiment, said another enzymatic activity is a nuclease activity. In another more preferred embodiment, said another enzymatic activity is an exonuclease activity. In this preferred embodiment, said rare-cutting endonuclease is a chimeric rare-cutting endonuclease which generates one DNA double-strand break leading to DNA ends, thus processed by an exonuclease activity, allowing the loss of genetic information and preventing any scarless re-ligation of said genomic locus of interest.

In another preferred embodiment, said rare-cutting endonuclease is a chimeric rare-cutting endonuclease which generates one DNA double-strand break leading to DNA ends, thus processed by an enzymatic activity (as illustrated in FIG. 4E) other than a nuclease activity such as polymerase activity (TdT . . . ), a dephosphatase activity, as non-limiting examples.

In a preferred embodiment, said rare-cutting endonuclease of the present invention is a chimeric rare-cutting endonuclease comprising a catalytic domain given in Table 2 (SEQ ID NO: 38-57) and Table 3 (SEQ ID NO: 96-152), a functional mutant, a variant or a derivative thereof. In another preferred embodiment, said chimeric rare-cutting endonuclease of the present invention comprises a catalytic domain selected from the group consisting of Trex (SEQ ID NO: 145-149), and Tdt (SEQ ID NO: 201), functional mutants, variants or derivatives thereof.

In another preferred embodiment, said chimeric rare-cutting endonuclease comprises a catalytic domain of SEQ ID NO: 194, a functional mutant, a variant or a derivative thereof. In another preferred embodiment, said chimeric rare-cutting endonuclease is fused to a protein of SEQ ID NO: 194, a functional mutant, a variant or a derivative thereof. In another preferred embodiment, said chimeric rare-cutting endonuclease is a fusion protein comprising a single chain meganuclease and a protein of SEQ ID NO: 194, a functional mutant, a variant or a derivative thereof. In another preferred embodiment, said chimeric rare-cutting endonuclease is selected from the group consisting of SEQ ID NO: 171-174 and SEQ ID NO: 197.

In another preferred embodiment, said chimeric rare-cutting endonuclease comprises a catalytic domain of SEQ ID NO: 201, a functional mutant, a variant or a derivative thereof. In another preferred embodiment, said chimeric rare-cutting endonuclease is fused to a protein of SEQ ID NO: 201, a functional mutant, a variant or a derivative thereof. In another preferred embodiment, said chimeric rare-cutting endonuclease is a fusion protein comprising a single chain meganuclease and a protein of SEQ ID NO: 201, a functional mutant, a variant or a derivative thereof.

In another aspect the present invention also relates to engineered enzymes and more particularly to chimeric rare-cutting endonucleases able to target a DNA sequence within a genomic locus of interest in order to generate at least one DNA double-strand break and a loss of genetic information by another enzymatic activity around said DNA sequence, thus preventing any scarless re-ligation of said genomic locus of interest by NHEJ. For instance, as a non limiting example, said chimeric rare-cutting endonuclease of the present invention is a fusion protein between a rare-cutting endonuclease which generates one DNA double-strand break at a targeted sequence within the genomic locus of interest, leading to DNA ends and an nuclease domain that is able to process said DNA ends in order to generate a loss of information at the genomic locus of interest. Said nuclease domain can be a exonuclease domain. As another non limiting example, said chimeric rare-cutting endonuclease of the present invention is a fusion protein between a rare-cutting endonuclease which generates one DNA double-strand break at a targeted sequence within the genomic locus of interest, leading to DNA ends and a polymerase activity, such as a template independent polymerase (TdT, . . . ) that is able to process said DNA ends and generate a loss of genetic information at the genomic locus of interest by adding at least one DNA fragment and preventing any scarless re-ligation.

In a preferred embodiment, said rare-cutting endonuclease of the present invention is a chimeric rare-cutting endonuclease comprising a catalytic domain given in Table 2 and Table 3, a functional mutant, a variant or a derivative thereof. In another preferred embodiment, said chimeric rare-cutting endonuclease of the present invention comprises a catalytic domain selected from the group consisting of Trex (SEQ ID NO: 145-149), and Tdt (SEQ ID NO: 201), functional mutants, variants or derivatives thereof.

In another preferred embodiment, said chimeric rare-cutting endonuclease comprises a catalytic domain of SEQ ID NO: 194, a functional mutant, a variant or a derivative thereof. In another preferred embodiment, said chimeric rare-cutting endonuclease is fused to a protein of SEQ ID NO: 194, a functional mutant, a variant or a derivative thereof. In another preferred embodiment, said chimeric rare-cutting endonuclease is a fusion protein comprising a single chain meganuclease and a protein of SEQ ID NO: 194, a functional mutant, a variant or a derivative thereof. In another preferred embodiment, said chimeric rare-cutting endonuclease is selected from the group consisting of SEQ ID NO: 171-174 and SEQ ID NO: 197.

In another preferred embodiment, said chimeric rare-cutting endonuclease comprises a catalytic domain of SEQ ID NO: 201, a functional mutant, a variant or a derivative thereof. In another preferred embodiment, said chimeric rare-cutting endonuclease is fused to a protein of SEQ ID NO: 201, a functional mutant, a variant or a derivative thereof. In another preferred embodiment, said chimeric rare-cutting endonuclease is a fusion protein comprising a single chain meganuclease and a protein of SEQ ID NO: 201, a functional mutant, a variant or a derivative thereof.

In a third aspect, the present invention concerns a method for the generation of at least two-nearby DNA double-strand breaks at a genomic locus of interest to prevent any scarless re-ligation of said genomic locus of interest by NHEJ. In other words, said method comprises the generation of two nearby DNA double-strand breaks into said genomic locus of interest by the introduction of at least one double-strand break creating agent able to generate at least two nearby double-strand breaks such that said at least two nearby DNA double-strand breaks allow the removal of an intervening sequence, as a non limiting example, to prevent any scarless re-ligation of said genomic locus of interest (as illustrated in FIG. 4A to 4C).

According to this third aspect, the present invention concerns a method comprising the steps of:
(i) identifying at said genomic locus of interest one DNA target sequence cleavable by one rare-cutting endonuclease;
(ii) engineering said at least one rare-cutting endonuclease such that said rare-cutting endonuclease is able to generate at least two nearby DNA double-strand breaks in the genomic locus of interest;
(iii) contacting said DNA target sequence with said at least one rare-cutting endonuclease;
   thereby obtaining a cell in which double-strand break induced mutagenesis at said genomic locus of interest is increased.

In a preferred embodiment of this third aspect, said rare-cutting endonuclease of the method is engineered to provide one chimeric rare-cutting endonuclease that is able to generate two nearby DNA double-strand breaks in the genomic locus of interest (as illustrated in FIGS. 4B and 4C). In another preferred embodiment of this second aspect, said rare-cutting endonuclease of the method is engineered to provide one chimeric rare-cutting endonuclease that is able to generate more than two nearby DNA double-strand breaks in the genomic locus of interest; in this preferred embodiment, said one chimeric rare-cutting endonuclease is able to generate three nearby DNA double-strand breaks in the genomic locus of interest.

In a preferred embodiment, said rare-cutting endonuclease of the present invention is a chimeric rare-cutting endonuclease comprising a catalytic domain given in Table 2 and Table 3, a functional mutant, a variant or a derivative thereof. In another preferred embodiment, said chimeric rare-cutting endonuclease of the present invention comprises a catalytic domain selected from the group consisting of Colicin-E7 (SEQ ID NO: 97), I-TevI (SEQ ID NO: 106 or SEQ ID NO: 60; SEQ ID NO: 107-108), NucA (SEQ ID NO: 41 and 112), NucM (SEQ ID NO: 43 and 113), SNase (SEQ ID NO: 45-47 and 116-118), BspD6I (SEQ ID NO: 124-125) a functional mutant, variant or derivative thereof.

In another preferred embodiment, said chimeric rare-cutting endonuclease comprises a catalytic domain of SEQ ID NO: 84, a functional mutant, a variant or a derivative thereof. In another preferred embodiment, said chimeric rare-cutting endonuclease is fused to a protein of SEQ ID NO: 84, a functional mutant, a variant or a derivative thereof. In another preferred embodiment, said chimeric rare-cutting endonuclease is fused to a protein of SEQ ID NO: 54, a functional mutant, a variant or a derivative thereof. In another preferred embodiment, said chimeric rare-cutting endonuclease is a fusion protein comprising a meganuclease and a protein of SEQ ID NO: 54, a functional mutant, a variant or a derivative thereof. In another preferred embodiment, said chimeric rare-cutting endonuclease is selected from the group consisting of SEQ ID NO: 85-87 and SEQ ID NO: 91-93.

In another preferred embodiment, said chimeric rare-cutting endonuclease comprises a catalytic domain selected from the group consisting of SEQ ID NO: 56 and 57, a functional mutant, a variant or a derivative thereof. In another preferred embodiment, said chimeric rare-cutting endonuclease comprises a catalytic domain of SEQ ID NO: 56, a functional mutant, a variant or a derivative thereof. In another preferred embodiment, said chimeric rare-cutting endonuclease comprises a catalytic domain of SEQ ID NO: 57, a functional mutant, a variant or a derivative thereof. In another preferred embodiment, said chimeric rare-cutting endonuclease is fused to a protein of SEQ ID NO: 56, a functional mutant, a variant or a derivative thereof. In another preferred embodiment, said chimeric rare-cutting endonuclease is a fusion protein comprising a meganuclease and a protein of SEQ ID NO: 56, a functional mutant, a variant or a derivative thereof. In another preferred embodiment, said chimeric rare-cutting endonuclease is fused to a protein of SEQ ID NO: 57, a functional mutant, a variant or a derivative thereof. In another preferred embodiment, said chimeric rare-cutting endonuclease is a fusion protein comprising a meganuclease and a protein of SEQ ID NO: 57, a functional mutant, a variant or a derivative thereof. In another preferred embodiment, said chimeric rare-cutting endonuclease is selected from the group consisting of SEQ ID NO: 61-66 and SEQ ID NO: 70-75.

In another embodiment of this third aspect, the present invention implies two engineered rare-cutting endonucleases and comprises the steps of:
(i) identifying at said genomic locus of interest two nearby DNA target sequences respectively cleavable by one rare-cutting endonuclease;
(ii) engineering a first rare-cutting endonuclease able to generate a first DNA double-strand break in the genomic locus of interest;
(iii) engineering a second rare-cutting endonuclease able to generate a second DNA double-strand break in the genomic locus of interest;
(iv) contacting said DNA target sequence with said two rare-cutting endonucleases;
   thereby obtaining a cell in which double-strand break induced mutagenesis at said genomic locus of interest is increased.

In a preferred embodiment, said two engineered rare-cutting endonucleases which respectively target a DNA sequence at a genomic locus of interest are not chimeric rare-cutting endonucleases (as illustrated in FIG. 4A). In another preferred embodiment, said two engineered rare-cutting endonucleases which respectively target a DNA sequence at a genomic locus of interest are chimeric rare-cutting endonucleases. In another preferred embodiment, only one of said two engineered rare-cutting endonucleases, which respectively target a DNA sequence at a genomic locus of interest, is a chimeric rare-cutting endonuclease.

In a preferred embodiment, said at least two nearby DNA double-strand breaks induced into said genomic locus of interest are distant at least 12 bp. In another preferred embodiment, said at least two nearby DNA double-strand break-induced into said genomic locus of interest are distant at least 20 bp, 50 bp, 100, 200, 500 or 1000 bp. In another preferred embodiment, the distance between said at least two nearby DNA double-strand breaks induced into said genomic locus of interest is between 12 bp and 1000 bp, more preferably between 12 bp and 500 bp, more preferably between 12 bp and 200 bp.

In a fourth aspect, the present invention relates to engineered rare-cutting endonucleases and more particularly to chimeric rare-cutting endonucleases, able to target a DNA sequence within a genomic locus of interest in order to generate at said locus of interest at least two-nearby DNA double-strand breaks leading to at least the removal of a DNA fragment and thus preventing any scarless re-ligation of said genomic locus of interest by NHEJ (as illustrated in FIGS. 4A, 4C and 4E). In a preferred embodiment, said chimeric rare-cutting endonucleases comprise at least two catalytic domains. In a more preferred embodiment, said chimeric rare-cutting endonucleases comprise two nuclease domains. In other words, the present invention relates to a chimeric rare-cutting endonuclease to generate at least two nearby DNA double-strand breaks into a genomic locus of interest comprising:
i) a rare-cutting endonuclease;
ii) a peptidic linker;
iii) a nuclease catalytic domain.

In a preferred embodiment, said rare-cutting endonuclease part of said chimeric rare-cutting endonuclease is a meganuclease; in another preferred embodiment, said rare-cutting endonuclease part of said chimeric rare-cutting endonuclease is a I-CreI derived meganuclease. In another preferred embodiment, said rare-cutting endonuclease part of said chimeric rare-cutting endonuclease is a single chain meganuclease derived from I-CreI meganuclease.

In a more preferred embodiment said chimeric rare-cutting endonuclease is a fusion protein between a meganuclease and at least one nuclease catalytic domain. In said more preferred embodiment, said nuclease catalytic domain has an endonuclease activity; alternatively, said nuclease catalytic domain has an exonuclease activity.

In a preferred embodiment, said rare-cutting endonuclease of the present invention is a chimeric rare-cutting endonuclease comprising a catalytic domain given in Table 2 and Table 3, a functional mutant, a variant or a derivative thereof. In another preferred embodiment, said chimeric rare-cutting endonuclease of the present invention comprises a catalytic domain selected from the group consisting of Trex (SEQ ID NO: 145-149), Colicin E7 (SEQ ID NO: 97), 1-TevI (SEQ ID NO: 106 or SEQ ID NO: 60; SEQ ID NO: 107-108), NucA (SEQ ID NO: 41 and 112), NucM (SEQ ID NO: 43 and 113), SNase (SEQ ID NO: 45-47 and 116-118), BspD6I (SEQ ID NO: 124-125), a functional mutant, a variant or a derivative thereof.

In another preferred embodiment, said chimeric rare-cutting endonuclease is a fusion protein comprising a meganuclease and a protein of SEQ ID NO: 145-149, SEQ ID NO: 97, SEQ ID NO: 106 or SEQ ID NO: 60, SEQ ID NO: 107-108, SEQ ID NO: 41 and 112, SEQ ID NO: 43 and 113, SEQ ID NO: 45-47 and 116-118, SEQ ID NO: 124-125, a functional mutant, a variant or a derivative thereof.

In another preferred embodiment, said chimeric rare-cutting endonuclease comprises a catalytic domain of SEQ ID NO: 194, a functional mutant, a variant or a derivative thereof. In another preferred embodiment, said chimeric rare-cutting endonuclease is fused to a protein of SEQ ID NO: 194, a functional mutant, a variant or a derivative thereof. In another preferred embodiment, said rare-cutting endonuclease is a fusion protein comprising a single-chain meganuclease and a protein of SEQ ID NO: 194. In another preferred embodiment, said chimeric rare-cutting endonuclease is selected from the group consisting of SEQ ID NO: 171-174 and SEQ ID NO: 197.

In another preferred embodiment, said chimeric rare-cutting endonuclease comprises a catalytic domain of SEQ ID NO: 84, a functional mutant, a variant or a derivative thereof. In another preferred embodiment, said chimeric rare-cutting endonuclease is fused to a protein of SEQ ID NO: 84, a functional mutant, a variant or a derivative thereof. In another preferred embodiment, said chimeric rare-cutting endonuclease is fused to a protein of SEQ ID NO: 54, a functional mutant, a variant or a derivative thereof. In another preferred embodiment, said chimeric rare-cutting endonuclease is selected from the group consisting of SEQ ID NO: 85-87 and SEQ ID NO: 91-93.

In another preferred embodiment, said chimeric rare-cutting endonuclease comprises a catalytic domain selected from the group consisting of SEQ ID NO: 56 and 57, functional mutants, variants or derivatives thereof. In another preferred embodiment, said chimeric rare-cutting endonuclease comprises a catalytic domain of SEQ ID NO: 56, a functional mutant, a variant or a derivative thereof. In another preferred embodiment, said chimeric rare-cutting endonuclease comprises a catalytic domain of SEQ ID NO: 57, a functional mutant, a variant or a derivative thereof. In another preferred embodiment, said chimeric rare-cutting endonuclease is fused to a protein of SEQ ID NO: 56, a functional mutant, a variant or a derivative thereof. In another preferred embodiment, said chimeric rare-cutting endonuclease is fused to a protein of SEQ ID NO: 57, a functional mutant, a variant or a derivative thereof. In another preferred embodiment, said chimeric rare-cutting endonuclease is selected from the group consisting of SEQ ID NO: 61-66 and SEQ ID NO: 70-75.

In another preferred embodiment, said chimeric rare-cutting endonuclease further comprises a second peptidic linker and a supplementary catalytic domain. In other words, the present invention relates to a chimeric rare-cutting endonuclease able to generate at least two nearby DNA double-strand breaks into a genomic locus of interest comprising:
i) a rare-cutting endonuclease;
ii) a peptidic linker;
iii) a nuclease catalytic domain.
iv) a second peptidic linker
v) a supplementary catalytic domain.

In a preferred embodiment, said supplementary catalytic domain is a nuclease domain; in this case, said chimeric rare-cutting endonuclease is a fusion protein between a rare-cutting endonuclease and two nuclease catalytic domains. In a more preferred embodiment, said chimeric rare-cutting endonuclease is a fusion protein between a meganuclease and two nuclease catalytic domains. In another more preferred embodiment, said chimeric rare-cutting endonuclease is a fusion protein between a meganuclease, one nuclease catalytic domain and one other catalytic domain.

Also encompassed within the scope of the present invention is a chimeric rare-cutting endonuclease able to generate two-nearby double-strand breaks and composed of the DNA-binding domain of a rare-cutting endonuclease and two other nuclease catalytic domains.

In a fifth aspect, the present invention describes a method to identify at a genomic locus of interest a DNA target sequence cleavable at least twice by a fusion protein leading at least to a loss of genetic information and preventing any scarless re-ligation of said genomic locus of interest by NHEJ. More particularly, in this aspect is a method for increasing double-strand break induced mutagenesis at a genomic locus of interest in a cell comprising the steps of:
(i) identifying at said genomic locus of interest one DNA target sequence cleavable by one rare-cutting endonuclease nearby one DNA target sequence cleavable by one frequent-cutting endonuclease;
(ii) engineering said rare-cutting endonuclease such that said rare-cutting endonuclease is able to generate one DNA double-strand break in the genomic locus of interest;
(iii) making a fusion protein between said rare-cutting endonuclease and said frequent-cutting endonuclease;
(iv) contacting said DNA target sequences with said fusion protein to generate at least two nearby double-strand breaks;
   thereby obtaining a cell in which double-strand break induced mutagenesis at said genomic locus of interest is increased.

In a sixth aspect, the present invention relates to fusion proteins able to generate at least two nearby DNA double-strand breaks into a genomic locus of interest comprising one DNA target sequence cleavable by one rare-cutting endonuclease nearby one DNA target sequence cleavable by one frequent-cutting endonuclease. In other words, the present invention relates to a fusion protein comprising:
i) a rare-cutting endonuclease;
ii) a peptidic linker;
ii) a frequent-cutting endonuclease.

In a preferred embodiment, said rare-cutting endonuclease part of said fusion protein is a meganuclease; in another preferred embodiment, said rare-cutting endonuclease part of said fusion protein is a I-CreI derived meganuclease. In another preferred embodiment, said rare-cutting endonuclease part of said fusion protein is a single chain meganuclease derived from I-CreI meganuclease.

In another preferred embodiment, said further fusion protein comprises a second peptidic linker and a supplementary catalytic domain. In other words, the present invention relates to a fusion protein able to generate at least two nearby DNA double-strand breaks into a genomic locus of interest comprising one DNA target sequence cleavable by one rare-cutting endonuclease nearby one DNA target sequence cleavable by one frequent-cutting endonuclease, said fusion protein comprising:
i) a rare-cutting endonuclease;
ii) a peptidic linker;
ii) a frequent-cutting endonuclease;
iv) a second peptidic linker;
v) a supplementary catalytic domain.

In a preferred embodiment, said supplementary catalytic domain is a nuclease domain (as illustrated in FIG. 4D). In another preferred embodiment, said supplementary catalytic domain is a non-nuclease catalytic domain.

The present invention also relates to polynucleotides encoding the endonuclease proteins of the invention, specific vectors (polynucleotidic or not) encoding and/or vectorizing them, compositions and/or kits comprising them, all of them being used or part of a whole to implement methods of the present invention for increasing double-strand break-induced mutagenesis at a genomic locus of interest in a cell. Such kits may contain instructions for use in increasing double-strand break-induced mutagenesis in a cell, packaging materials, one or more containers for the ingredients, and other components used for increasing double-strand break-induced mutagenesis

### DEFINITIONS

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Altered/enhanced/increased/improved cleavage activity, refers to an increase in the detected level of meganuclease cleavage activity, see below, against a target DNA sequence by a second meganuclease in comparison to the activity of a first meganuclease against the target DNA sequence. Normally the second meganuclease is a variant of the first and comprise one or more substituted amino acid residues in comparison to the first meganuclease.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “meganuclease”, is intended an endonuclease having a double-stranded DNA target sequence of 12 to 45 bp. Said meganuclease is either a dimeric enzyme, wherein each domain is on a monomer or a monomeric enzyme comprising the two domains on a single polypeptide.
by “meganuclease domain” is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” it is intended a meganuclease obtained by replacement of at least one residue in the amino acid sequence of the parent meganuclease with a different amino acid. Variants include those with substitutions of 1, 2, 3, 4, 5, 6, 7, 8, 9, 10, 11, 12, 13, 14, 15, 16, 17, 18, 19, 20 or more amino acid residues. Such variants may have 75, 80, 85, 90, 95, 97.5, 98, 99, 99.5% or more homology or identity (or any intermediate value within this range) to a base or parental meganuclease sequence.
by “peptide linker”, “peptidic linker” or “peptide spacer” it is intended to mean a peptide sequence which allows the connection of different monomers in a fusion protein and the adoption of the correct conformation for said fusion protein activity and which does not alter the specificity of either of the monomers for their targets. Peptide linkers can be of various sizes, from 3 amino acids to 50 amino acids as a non limiting indicative range. Non-limiting examples of such peptidic linkers are given in Table 1.
by “related to”, particularly in the expression “one cell type related to the chosen cell type or organism”, is intended a cell type or an organism sharing characteristics with said chosen cell type or said chosen organism; this cell type or organism related to the chosen cell type or organism, can be derived from said chosen cell type or organism or not.
by “subdomain” it is intended the region of a LAGLIDADG homing endonuclease core domain which interacts with a distinct part of a homing endonuclease DNA target half-site.
by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the targeted gene comprised in the locus of interest and the repair matrix, wherein the genomic sequence containing the DNA target is replaced by the third portion of the repair matrix and a variable part of the first and second portions of the repair matrix.
by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.
by “screening” it is intended to mean the sequential or simultaneous selection of one or more meganuclease variant (s) which exhibits a specified phenotype such as altered cleavage activity.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “I-CreI” is intended the wild-type I-CreI having the sequence of pdb accession code 1 g9y, corresponding to the sequence SEQ ID NO: 1 in the sequence listing.
by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present Patent Application all the I-CreI variants described comprise an additional Alanine after the first Methionine of the wild type I-CreI sequence as shown in SEQ ID NO: 195. These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present Patent Application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.
by “I-CreI site” is intended a 22 to 24 bp double-stranded DNA sequence which is cleaved by I-CreI. I-CreI sites include the wild-type non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 2), also called C1221.
by “domain” or “core domain” is intended the “LAGLIDADG homing endonuclease core domain” which is the characteristic αββαββα a fold of the homing endonucleases of the LAGLIDADG family, corresponding to a sequence of about one hundred amino acid residues. Said domain comprises four beta-strands (β₁β₂β₃β₄) folded in an anti-parallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94.
by “subdomain” is intended the region of a LAGLIDADG homing endonuclease core domain which interacts with a distinct part of a homing endonuclease DNA target half-site.
by “chimeric DNA target” or “hybrid DNA target” it is intended the fusion of a different half of two parent meganuclease target sequences. In addition at least one half of said target may comprise the combination of nucleotides which are bound by at least two separate subdomains (combined DNA target). Is also encompassed in this definition a DNA target sequence, comprising a rare-cutting endonuclease target sequence (20-24 bp) and a frequent-cutting endonuclease target sequence (4-8 bp), recognized by a chimeric rare-cutting endonuclease according to the present invention.
by “beta-hairpin” is intended two consecutive beta-strands of the antiparallel beta-sheet of a LAGLIDADG homing endonuclease core domain (β₁β₂ or β₃β₄) which are connected by a loop or a turn,
by “single-chain meganuclease”, “single-chain chimeric meganuclease”, “single-chain meganuclease derivative”, “single-chain chimeric meganuclease derivative” or “single-chain derivative” is intended a meganuclease comprising two LAGLIDADG homing endonuclease domains or core domains linked by a peptidic spacer as described in WO2009095793. The single-chain meganuclease is able to cleave a chimeric DNA target sequence comprising one different half of each parent meganuclease target sequence.
by “DNA target”, “DNA target sequence”, “target sequence”, “target-site”, “target”, “site”, “site of interest”, “recognition site”, “polynucleotide recognition site”, “recognition sequence”, “homing recognition site”, “homing site”, “cleavage site” is intended a 20 to 24 bp double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. Said DNA target sequence is qualified of “cleavable” by an endonuclease, when recognized within a genomic sequence and known to correspond to the DNA target sequence of a given endonuclease or a variant of such endonuclease. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicate above for C1221. Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of different halves of two parent meganuclease target sequences. In addition at least one half of said target may comprise the combination of nucleotides which are bound by at least two separate subdomains (combined DNA target).
The term “endonuclease” refers to any wild-type or variant enzyme capable of catalyzing the hydrolysis (cleavage) of bonds between nucleic acids within of a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site of about 12-45 base pairs (bp) in length, more preferably of 14-45 bp. Rare-cutting endonucleases significantly increase HR by inducing DNA double-strand breaks (DSBs) at a defined locus (Rouet, Smih et al. 1994; Rouet, Smih et al. 1994; Choulika, Perrin et al. 1995; Pingoud and Silva 2007). Rare-cutting endonucleases can for example be a homing endonuclease (Paques and Duchateau 2007), a chimeric Zinc-Finger nuclease (ZFN) resulting from the fusion of engineered zinc-finger domains with the catalytic domain of a restriction enzyme such as FokI (Porteus and Carroll 2005) or a chemical endonuclease (Eisenschmidt, Lanio et al. 2005; Arimondo, Thomas et al. 2006; Simon, Cannata et al. 2008). In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences (Kalish and Glazer 2005). Such chemical endonucleases are comprised in the term “endonuclease” according to the present invention. Rare-cutting endonucleases can also be for example TALENs, a new class of chimeric nucleases using a Fokl catalytic domain and a DNA binding domain derived from Transcription Activator Like Effector (TALE), a family of proteins used in the infection process by plant pathogens of the Xanthomonas genus (Boch, Scholze et al. 2009; Moscou and Bogdanove 2009; Christian, Cermak et al. 2010; Li, Huang et al. 2010). The functional layout of a FokI-based TALE-nuclease (TALEN) is essentially that of a ZFN, with the Zinc-finger DNA binding domain being replaced by the TALE domain. As such, DNA cleavage by a TALEN requires two DNA recognition regions flanking an unspecific central region. Rare-cutting endonucleases encompassed in the present invention can also be derived from TALENs.

Rare-cutting endonuclease can be a homing endonuclease, also known under the name of meganuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. An expression such as “double-strand break creating agent” can be used to qualify a rare-cutting endonuclease according to the present invention.

In the wild, meganucleases are essentially represented by homing endonucleases. Homing Endonucleases (HEs) are a widespread family of natural meganucleases including hundreds of proteins families (Chevalier and Stoddard 2001). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffolds to derive novel, highly specific endonucleases.

HEs belong to four major families. The LAGLIDADG family, named after a conserved peptidic motif involved in the catalytic center, is the most widespread and the best characterized group. Seven structures are now available. Whereas most proteins from this family are monomeric and display two LAGLIDADG motifs, a few have only one motif, and thus dimerize to cleave palindromic or pseudo-palindromic target sequences.

Although the LAGLIDADG peptide is the only conserved region among members of the family, these proteins share a very similar architecture. The catalytic core is flanked by two DNA-binding domains with a perfect two-fold symmetry for homodimers such as I-CreI (Chevalier, Monnat et al. 2001), I-MsoI (Chevalier, Turmel et al. 2003) and I-CeuI (Spiegel, Chevalier et al. 2006) and with a pseudo symmetry for monomers such as I-SceI (Moure, Gimble et al. 2003), I-DmoI (Silva, Dalgaard et al. 1999) or I-AniI (Bolduc, Spiegel et al. 2003). Both monomers and both domains (for monomeric proteins) contribute to the catalytic core, organized around divalent cations. Just above the catalytic core, the two LAGLIDADG peptides also play an essential role in the dimerization interface. DNA binding depends on two typical saddle-shaped αββαββα folds, sitting on the DNA major groove. Other domains can be found, for example in inteins such as PI-PfuI (Ichiyanagi, Ishino et al. 2000) and PI-SceI (Moure, Gimble et al. 2002), whose protein splicing domain is also involved in DNA binding.

The making of functional chimeric meganucleases, by fusing the N-terminal I-DmoI domain with an I-CreI monomer (Chevalier, Kortemme et al. 2002; Epinat, Arnould et al. 2003); International PCT Application WO 03/078619 (Cellectis) and WO 2004/031346 (Fred Hutchinson Cancer Research Center, Stoddard et al)) have demonstrated the plasticity of LAGLIDADG proteins.

Different groups have also used a semi-rational approach to locally alter the specificity of the I-CreI (Seligman, Stephens et al. 1997; Sussman, Chadsey et al. 2004); International PCT Applications WO 2006/097784, WO 2006/097853, WO 2007/060495 and WO 2007/049156 (Cellectis); (Arnould, Chames et al. 2006; Rosen, Morrison et al. 2006; Smith, Grizot et al. 2006), I-SceI (Doyon, Pattanayak et al. 2006), PI-SceI (Gimble, Moure et al. 2003) and I-MsoI (Ashworth, Havranek et al. 2006).

In addition, hundreds of I-CreI derivatives with locally altered specificity were engineered by combining the semi-rational approach and High Throughput Screening:
Residues Q44, R68 and R70 or Q44, R68, D75 and 177 of I-CreI were mutagenized and a collection of variants with altered specificity at positions±3 to 5 of the DNA target (5NNN DNA target) were identified by screening (International PCT Applications WO 2006/097784 and WO 2006/097853 (Cellectis); (Arnould, Chames et al. 2006; Smith, Grizot et al. 2006).
Residues K28, N30 and Q38 or N30, Y33 and Q38 or K28, Y33, Q38 and S40 of I-CreI were mutagenized and a collection of variants with altered specificity at positions ±8 to 10 of the DNA target (10NNN DNA target) were identified by screening (Arnould, Chames et al. 2006; Smith, Grizot et al. 2006); International PCT Applications WO 2007/060495 and WO 2007/049156 (Cellectis)).

Two different variants were combined and assembled in a functional heterodimeric endonuclease able to cleave a chimeric target resulting from the fusion of two different halves of each variant DNA target sequence ((Arnould, Chames et al. 2006; Smith, Grizot et al. 2006); International PCT Applications WO 2006/097854 and WO 2007/034262).

Furthermore, residues 28 to 40 and 44 to 77 of I-CreI were shown to form two partially separable functional subdomains, able to bind distinct parts of a homing endonuclease target half-site (Smith, Grizot et al. 2006); International PCT Applications WO 2007/049095 and WO 2007/057781 (Cellectis)).

The combination of mutations from the two subdomains of I-CreI within the same monomer allowed the design of novel chimeric molecules (homodimers) able to cleave a palindromic combined DNA target sequence comprising the nucleotides at positions ±3 to 5 and ±8 to 10 which are bound by each subdomain ((Smith, Grizot et al. 2006); International PCT Applications WO 2007/049095 and WO 2007/057781 (Cellectis)).

The method for producing meganuclease variants and the assays based on cleavage-induced recombination in mammal or yeast cells, which are used for screening variants with altered specificity are described in the International PCT Application WO 2004/067736; (Epinat, Arnould et al. 2003; Chames, Epinat et al. 2005; Arnould, Chames et al. 2006). These assays result in a functional LacZ reporter gene which can be monitored by standard methods.

The combination of the two former steps allows a larger combinatorial approach, involving four different subdomains. The different subdomains can be modified separately and combined to obtain an entirely redesigned meganuclease variant (heterodimer or single-chain molecule) with chosen specificity. In a first step, couples of novel meganucleases are combined in new molecules (“half-meganucleases”) cleaving palindromic targets derived from the target one wants to cleave. Then, the combination of such “half-meganucleases” can result in a heterodimeric species cleaving the target of interest. The assembly of four sets of mutations into heterodimeric endonucleases cleaving a model target sequence or a sequence from different genes has been described in the following Cellectis International patent applications: XPC gene (WO2007/093918), RAG gene (WO2008/010093), HPRT gene (WO2008/059382), beta-2 microglobulin gene (WO2008/102274), Rosa26 gene (WO2008/152523), Human hemoglobin beta gene (WO2009/13622) and Human interleukin-2 receptor gamma chain gene (WO2009019614).

These variants can be used to cleave genuine chromosomal sequences and have paved the way for novel perspectives in several fields, including gene therapy.

Examples of such endonuclease include I-Sce I, I-Chu I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, PI-Tsp I, I-MsoI.

A homing endonuclease can be a LAGLIDADG endonuclease such as I-SceI, I-CreI, I-CeuI, I-MsoI, and I-DmoI.

Said LAGLIDADG endonuclease can be I-Sce I, a member of the family that contains two LAGLIDADG motifs and functions as a monomer, its molecular mass being approximately twice the mass of other family members like I-CreI which contains only one LAGLIDADG motif and functions as homodimers.

Endonucleases mentioned in the present application encompass both wild-type (naturally-occurring) and variant endonucleases. Endonucleases according to the invention can be a “variant” endonuclease, i.e. an endonuclease that does not naturally exist in nature and that is obtained by genetic engineering or by random mutagenesis, i.e. an engineered endonuclease. This variant endonuclease can for example be obtained by substitution of at least one residue in the amino acid sequence of a wild-type, naturally-occurring, endonuclease with a different amino acid. Said substitution(s) can for example be introduced by site-directed mutagenesis and/or by random mutagenesis. In the frame of the present invention, such variant endonucleases remain functional, i.e. they retain the capacity of recognizing and specifically cleaving a target sequence to initiate gene targeting process.

The variant endonuclease according to the invention cleaves a target sequence that is different from the target sequence of the corresponding wild-type endonuclease. Methods for obtaining such variant endonucleases with novel specificities are well-known in the art.

Endonucleases variants may be homodimers (meganuclease comprising two identical monomers) or heterodimers (meganuclease comprising two non-identical monomers).

Endonucleases with novel specificities can be used in the method according to the present invention for gene targeting and thereby integrating a transgene of interest into a genome at a predetermined location.
by “parent meganuclease” it is intended to mean a wild type meganuclease or a variant of such a wild type meganuclease with identical properties or alternatively a meganuclease with some altered characteristic in comparison to a wild type version of the same meganuclease. In the present invention the parent meganuclease can refer to the initial meganuclease from which the first series of variants are derived in step (a) or the meganuclease from which the second series of variants are derived in step (b), or the meganuclease from which the third series of variants are derived in step (k).
By “delivery vector” or “delivery vectors” is intended any delivery vector which can be used in the present invention to put into cell contact or deliver inside cells or subcellular compartments agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” is also intended delivery methods to perform transfection
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells.
By “integrative lentiviral vectors (or LV)”, is meant such vectors as non limiting example, that are able to integrate the genome of a target cell.
At the opposite by “non integrative lentiviral vectors (or NILV)” is meant efficient gene delivery vectors that do not integrate the genome of a target cell through the action of the virus integrase.

One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors. A vector according to the present invention comprises, but is not limited to, a YAC (yeast artificial chromosome), a BAC (bacterial artificial), a baculovirus vector, a phage, a phagemid, a cosmid, a viral vector, a plasmid, a RNA vector or a linear or circular DNA or RNA molecule which may consist of chromosomal, non chromosomal, semi-synthetic or synthetic DNA. In general, expression vectors of utility in recombinant DNA techniques are often in the form of “plasmids” which refer generally to circular double stranded DNA loops which, in their vector form are not bound to the chromosome. Large numbers of suitable vectors are known to those of skill in the art. Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracyclin, rifampicin or ampicillin resistance in E. coli. Preferably said vectors are expression vectors, wherein a sequence encoding a polypeptide of interest is placed under control of appropriate transcriptional and translational control elements to permit production or synthesis of said polypeptide. Therefore, said polynucleotide is comprised in an expression cassette. More particularly, the vector comprises a replication origin, a promoter operatively linked to said encoding polynucleotide, a ribosome binding site, a RNA-splicing site (when genomic DNA is used), a polyadenylation site and a transcription termination site. It also can comprise an enhancer or silencer elements. Selection of the promoter will depend upon the cell in which the polypeptide is expressed. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-□-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.
Inducible promoters may be induced by pathogens or stress, more preferably by stress like cold, heat, UV light, or high ionic concentrations (reviewed in Potenza C et al. 2004, In vitro Cell Dev Biol 40:1-22). Inducible promoter may be induced by chemicals (reviewed in (Moore, Samalova et al. 2006); (Padidam 2003); (Wang, Zhou et al. 2003); (Zuo and Chua 2000).

Delivery vectors and vectors can be associated or combined with any cellular permeabilization techniques such as sonoporation or electroporation or derivatives of these techniques.
By cell or cells is intended any prokaryotic or eukaryotic living cells, cell lines derived from these organisms for in vitro cultures, primary cells from animal or plant origin.
By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines. These cells thus represent a more valuable model to the in vivo state they refer to.
In the frame of the present invention, “eukaryotic cells” refer to a fungal, plant or animal cell or a cell line derived from the organisms listed below and established for in vitro culture. More preferably, the fungus is of the genus Aspergillus, Penicillium, Acremonium, Trichoderma, Chrysoporium, Mortierella, Kluyveromyces or Pichia; More preferably, the fungus is of the species Aspergillus niger, Aspergillus nidulans, Aspergillus oryzae, Aspergillus terreus, Penicillium chrysogenum, Penicillium citrinum, Acremonium Chrysogenum, Trichoderma reesei, Mortierella alpine, Chrysosporium lucknowense, Kluyveromyces lactis, Pichia pastoris or Pichia ciferrii.

More preferably the plant is of the genus Arabidospis, Nicotiana, Solanum, lactuca, Brassica, Oryza, Asparagus, Pisum, Medicago, Zea, Hordeum, Secale, Triticum, Capsicum, Cucumis, Cucurbita, Citrullis, Citrus, Sorghum; More preferably, the plant is of the species Arabidospis thaliana, Nicotiana tabaccum, Solanum lycopersicum, Solanum tuberosum, Solanum melongena, Solanum esculentum, Lactuca saliva, Brassica napus, Brassica oleracea, Brassica rapa, Oryza glaberrima, Oryza sativa, Asparagus officinalis, Pisum sativum, Medicago sativa, zea mays, Hordeum vulgare, Secale cereal, Triticum aestivum, Triticum durum, Capsicum sativus, Cucurbita pepo, Citrullus lanatus, Cucumis melo, Citrus aurantifolia, Citrus maxima, Citrus medica, Citrus reticulata.

More preferably the animal cell is of the genus Homo, Rattus, Mus, Sus, Bos, Danio, Canis, Felis, Equus, Salmo, Oncorhynchus, Gallus, Meleagris, Drosophila, Caenorhabditis; more preferably, the animal cell is of the species Homo sapiens, Rattus norvegicus, Mus musculus, Sus scrofa, Bos taurus, Danio rerio, Canis lupus, Felis catus, Equus caballus, Salmo salar, Oncorhynchus mykiss, Gallus gallus, Meleagris gallopavo, Drosophila melanogaster, Caenorhabditis elegans.
by “homologous” is intended a sequence with enough identity to another one to lead to homologous recombination between sequences, more particularly having at least 95% identity, preferably 97% identity and more preferably 99%.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.
by “mutation” is intended the substitution, deletion, insertion of one, two, three, four, five, six, seven, eight, nine, ten, eleven, twelve, thirteen, fourteen, fifteen, sixteen, seventeen, eighteen, nineteen, twenty or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.
In the frame of the present invention, the expression “double-strand break-induced mutagenesis” (DSB-induced mutagenesis) refers to a mutagenesis event consecutive to an NHEJ event following an endonuclease-induced DSB, leading to insertion/deletion at the cleavage site of an endonuclease.
By “gene” is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns, a 3′ untranslated region. The gene may further comprise a terminator, enhancers and/or silencers.
As used herein, the term “transgene” refers to a sequence encoding a polypeptide. Preferably, the polypeptide encoded by the transgene is either not expressed, or expressed but not biologically active, in the cell, tissue or individual in which the transgene is inserted. Most preferably, the transgene encodes a therapeutic polypeptide useful for the treatment of an individual.
The term “gene of interest” or “GOI” refers to any nucleotide sequence encoding a known or putative gene product.
As used herein, the term “locus” is the specific physical location of a DNA sequence (e.g. of a gene) on a chromosome. The term “locus” usually refers to the specific physical location of an endonuclease's target sequence on a chromosome. Such a locus, which comprises a target sequence that is recognized and cleaved by an endonuclease according to the invention, is referred to as “locus according to the invention”. Also, the expression “genomic locus of interest” is used to qualify a nucleic acid sequence in a genome that can be a putative target for a double-strand break according to the invention. By “endogenous genomic locus of interest” is intended a native nucleic acid sequence in a genome, i.e., a sequence or allelic variations of this sequence that is naturally present at this genomic locus. It is understood that the considered genomic locus of interest of the present invention can be between two overlapping genes the considered endonuclease's target sequences are located in two different genes. It is understood that the considered genomic locus of interest of the present invention can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria or chloroplasts as non-limiting examples.
By the expression “loss of genetic information” is understood the elimination or addition of at least one given DNA fragment (at least one nucleotide) or sequence, bordering the recognition sites of the endonucleases of the present invention and leading to a change of the original sequence around said endonuclease-cutting sites, within the genomic locus of interest. This loss of genetic information can be, as a non-limiting example, the elimination of an intervening sequence between two endonuclease-cutting sites; it can also be, in another non-limiting example, the result of an exonuclease DNA-ends processing activity after a unique endonuclease DNA double-strand break. In this last case, loss of genetic information within the genomic locus of interest is generated “around said DNA target sequence”, i.e. around the endonuclease-cutting site (DSB), taken as reference. It can also be, in other non-limiting examples, the result of DNA-ends processing activities by other enzymes, after a unique endonuclease DNA double-strand break, such as polymerase activity (TdT . . . ), dephosphatase activity . . . .
By the expression “two nearby DNA double strand breaks” within the genomic locus of interest, is meant two endonucleases cutting sites distant at between 12 bp and 1000 bp.
By “scarless re-ligation” is intended the perfect re-ligation event, without loss of genetic information (no insertion/deletion events) of the DNA broken ends through NHEJ process after the creation of a double-strand break event. The present invention relates to a method to increase double-strand break mediated mutagenesis by avoiding any such “scarless re-ligation” process.
By “fusion protein” is intended the result of a well-known process in the art consisting in the joining of two or more genes which originally encode for separate proteins, the translation of said “fusion gene” resulting in a single polypeptide with functional properties derived from each of the original proteins.
By “chimeric rare-cutting endonuclease” is meant any fusion protein comprising a rare-cutting endonuclease. Said rare-cutting endonuclease might be at the N-terminus part of said chimeric rare-cutting endonuclease; at the opposite, said rare-cutting endonuclease might be at the C-terminus part of said chimeric rare-cutting endonuclease. A “chimeric rare-cutting endonuclease” according to the present invention which comprises two catalytic domains can be described as “bi-functional” or as “bi-functional meganuclease”. A “chimeric rare-cutting endonuclease” according to the present invention which comprises more than two catalytic domains can be described as “multi-functional” or as “multi-functional meganuclease”. As non-limiting examples, chimeric rare-cutting endonucleases according to the present invention can be a fusion protein between a rare-cutting endonuclease and one catalytic domain; chimeric rare-cutting endonucleases according to the present invention can also be a fusion protein between a rare-cutting endonuclease and two catalytic domains. As mentioned previously, the rare-cutting endonuclease part of chimeric rare-cutting endonucleases according to the present invention can be a meganuclease comprising either two identical monomers, either two non identical monomers, or a single chain meganuclease. The rare-cutting endonuclease part of chimeric rare-cutting endonucleases according to the present invention can also be the DNA-binding domain of a rare-cutting endonuclease. In other non-limiting examples, chimeric rare-cutting endonucleases according to the present invention can be derived from a TALE-nuclease (TALEN), i.e., a fusion between a DNA-binding domain derived from a Transcription Activator Like Effector (TALE) and one or two catalytic domains.
By “frequent-cutting endonuclease” is intended an endonuclease typically having a polynucleotide recognition site of about 4-8 base pairs (bp) in length, more preferably of 4-6 bp.
By a “TALE-nuclease” (TALEN) is intended a fusion protein consisting of a DNA-binding domain derived from a Transcription Activator Like Effector (TALE) and one FokI catalytic domain, that need to dimerize to form an active entity able to cleave a DNA target sequence.
By “catalytic domain” is intended the protein domain or module of an enzyme containing the active site of said enzyme; by active site is intended the part of said enzyme at which catalysis of the substrate occurs. Enzymes, but also their catalytic domains, are classified and named according to the reaction they catalyze. The Enzyme Commission number (EC number) is a numerical classification scheme for enzymes, based on the chemical reactions they catalyze (http://www.chem.qmul.ac.uk/iubmb/enzyme/). In the scope of the present invention, any catalytic domain can be fused to a rare-cutting endonuclease to generate a chimeric rare-cutting endonuclease. Non-limiting examples of such catalytic domains are given in table 2 and in table 3 with a GenBank or NCBI or UniProtKB/Swiss-Prot number as a reference.
By “nuclease catalytic domain” is intended the protein domain comprising the active site of an endonuclease or an exonuclease enzyme. Non-limiting examples of such catalytic domains are given in table 2 and in table 3 with a GenBank or NCBI or UniProtKB/Swiss-Prot number as a reference.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

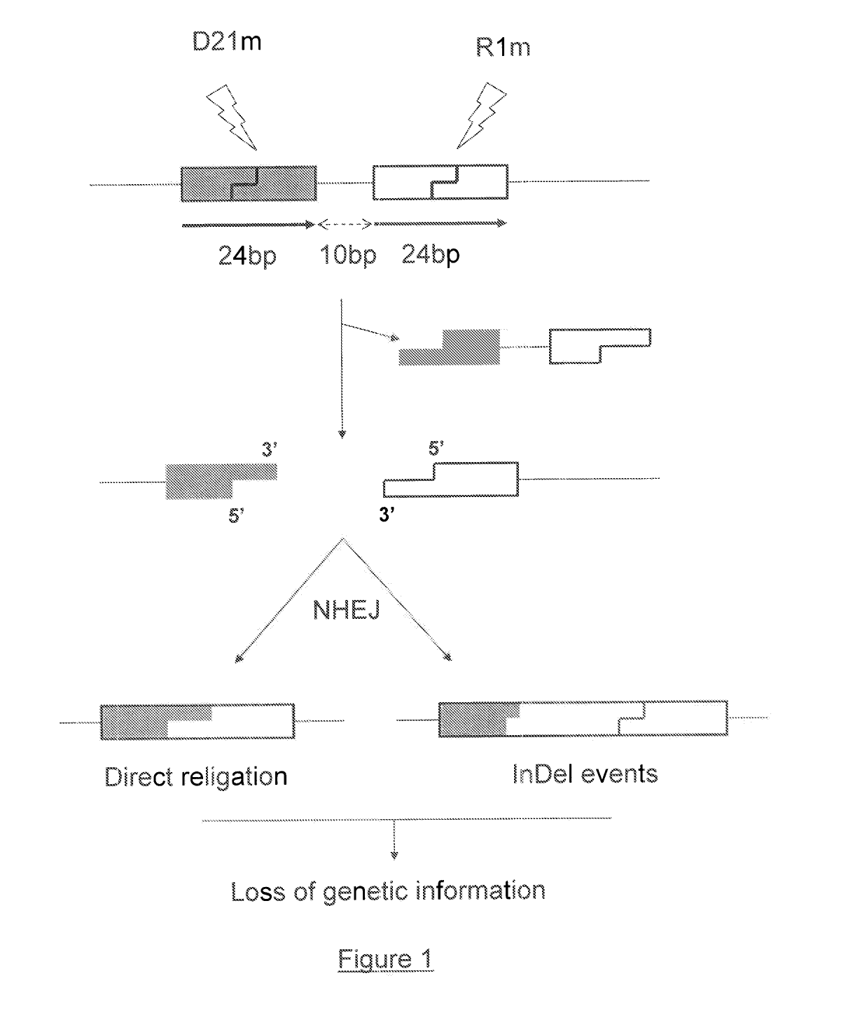

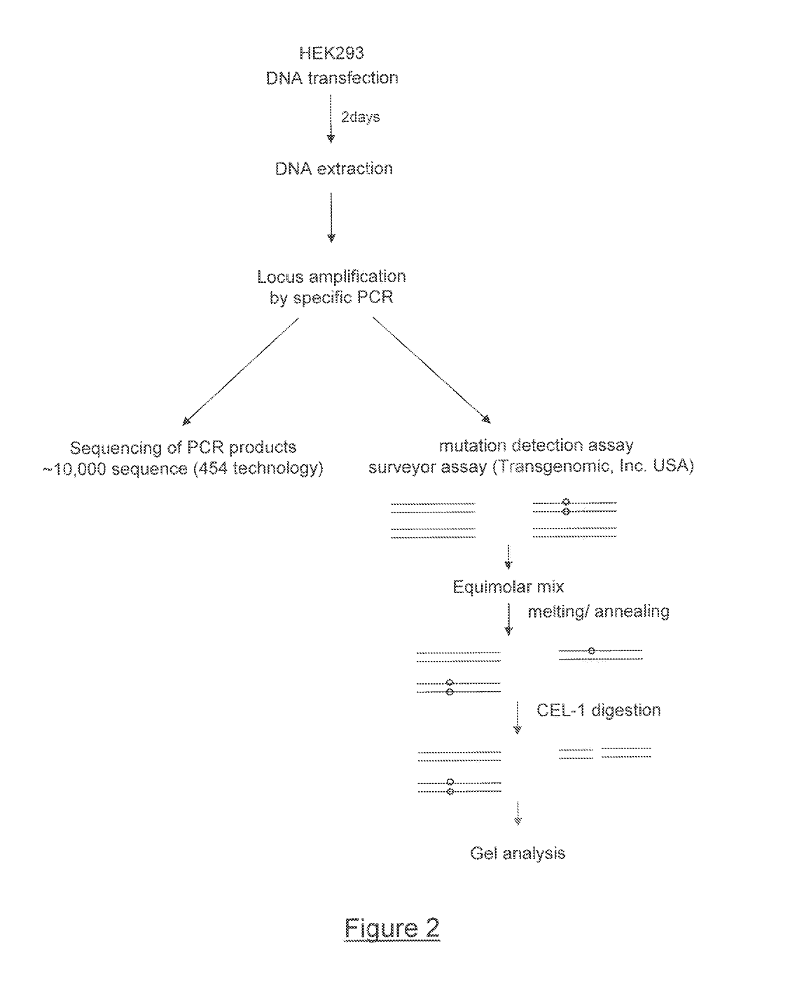

Two engineered single-chain meganucleases called R1 or R1m (SEQ ID NO: 58) and D21 or D21m (SEQ ID NO: 59) are produced using the methods disclosed in International PCT Applications WO2003078619, WO2004/067736, WO2006/097784, WO2006/097853, WO2007/060495, WO 2007/049156, WO 2006/097854, WO2007/034262, WO 2007/049095, WO2007/057781 and WO2009095793 (Cellectis) and in (Chames, Epinat et al. 2005; Arnould, Chames et al. 2006; Smith, Grizot et al. 2006). These meganucleases, derived from I-CreI, are designed to recognize two different DNA sequences, neither of which are recognized by wild-type I-CreI. (recognition sequences, respectively, tgttctcaggtacctcagccag SEQ ID NO: 3 and aaacctcaagtaccaaatgtaa SEQ ID NO: 4). Expression of these two meganucleases is driven by a CMV promoter and a polyA signal sequence. The two corresponding recognition sites are cloned in close proximity to generate the target plasmid. For this example, the recognition sites are separated by 10 bp (FIG. 1). DNA cleavage by a meganuclease generates characteristic 4-nt 3′-OH overhangs. The simultaneous cleavage of both sites is expected to eliminate the intervening sequence and therefore abolish “scarless” re-ligation by NHEJ (FIG. 1).

Human HEK293 cells are transiently co-transfected with two plasmids carrying the expression cassette for R1 (SEQ ID NO: 58) and D21 (SEQ ID NO: 59), as well as the target plasmid. For comparison, HEK293 cells are transiently co-transfected with the target plasmid and only one meganuclease-expressing plasmid. DNA is extracted 2 days post-transfection and targeted mutagenesis is assessed by a mutation detection assay as depicted in FIG. 2 (surveyor assay from Transgenomic, Inc. USA). High-fidelity PCR amplification of the DNA encompassing the two recognition sites is performed using appropriate specific primers. The same PCR amplification is performed on genomic DNA extracted from cells transfected with the target plasmid alone. After quantification and purification, equimolar amounts of PCR products are mixed in an annealing buffer and a fraction of this mixture is subjected to a melting/annealing step, resulting in the formation of distorted duplex DNA through random re-annealing of mutant and wild-type DNA. CEL-1 enzyme (surveyor assay from Transgenomic, Inc. USA) is added to specifically cleave the DNA duplexes at the sites of mismatches. The CEL-1 cleaved samples are resolved on analytical gel, stained with ethidium bromide and the DNA bands are quantified using densitometry. The frequency of mutagenesis can then be calculated essentially as described in Miller et al (2007).

PCR products from cells transfected with the target plasmid and (a) an empty plasmid; (b) one meganuclease expressing plasmid or; (c) two plasmids expressing respectively R1 (SEQ ID NO: 58) and D21 (SEQ ID NO: 59), are also analyzed by high-throughput sequencing (FIG. 2). In this case, PCR amplification is performed with appropriate primers to obtain a fragment flanked by specific adaptor sequences (adaptor A: 5′-CCATCTCATCCCTGCGTGTCTCCGAC-NNNN-3′, SEQ ID NO: 5 and adaptor B, CCTATCCCCTGTGTGCCTTGGCAGTCTCAG-3′, SEQ ID NO: 6) provided by the company (GATC Biotech AG, Germany) offering sequencing service on a 454 sequencing system (454 Life Sciences). Approximately 10,000 exploitable sequences are obtained per PCR pool and then analyzed for the presence of site-specific insertion or deletion events. The inventers are able to show that deletion of the intervening sequence or microsequence following the creation of two DNA DSBs greatly enhances the site-specific NHEJ-driven mutation rate. This deletion is observed only when both meganucleases are introduced into the cells.

### Example 2

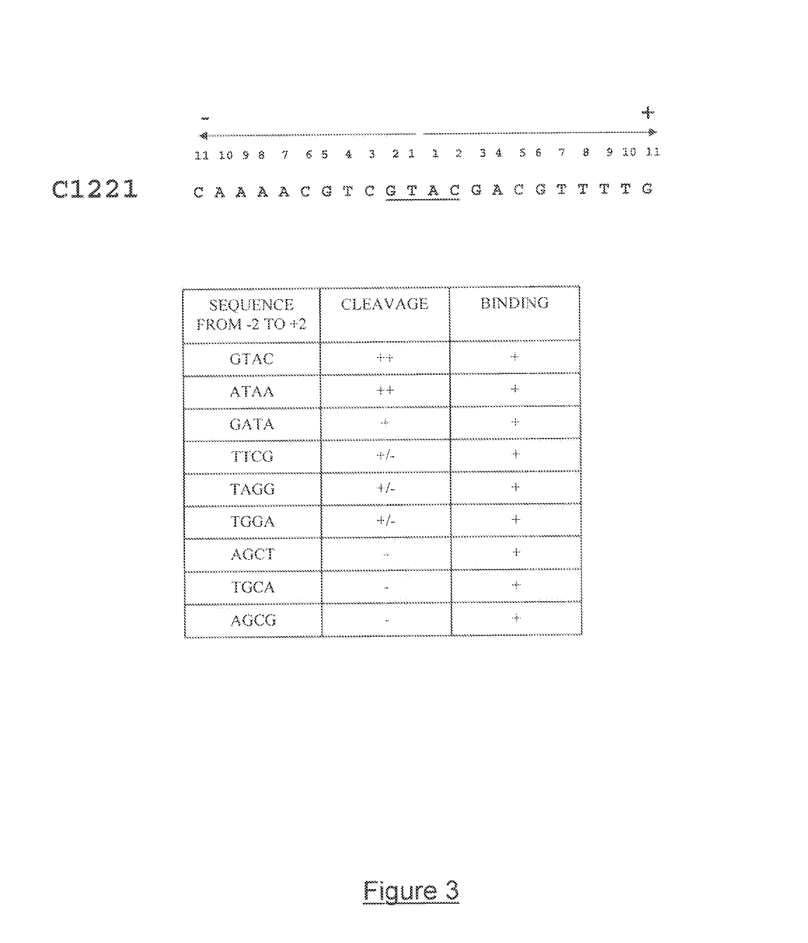

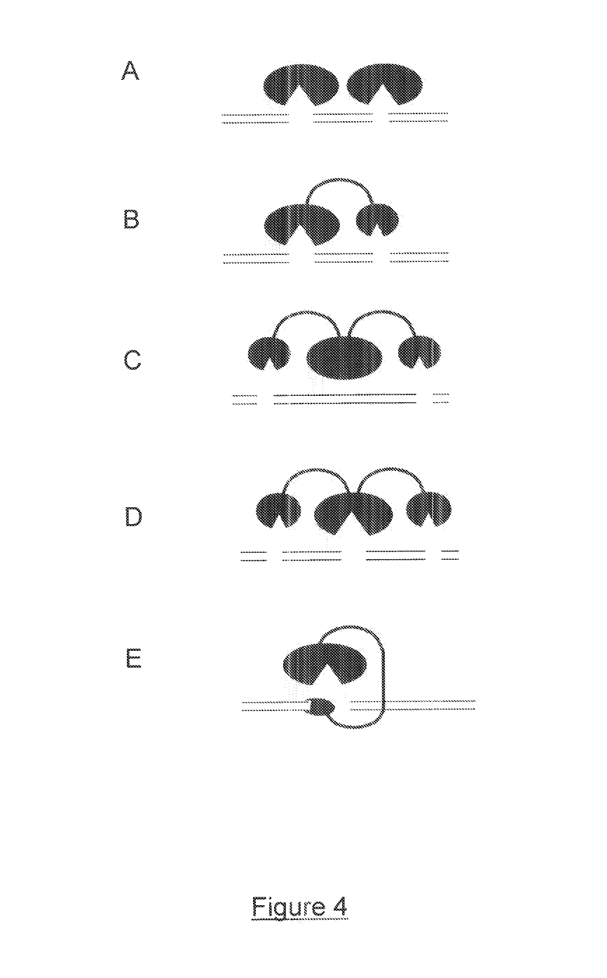

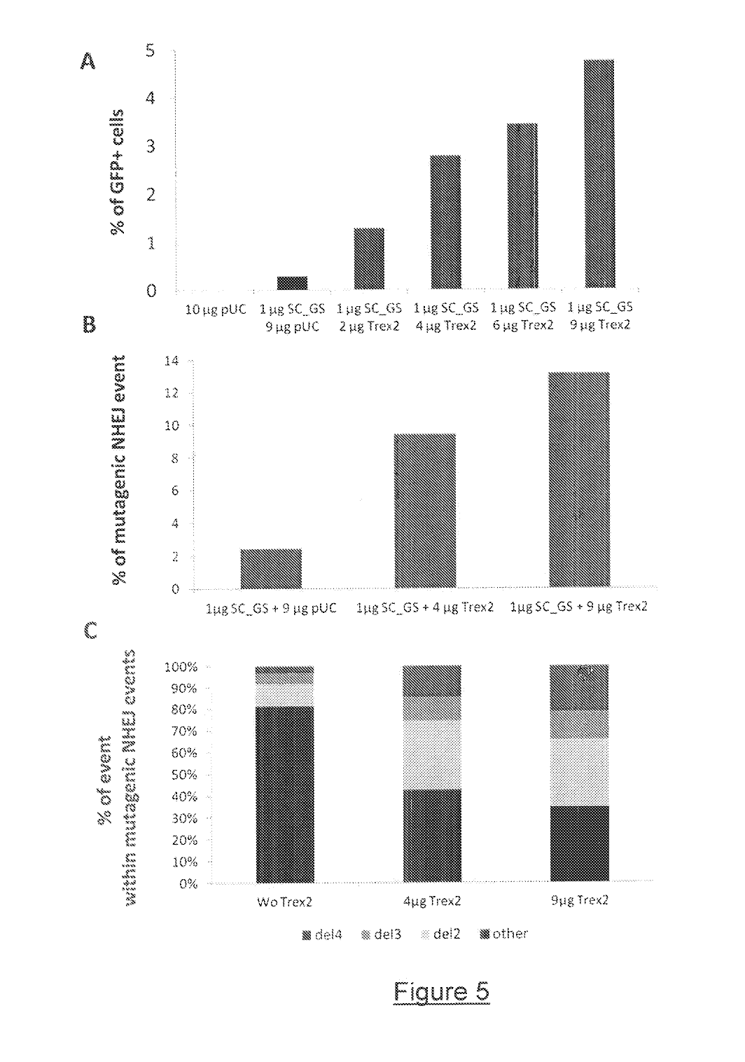

In this example, an engineered single-chain meganuclease derived from I-CreI (described in International PCT Applications WO2003078619, WO 2004/067736, WO 2006/097784, WO 2006/097853, WO 2007/060495, WO 2007/049156, WO 2006/097854, WO 2007/034262, WO 2007/049095, WO 2007/057781, WO 2008/010093 and WO2009095793 (Cellectis) and in (Chames, Epinat et al. 2005; Arnould, Chames et al. 2006; Smith, Grizot et al. 2006)) is fused to various nuclease domains to create a bi-functional meganuclease. To obtain maximal activity, 31 different linkers are tested ranging in size from 3 to 26 amino acids (Table 1). Fusions are made using 18 different catalytic domains (Table 2) that are chosen based on their having essentially non-specific nuclease activity. Altogether, a library of 1116 different constructs are created via fusion to the N- or C-terminus of the engineered single-chain I-CreI-derived meganuclease, generating a collection of potential bi-functional meganucleases. Expression of these chimeric meganucleases are driven by a CMV promoter and a polyA signal sequence. The activity of each chimeric protein is assessed using our yeast assay previously described in International PCT Applications WO 2004/067736 and in (Epinat, Arnould et al. 2003; Chames, Epinat et al. 2005; Arnould, Chames et al. 2006; Smith, Grizot et al. 2006). To monitor DNA cleavage activity resulting from the addition of the new catalytic domain, an I-CreI DNA target sequence is selected that can be bound but not cleaved by the wild-type meganuclease. This target contains 4-nucleotide substitutions at positions—2 to +2 (FIG. 3). Enzymes exhibiting cleavage activity are then tested following protocols described in example 1, except that the target plasmid carried only one cleavage site for the I-CreI meganuclease.

To further validate the high rate of site-specific mutagenesis induced by a bi-functional chimeric endonuclease, the same strategy is applied to an engineered single-chain meganuclease designed to cleave the human RAG1 gene as described in International PCT Applications WO2003078619, WO 2008/010093 and WO2009095793. The bi-functional meganuclease is tested for its ability to induced NHEJ-driven mutation at its endogenous cognate recognition site. The mutagenesis activity is quantified by high-throughput sequencing of PCR products as described in example 1. PCR amplification is performed on genomic DNA extracted from meganuclease-transfected cells using appropriate primers.

The bi-functional meganucleases displayed an increased mutation rate since the intervening sequence is deleted, thereby preventing “scarless” re-ligation of DNA ends through NHEJ.

TABLE 1
sequence of linkers used to fuse the catalytic domains
to meganucleases

| | Amino | | Size | | SEQ |
|---|---|---|---|---|---|
| Name (PDB) | Acids | Length | (Da) | Sequence | ID NO |
| 1a8h_1 | 285-287 | 3 | 6.636 | NVG | 7 |
| 1dnpA_1 | 130-133 | 4 | 7.422 | DSVI | 8 |
| 1d8cA_2 | 260-263 | 4 | 8.782 | IVEA | 9 |
| 1ckqA_3 | 169-172 | 4 | 9.91 | LEGS | 10 |
| 1sbp_1 | 93-96 | 4 | 10.718 | YTST | 11 |
| 1ev7A_1 | 169-173 | 5 | 11.461 | LQENL | 12 |
| 1alo_3 | 360-364 | 5 | 12.051 | VGRQP | 13 |
| 1amf_1 | 81-85 | 5 | 13.501 | LGNSL | 14 |
| 1adjA_3 | 323-328 | 6 | 14.835 | LPEEKG | 15 |
| 1fcdC_1 | 76-81 | 6 | 14.887 | QTYQPA | 16 |
| 1al3_2 | 265-270 | 6 | 15.485 | FSHSTT | 17 |
| 1g3p_1 | 99-105 | 7 | 17.903 | GYTYINP | 18 |
| 1acc_3 | 216-222 | 7 | 19.729 | LTKYKSS | 19 |
| 1ahjB_1 | 106-113 | 8 | 17.435 | SRPSESEG | 20 |
| 1acc_1 | 154-161 | 8 | 18.776 | PELKQKSS | 21 |
| 1af7_1 | 89-96 | 8 | 22.502 | LTTNLTAF | 22 |
| 1heiA_1 | 322-330 | 9 | 13.534 | TATPPGSVT | 23 |
| 1bia_2 | 268-276 | 9 | 16.089 | LDNFINRPV | 24 |
| 1igtB_1 | 111-119 | 9 | 19.737 | VSSAKTTAP | 25 |
| 1nfkA_1 | 239-248 | 10 | 13.228 | DSKAPNASNL | 26 |
| 1au7A_1 | 103-112 | 10 | 20.486 | KRRTTISIAA | 27 |
| 1bpoB_1 | 138-148 | 11 | 21.645 | PVKMFDRHSSL | 28 |
| 1b0pA_2 | 625-635 | 11 | 26.462 | APAETKAEPMT | 29 |
| 1c05A_2 | 135-148 | 14 | 23.819 | YTRLPERSELPAEI | 30 |
| 1gcb_1 | 57-70 | 14 | 27.39 | VSTDSTPVTNQKSS | 31 |
| 1bt3A_1 | 38-51 | 14 | 28.818 | YKLPAVTTMKVRPA | 32 |
| 1b3oB_2 | 222-236 | 15 | 20.054 | IARTDLKKNRDYPLA | 33 |
| 16vpA_6 | 312-332 | 21 | 23.713 | TEEPGAPLTTPPTLHGNQARA | 34 |
| 1dhx_1 | 81-101 | 21 | 42.703 | ARFTLAVGDNRVLDMASTYFD | 35 |
| 1b8aA_1 | 95-120 | 26 | 31.305 | IVVLNRAETPLPLDPTGKVKAELDTR | 36 |
| 1qu6A_1 | 79-106 | 28 | 51.301 | ILNKEKKAVSPLLLTTTNSSEGLSMGNY | 37 |
| NFS1 | — | 20 | — | GSDITKSKISEKMKGQGPSG | 78 |
| NFS2 | — | 23 | — | GSDITKSKISEKMKGLGPDGRKA | 79 |
| CFS1 | — | 10 | — | SLTKSKISGS | 80 |
| RM2 | — | 32 | — | AAGGSALTAGALSLTAGALSLTAGALSGGGGS | 94 |
| BQY | — | 25 | — | AAGASSVSASGHIAPLSLPSSPPSVGS | 95 |
| QGPSG | — | 5 | — | QGPSG | 67 |
| LGPDGRKA | — | 8 | — | LGPDGRKA | 68 |

TABLE 2
sequences of the catalytic domains fused to meganucleases.

| DATABASE | | SEQ ID | |
|---|---|---|---|
| reference | Name | NO | SEQUENCE |
| GenBank: ACC85607.1 | MmeI | 38 | alswneirrkaiefskrwedasdensqakpflidffevfgitn |
| | | | krvatfehavkkfakahkeqsrgfvdlfwpgilliemksrgk |
| | | | dldkaydqaldyfsgiaerdlpryvlvcdfqrfrltdlitkesve |
| | | | fllkdlyqnvrsfgfiagyqtqvikpqd |
| GenBank: EAJ03172.1 | EsaSSII | 39 | aalsfpeirtrlqafakqwkqaerenadaklfwarfyecfgir |
| | | | pesatiyekavdkldgsrgfidsfipgllivehkskgkdlnsaf |
| | | | tqasdyftalaegerpryiivsdfarfrlydlktdtqveckladis |
| | | | khagwfrflvegeatpeivees |
| NCBI Reference | CstMI | 40 | vmapttvfdratirhnltefldrwldrikqweaenrpatessh |
| Sequence: NP_862240.1 | | | dqqfwgdlldcfgvnardlylyqrsakrastgrtgkidmfm |
| | | | pgkvigeakslgvplddayaqaldyllggtianshmpayvv |
| | | | csnfetlrvtrlnrtyvgdsadwditfplaeidehieqlaflady |
| | | | etsayreee |
| GenBank: CAA45962.1 | NucA | 41 | qvppltelspsisvhlllgnpsgatptkltpdnylmvknqyal |
| | | | synnskgtanwvawqlnsswlgnaerqdnfrpdktlpagw |
| | | | vrvtpsmysgsgydrghiapsadrtkttednaatflmtnmm |
| | | | pqtpdnuntwgnledycrelvsqgkelyivagpngslgkp |
| | | | lkgkvtvpkstwkivvvldspgsglegitantrviavnipnd |
| | | | pelnndwraykvsvdelesltgydflsnvspniqtsieskvd |
| | | | n |
| P25736 (END1_ECOLI), | EndA Escherichia | 42 | eginsfsqakaaavkvhadapgtfycgckinwqgkkgvvd |
| UniProtKB/Swiss-Prot | coli | | lqscgyqvrknenrasrvewehvvpawqfghqrqcwqdg |
| | | | grkncakdpvyrkmesdmhnlqpsvgevngdrgnfmys |
| | | | qwnggegqygqcamkvdfkekaaepparargaiartyfy |
| | | | mrdqynltlsrqqtqlfnawnkmypvtdwecerderiakv |
| | | | qgnhnpyvqracqarks |
| P37994 (NUCM_DICD3), | NucM | 43 | aagqdinnftqakaaaakihqdapgtfycgckinwqgkkgt |
| UniProtKB/Swiss-Prot | | | pdlascgyqvrkdanrasriewehvvpawqfghqrqcwq |
| | | | dggrknctkddvyrqietdlhnlqpaigevngdrgnfmysq |
| | | | wnggerqygqcemkidfksqlaepperargaiartyfymr |
| | | | drynlnlsrqqtqlfdawnkqypattwectrekriaavqgnh |
| | | | npyvqqacspdaapyynglslimiaavatvaarwltpaghl |
| | | | psd |
| P0A3S3 (NUCE_STRPN), | EndA Streptococcus | 44 | ikqmpsapnspktnlsqkkgaseapsqalaesvltdavksqi |
| UniProtKB/Swiss-Prot | pneumonia | | kgslewngsgafivngnktnldakvsskpyadnktktvgke |
| | | | typtvanallskatrqyknrketgngstswtppgwhqvknlk |
| | | | gsythavdrghllgyaliggldgfdastsnpkniavqtawan |
| | | | qaqaeystgqnyyeskyrkaldqnkrvryrvtlyyasnedlv |
| | | | psasqieakssdgelefnvlvpnvqkglqldyrtgevtvtq |
| P00644 (NUC_STAAU), | SNase | 45 | atstkklhkepatlikaidgdtvklmykgqpmtfrlllvdtpet |
| UniProtKB/Swiss-Prot | Staphylococcus | | khpkkgvekygpeasaftkkmvenakkievefdkgqrtdk |
| | aureus | | ygrglayiyadgkmvnealvrqglakvayvykpnntheqh |
| | | | lrkseaqakkeklniwsednadsgq |
| P43270 (NUC_STAHY), | SNase | 46 | gpfksaglsnaneqtykvirvidgdtiivdkdgkqqnlrmig |
| UniProtKB/Swiss-Prot | Staphylococcus | | vdtpetvkpntpvqpygkeasdftkrhltnqkvrleydkqek |
| | hyicus | | drygrtlayvwlgkemfneklakeglarakfyrpnykyqeri |
| | | | eqaqkqaqklkkniwsn |
| P29769 (NUC_SHIFL), | SNase Shigella | 47 | wadfrgevvrildgdtidvlvnrqfirvrladidapesgqafgs |
| UniProtKB/Swiss-Prot | flexneri | | rarqrladltfrqevqvtekevdrygrtlgvvyaplqypggqt |
| | | | qltninaimvqegmawayryygkptdaqmyeyekearrq |
| | | | rlglwsdpnagepwkwrrasknatn |
| P94492 (YNCB_BACSU), | Bacillus subtilis | 48 | cgsnhaaknhsdsngteqvsqdthsneynqteqkagtphsk |
| UniProtKB/Swiss-Prot | yncB | | nqkklvnvtldraidgdtikviyngkkdtvryllvdtpetkkp |
| | | | nscvqpygedaskrnkelvnsgklqlefdkgdrrkygrlla |
| | | | yvyydgksvqetllkeglarvayvyepntkyidqfrldeqea |
| | | | ksdklsiwsksgyvtnrgfngcvk |
| P00641 (ENRN_BPT7), | Endodeoxyribonuclease | 49 | agygakgirkvgafrsgledkvskqleskgikfeyeewkvp |
| UniProtKB/Swiss-Prot | 1 Enterobacteria | | yvipasnhtytpdfllpngifvetkglwesddrkkhllireqh |
| | phage T7 | | peldirivfsssrtklykgsptsygefcekhgikfadklipaew |
| | | | ikepkkevpfdrlkrkggkk |
| P38447 (NUCG_BOVIN), | EndoG bovine | 50 | aglpavpgapagggpgelakyglpgvaqlksrasyvlcydp |
| UniProtKB/Swiss-Prot | | | rtrgalwvveqlrpeglrgdgnrsscdfheddsvhayhratn |
| | | | adyrgsgfdrghlaaaanhrwsqkamddtfylsnvapqvp |
| | | | hlnqnawnnlekysrsltrtyqnvyvctgplflprteadgksy |
| | | | vkyqvigknhvavpthffkvlileaaggqielrsyvmpnap |
| | | | vdeaiplehflvpiesierasgllfvpnilaragslkaitagsk |
| Q56239 (MUTS_THET8), | ttSmr DNA | 51 | ggyggvkmegmlkgegpgplppllqqyvelrdrypdylll |
| UniProtKB/Swiss-Prot | mismatch repair | | fqvgdfyecfgedaerlaralglvlthktskdfttpmagipira |
| | protein mutS | | fdayaerllkmgfrlavadqvepaeeaeglvrrevtqlltpgtl |
| | | | t |
| Q53H47(SETMR_HUMAN), | Cleavage domain of | 52 | hlkqigkvkkldkwvpheltenqknrrfevssslilrnhnepf |
| UniProtKB/Swiss-Prot | Metnase | | ldrivtcdekwilydnrrrsaqwldqeeapkhfpkpilhpkk |
| | | | vmvtiwwsaaglihysflnpgetitsekyaqeidemnqklq |
| | | | rlqlalvnrkgpillhdnarphvaqptlqklnelgyevlphpp |
| | | | yspdllptnyhvfkhlnnflqgkrfhnqqdaenafqefvesq |
| | | | stdfyatginqlisrwqkcvdcngsyfd |
| GenBank: AAF19759.1 | Vvn | 53 | appssfsaakqqavkiyqdhpisfycgcdiewqgkkgipnl |
| | | | etcgyqvrkqqtrasriewehvvpawqfghhrqcwqkggr |
| | | | kncskndqqfrlmeadlhnltpaigevngdrsnfnfsqwng |
| | | | vdgvsygrcemqvnfkqrkvmppdrargsiartylymsqe |
| | | | ygfqlskqqqqlmqawnksypvdewectrddriakiqgn |
| | | | hnpfvqqscqtq |
| Q47112 (CEA7_ECOLX), | ColE7 nuclease | 54 | Krnkpgkatgkgkpvnnkwlnnagkdlgspvpdrianklr |
| UniProtKB/Swiss-Prot | domain | | dkefksfddfrkkfweevskdpelskqfsrnnndrmkvgk |
| | | | apktrtqdvsgkrtsfelhhekpisqnggvydmdnisvvtp |
| | | | krhidihrgk |
| P34081 (HMUI_BPSP1), | I-HmuI | 55 | mewkdikgyeghyqvsntgevysiksgktlkhqipkdgy |
| UniProtKB/Swiss-Prot | | | hriglfkggkgktfqvhrlvaihfcegyeeglvvdhkdgnkd |
| | | | nnlstnlrwvtqkinvenqmsrgtlnvskaqqiakiknqkpi |
| | | | ivispdgiekeypstkcaceelgltrgkvtdvlkghrihhkgy |
| | | | tfryklng |
| P13299 (TEV1_BPT4), | I-TevI | 56 | ksgiyqikntlnnkvyvgsakdfekrwkrhfkdlekgchssi |
| UniProtKB/Swiss-Prot | | | klqrsfnkhgnvfecsileeipyekdliierenfwikelnskin |
| | | | gyniadatfgdtcsthplkeeiiklasetvkakmlklgpdgrk |
| | | | alyskpgskngrwnpethkfckcgvriqtsaytcskcrnr |
| Q38419 (TEV3_BPR03), | I-TevIII | 57 | nyrkiwidangpipkdsdgrtdeihhkdgnrenndldnlm |
| UniProtKB/Swiss-Prot | | | clsiqehydihlaqkdyqachaiklrmkyspeeiselaskaa |
| | | | ksreiqifnipevrakniasikskiengtfhlldgeiqrksnlnr |
| | | | valgihnfqqaehiakvk |

TABLE 3
sequences of the catalytic domains fused to meganucleases.

| GENBANK/ | | | |
|---|---|---|---|
| SWISS- | | | |
| PROT | | SEQ | |
| ID | NAME | ID NO | FASTA SEQUENCE |
| ACC85607.1 | MmeI | 96 | >gi\|186469979\|gb\|ACC85607.1\| MmeI [Methylophilus methylotrophus] |
| | | | MALSWNEIRRKAIEFSKRWEDASDENSQAKPFLIDFFEVFGITNKRVATFEHAVKKF |
| | | | AKAHKEQSRGFVDLFWPGILLIEMKSRGKDLDKAYDQALDYFSGIAERDLPRYVLVC |
| | | | DFQRFRLTDLITKESVEFLLKDLYQNVRSFGFIAGYQTQVIKPQDPINIKAAERMGK |
| | | | LHDTLKLVGYEGHALELYLVRLLFCLFAEDTTIFEKSLFQEYIETKTLEDGSDLAHH |
| | | | INTLFYVLNTPEQKRLKNLDEHLAAFPYINGKLFEEPLPPAQFDKAMREALLDLCSL |
| | | | DWSRISPAIFGSLFQSIMDAKKRRNLGAHYTSEANILKLIKPLFLDELWVEFEKVKN |
| | | | NKNKLLAFHKKLRGLTFFDPACGCGNFLVITYRELRLLEIEVLRGLHRGGQQVLDIE |
| | | | HLIQINVDQFFGIEIEEFPAQIAQVALWLTDHQMNMKISDEFGNYFARIPLKSTPHI |
| | | | LNANALQIDWNDVLEAKKCCFILGNPPFVGKSKQTPGQKADLLSVFGNLKSASDLDL |
| | | | VAAWYPKAAHYIQTNANIRCAFVSTNSITQGEQVSLLWPLLLSLGIKINFAHRTFSW |
| | | | TNEASGVAAVHCVIIGFGLKDSDEKIIYEYESINGEPLAIKAKNINPYLRDGVDVIA |
| | | | CKRQQPISKLPSMRYGNKPTDDGNFLFTDEEKNQFITNEPSSEKYFRRFVGGDEFIN |
| | | | NTSRWCLWLDGADISEIRAMPLVLARIKKVQEFRLKSSAKPTRQSASTPMKFFYISQ |
| | | | PDTDYLLIPETSSENRQFIPIGFVDRNVISSNATYHIPSAEPLIFGLLSSTMHNCWM |
| | | | RNVGGRLESRYRYSASLVYNTFPWIQPNEKQSKAIEEAAFAILKARSNYPNESLAGL |
| | | | YDPKTMPSELLKAHQKLDKAVDSVYGFKGPNTEIARIAFLFETYQKMTSLLPPEKEI |
| | | | KKSKGKN |
| Q47112.2 | Colicin-E7 | 97 | >gi\|12644448\|sp\|Q47112.2\|CEA7_ECOLX RecName: |
| | (CEA7_ECOLX) | | Full = Colicin-E7 |
| | | | MSGGDGRGHNSGAHNTGGNINGGPTGLGGNGGASDGSGWSSENNPWGGGSGSGVHWG |
| | | | GGSGHGNGGGNSNSGGGSNSSVAAPMAFGFPALAAPGAGTLGISVSGEALSAAIADI |
| | | | FAALKGPFKFSAWGIALYGILPSEIAKDDPNMMSKIVTSLPAETVTNVQVSTLPLDQ |
| | | | ATVSVTKRVTDVVKDTRQHIAVVAGVPMSVPVVNAKPTRTPGVFHASFPGVPSLTVS |
| | | | TVKGLPVSTTLPRGITEDKGRTAVPAGFTFGGGSHEAVIRFPKESGQKPVYVSVTDV |
| | | | LTPAQVKQRQDEEKRLQQEWNDAHPVEVAERNYEQARAELNQANKDVARNQERQAKA |
| | | | VQVYNSRKSELDAANKTLADAKAEIKQFERFAREPMAAGHRMWQMAGLKAQRAQTDV |
| | | | NNKKAAFDAAAKEKSDADVALSSALERRKQKENKEKDAKAKLDKESKRNKPGKATGK |
| | | | GKPVNNKWLNNAGKDLGSPVPDRIANKLRDKEFKSFDDFRKKFWEEVSKDPELSKQF |
| | | | SRNNNDRMKVGKAPKTRTQDVSGKRTSFELHHEKPISQNGGVYDMDNISVVTPKRHI |
| | | | DIHRGK |
| CAA38134.1 | EndA | 98 | >gi\|47374\|emb\|CAA38134.1\| EndA [Streptococcus pneumoniae] |
| | | | MNKKTRQTLIGLLVLLLLSTGSYYIKQMPSAPNSPKTNLSQKKQASEAPSQALAESV |
| | | | LTDAVKSQIKGSLEWNGSGAFIVNGNKTNLDAKVSSKPYADNKTKTVGKETVPTVAN |
| | | | ALLSKATRQYKNRKETGNGSTSWTPPGWHQVKNLKGSYTHAVDRGHLLGYALIGGLD |
| | | | GFDASTSNPKNIAVQTAWANQAQAEYSTGQNYYESKVRKALDQNKRVRYRVTLYYAS |
| | | | NEDLVPSASQIEAKSSDGELEFNVLVPNVQKGLQLDYRTGEVTVTQ |
| P25736.1 | Endo I | 99 | >gi\|119325\|sp\|P25736.1\|END1_ECOLI RecName: |
| | (END1_ECOLI) | | Full = Endonuclease-1; AltName: Full = Endonuclease I; |
| | | | Short = Endo I; Flags: Precursor |
| | | | MYRYLSIAAVVLSAAFSGPALAEGINSFSQAKAAAVKVHADAPGTFYCGCKINWQGK |
| | | | KGVVDLQSCGYQVRKNENRASRVEWEHVVPAWQFGHQRQCWQDGGRKNCAKDPVYRK |
| | | | MESDMHNLQPSVGEVNGDRGNFMYSQWNGGEGQYGQCAMKVDFKEKAAEPPARARGA |
| | | | IARTYFYMRDQYNLTLSRQQTQLFNAWNKMYPVTDWECERDERIAKVQGNHNPYVQR |
| | | | ACQARKS |
| Q14249.4 | Human Endo | 100 | >gi\|317373579\|sp\|Q14249.4\|NUCG_HUMAN RecName: |
| | G | | Full = Endonuclease G, mitochondrial; Short = Endo G; Flags: |
| | (NUCG_HUMAN) | | Precursor |
| | | | MRALRAGLTLASGAGLGAVVEGWRRRREDARAAPGLLGRLPVLPVAAAAELPPVPGG |
| | | | PRGPGELAKYGLPGLAQLKSRESYVLCYDPRTRGALWVVEQLRPERLRGDGDRRECD |
| | | | FREDDSVHAYHRATNADYRGSGFDRGHLAAAANHRWSQKAMDDTFYLSNVAPQVPHL |
| | | | NQNAWNNLEKYSRSLTRSYQNVYVCTGPLFLPRTEADGKSYVKYQVIGKNHVAVPTH |
| | | | FFKVLILEAAGGQIELRTYVMPNAPVDEAIPLERFLVPIESIERASGLLFVPNILAR |
| | | | AGSLKAITAGSK |
| P38447.1 | Bovine Endo | 101 | >gi\|585596\|sp\|P38447.1\|NUCG_BOVIN RecName: |
| | G | | Full = Endonuclease G, mitochondrial; Short = Endo G; Flags: |
| | (NUCG_BOVIN) | | Precursor |
| | | | MQLLRAGLTLALGAGLGAAAESWWRQRADARATPGLLSRLPVLPVAAAAGLPAVPGA |
| | | | PAGGGPGELAKYGLPGVAQLKSRASYVLCYDPRTRGALWVVEQLRPEGLRGDGNRSS |
| | | | CDFHEDDSVHAYHRATNADYRGSGFDRGHLAAAANHRWSQKAMDDTFYLSNVAPQVP |
| | | | HLNQNAWNNLEKYSRSLTRTYQNVYVCTGPLFLPRTEADGKSYVKYQVIGKNHVAVP |
| | | | THFFKVLILEAAGGQIELRSYVMPNAPVDEAIPLEHFLVPIESIERASGLLFVPNIL |
| | | | ARAGSLKAITAGSK |
| AAW33811.1 | R.HinP1I | 102 | >gi\|57116674\|gb\|AAW33811.1\| R.HinP1I restriction |
| | | | endonuclease [Haemophilus influenzae] |
| | | | MNLVELGSKTAKDGFKNEKDIADRFENWKENSEAQDWLVTMGHNLDEIKSVKAVVLS |
| | | | GYKSDINVQVLVFYKDALDIHNIQVKLVSNKRGFNQIDKHWLAHYQEMWKFDDNLLR |
| | | | ILRHFTGELPPYHSNTKDKRRMFMTEFSQEEQNIVLNWLEKNRVLVLTDILRGRGDF |
| | | | AAEWVLVAQKVSNNARWILRNINEVLQHYGSGDISLSPRGSINFGRVTIQRKGGDNG |
| | | | RETANMLQFKIDPTELFDI |
| AAO93095.1 | I-BasI | 103 | >gi\|29838473\|gb\|AAO93095.1\| I-Basi [Bacillus phage Bastille] |
| | | | MFQEEWKDVTGFEDYYEVSNKGRVASKRTGVIMAQYKINSGYLCIKFTVNKKRTSHL |
| | | | VHRLVAREFCEGYSPELDVNHKDTDRMNNNYDNLEWLTRADNLKDVRERGKLNTHTA |
| | | | REALAKVSKKAVDVYTKDGSEYIATYPSATEAAEALGVQGAKISTVCHGKRQHTGGY |
| | | | HFKFNSSVDPNRSVSKK |
| AAK09365.1 | I-BmoI | 104 | >gi\|12958590\|gb\|AAK09365.1\|AF321518_2 intron encoded I- |
| | | | BmoI [Bacillus mojavensis] |
| | | | MKSGVYKITNKNTGKFYIGSSEDCESRLKVHFRNLKNNRHINRYLNNSFNKHGEQVF |
| | | | IGEVIHILPIEEAIAKEQWYIDNFYEEMYNISKSAYHGGDLTSYHPDKRNIILKRAD |
| | | | SLKKVYLKMTSEEKAKRWQCVQGENNPMFGRKHTETTKLKISNHNKLYYSTHKNPFK |
| | | | GKKHSEESKTKLSEYASQRVGEKNPFYGKTHSDEFKTYMSKKFKGRKPKNSRPVIID |
| | | | GTEYESATEASRQLNVVPATILHRIKSKNEKYSGYFYK |
| P34081.1 | I-HmuI | 105 | >gi\|465641\|sp\|P34081.1\|HMUI_BPSP1 RecName: Full = DNA |
| | | | endonuclease I-HmuI; AltName: Full = HNH homing |
| | | | endonuclease I-HmuI |
| | | | MEWKDIKGYEGHYQVSNTGEVYSIKSGKTLKHQIPKDGYHRIGLFKGGKGKTFQVHR |
| | | | LVAIHFCEGYEEGLVVDHKDGNKDNNLSTNLRWVTQKINVENQMSRGTLNVSKAQQI |
| | | | AKIKNQKPIIVISPDGIEKEYPSTKCACEELGLTRGKVTDVLKGHRIHHKGYTFRYK |
| | | | LNG |
| P13299.2 | I-TevI | 106 | >gi\|6094464\|sp\|P13299.2\|TEV1_BPT4 RecName: Full = Intron- |
| | | | associated endonuclease 1; AltName: Full = I-TevI; AltName: |
| | | | Full = IRF protein |
| | | | MKSGIYQIKNTLNNKVYVGSAKDFEKRWKRHFKDLEKGCHSSIKLQRSFNKHGNVFE |
| | | | CSILEEIPYEKDLIIERENFWIKELNSKINGYNIADATFGDTCSTHPLKEEIIKKRS |
| | | | ETVKAKMLKLGPDGRKALYSKPGSKNGRWNPETHKFCKCGVRIQTSAYTCSKCRNRS |
| | | | GENNSFFNHKHSDITKSKISEKMKGKKPSNIKKISCDGVIFDCAADAARHFKISSGL |
| | | | VTYRVKSDKWNWFYINA |
| P07072.2 | I-TevII | 107 | >gi\|20141823\|sp\|P07072.2\|TEV2_BPT4 RecName: Full = Intron- |
| | | | associated endonuclease 2; AltName: Full = I-TevII |
| | | | MKWKLRKSLKIANSVAFTYMVRFPDKSFYIGFKKFKTIYGKDTNWKEYNSSSKLVKE |
| | | | KLKDYKAKWIILQVFDSYESALKHEEMLIRKYFNNEFILNKSIGGYKFNKYPDSEEH |
| | | | KQKLSNAHKGKILSLKHKDKIREKLIEHYKNNSRSEAHVKNNIGSRTAKKTVSIALK |
| | | | SGNKFRSFKSAAKFLKCSEEQVSNHPNVIDIKITIHPVPEYVKINDNIYKSFVDAAK |
| | | | DLKLHPSRIKDLCLDDNYPNYIVSYKRVEK |
| Q38419.1 | I-TevIII | 108 | >gi\|11387192\|sp\|Q38419.1\|TEV3_BPR03 RecName: Full = Intron- |
| | | | associated endonuclease 3; AltName: Full = I-TevIII |
| | | | MNYRKIWIDANGPIPKDSDGRTDEIHHKDGNRENNDLDNLMCLSIQEHYDIHLAQKD |
| | | | YQACHAIKLRMKYSPEEISELASKAAKSREIQIFNIPEVRAKNIASIKSKIENGTFH |
| | | | LLDGEIQRKSNLNRVALGIHNFQQAEHIAKVKERNIAAIKEGTHVFCGGKMQSETQS |
| | | | KRVNDGSHHFLSEDHKKRTSAKTLEMVKNGTHPAQKEITCDFCGHIGKGPGFYLKHN |
| | | | DRCKLNPNRIQLNCPYCDKKDLSPSTYKRWHGDNCKARFND |
| AAM00817.1 | I-TwoI | 109 | >gi\|19881200\|gb\|AAM00817.1\|AF485060_2 HNH endonuclease I- |
| | | | TwoI [Staphylococcus phage Twort] |
| | | | MEELWKEIPGFNSYMISNKGQVYSRKRNKILALRTDKNGYKRISIFNNEGKRILLGV |
| | | | HKLVLLGFKGINTEKPIPHHKNNIKDDNRLENLEWVTVSENTKHAYDIGALKSPRRV |
| | | | TCTLYYKGEPLSCYDSLFDLAKALKVSRSVIESPRNGLVLSTFEVKREPTIQGLPLN |
| | | | KEIFEHSLIKGLGNPPLKVYNEDETYYFLTLMDISKYFNESYSKVQRGYYKGKWKSY |
| | | | IIEHIDEYEYYKQTH |
| P11405.1 | R.MspI | 110 | >gi\|135239\|sp\|P11405.1\|T2M1_MORSP RecName: Full = Type-2 |
| | | | restriction enzyme MspI; Short = R.MspI; AltName: |
| | | | Full = Endonuclease MspI; AltName: Full = Type II restriction |
| | | | enzyme MspI |
| | | | MRTELLSKLYDDFGIDQLPHTQHGVTSDRLGKLYEKYILDIFKDIESLKKYNTNAFP |
| | | | QEKDISSKLLKALNLDLDNIIDVSSSDTDLGRTIAGGSPKTDATIRFTFHNQSSRLV |
| | | | PLNIKHSSKKKVSIAEYDVETICTGVGISDGELKELIRKHQNDQSAKLFTPVQKQRL |
| | | | TELLEPYRERFIRWCVTLRAEKSEGNILHPDLLIRFQVIDREYVDVTIKNIDDYVSD |
| | | | RIAEGSKARKPGFGTGLNWTYASGSKAKKMQFKG |
| R.MvaI | R.MvaI | 111 | >gi\|119392963\|gb\|AAM03024.2\|AF472612_1 R.MvaI [Kocuria |
| | | | varians] |
| | | | MSEYLNLLKEAIQNVVDGGWHETKRKGNTGIGKTFEDLLEKEEDNLDAPDFHDIEIK |
| | | | THETAAKSLLTLFTKSPTNPRGANTMLRNRYGKKDEYGNNILHQTVSGNRKTNSNSY |
| | | | NYDFKIDIDWESQVVRLEVFDKQDIMIDNSVYWSFDSLQNQLDKKLKYIAVISAESK |
| | | | IENEKKYYKYNSANLFTDLTVQSLCRGIENGDIKVDIRIGAYHSGKKKGKTHDHGTA |
| | | | FRINMEKLLEYGEVKVIV |
| CAA45962.1 | NucA | 112 | >gi\|39041\|emb\|CAA45962.1\|NucA [Nostoc sp. PCC 7120] |
| | | | MGICGKLGVAALVALIVGCSPVQSQVPPLTELSPSISVHLLLGNPSGATPTKLTPDN |
| | | | YLMVKNQYALSYNNSKGTANWVAWQLNSSWLGNAERQDNFRPDKTLPAGWVRVTPSM |
| | | | YSGSGYDRGHIAPSADRTKTTEDNAATFLMTNMMPQTPDNNRNTWGNLEDYCRELVS |
| | | | QGKELYIVAGPNGSLGKPLKGKVTVPKSTWKIVVVLDSPGSGLEGITANTRVIAVNI |
| | | | PNDPELNNDWRAYKVSVDELESLTGYDFLSNVSPNIQTSIESKVDN |
| P37994.2 | NucM | 113 | >gi\|313104150\|sp\|P37994.2\|NUCM_DICD3 RecName: |
| | | | Full = Nuclease nucM; Flags: Precursor |
| | | | MLRNLVIFAVLGAGLTTLAAAGQDINNFTQAKAAAAKIHQDAPGTFYCGCKINWQGK |
| | | | KGTPDLASCGYQVRKDANRASRIEWEHVVPAWQFGHQRQCWQDGGRKNCTKDDVYRQ |
| | | | IETDLHNLQPAIGEVNGDRGNFMYSQWNGGERQYGQCEMKIDFKSQLAEPPERARGA |
| | | | IARTYFYMRDRYNLNLSRQQTQLFDAWNKQYPATTWECTREKRIAAVQGNHNPYVQQ |
| | | | ACQP |
| AAF19759.1 | Vvn | 114 | >gi\|6635279\|gb\|AAF19759.1\|AF063303_1 nuclease precursor |
| | | | Vvn [Vibrio vulnificus] |
| | | | MKRLFIFIASFTAFAIQAAPPSSFSAAKQQAVKIYQDHPISFYCGCDIEWQGKKGIP |
| | | | NLETCGYQVRKQQTRASRIEWEHVVPAWQFGHHRQCWQKGGRKNCSKNDQQFRLMEA |
| | | | DLHNLTPAIGEVNGDRSNFNFSQWNGVDGVSYGRCEMQVNFKQRKVMPQTELRGSIA |
| | | | RTYLYMSQEYGFQLSKQQQQLMQAWNKSYPVDEWECTRDDRIAKIQGNHNPFVQQSC |
| | | | QTQ |
| AAF19759.1 | Vvn_CLS | 115 | >Vvn_CLS (variant of AAF19759.1) |
| (reference) | | | MASGAPPSSFSAAKQQAVKIYQDHPISFYCGCDIEWQGKKGIPNLETCGYQVRKQQT |
| | | | RASRIEWEHVVPAWQFGHHRQCWQKGGRKNCSKNDQQFRLMEADLHNLTPAIGEVNG |
| | | | DRSNFNFSQWNGVDGVSYGRCEMQVNFKQRKVMPPDRARGSIARTYLYMSQEYGFQL |
| | | | SKQQQQLMQAWNKSYPVDEWECTRDDRIAKIQGNHNPFVQQSCQTQGSSAD |
| P00644.1 | Staphylococcal | 116 | >gi\|128852\|sp\|P00644.1\|NUC_STAAU RecName: |
| | nuclease | | Full = Thermonuclease; Short = TNase; AltName: |
| | (NUC_STAAU) | | Full = Micrococcal nuclease; AltName: Full = Staphylococcal |
| | | | nuclease; Contains: RecName: Full = Nuclease B; Contains: |
| | | | RecName: Full = Nuclease A; Flags: Precursor |
| | | | MLVMTEYLLSAGICMAIVSILLIGMAISNVSKGQYAKRFFFFATSCLVLTLVVVSSL |
| | | | SSSANASQTDNGVNRSGSEDPTVYSATSTKKLHKEPATLIKAIDGDTVKLMYKGQPM |
| | | | TFRLLLVDTPETKHPKKGVEKYGPEASAFTKKMVENAKKIEVEFDKGQRTDKYGRGL |
| | | | AYIYADGKMVNEALVRQGLAKVAYVYKPNNTHEQHLRKSEAQAKKEKLNIWSEDNAD |
| | | | SGQ |
| P43270.1 | Staphylococcal | 117 | >gi\|1171859\|sp\|P43270.1\|NUC_STAHY RecName: |
| | nuclease | | Full = Thermonuclease; Short = TNase; AltName: |
| | (NUC_STAHY) | | Full = Micrococcal nuclease; AltName: Full = Staphylococcal |
| | | | nuclease; Flags: Precursor |
| | | | MKKITTGLIIVVAAIIVLSIQFMTESGPFKSAGLSNANEQTYKVIRVIDGDTIIVDK |
| | | | DGKQQNLRMIGVDTPETVKPNTPVQPYGKEASDFTKRHLTNQKVRLEYDKQEKDRYG |
| | | | RTLAYVWLGKEMFNEKLAKEGLARAKFYRPNYKYQERIEQAQKQAQKLKKNIWSN |
| P29769.1 | Micrococcal | 118 | >gi\|266681\|sp\|P29769.1\|NUC_SHIFL RecName: |
| | nuclease | | Full = Micrococcal nuclease; Flags: Precursor |
| | (NUC_SHIFL) | | MKSALAALRAVAAAVVLIVSVPAWADFRGEVVRILDGDTIDVLVNRQTIRVRLADID |
| | | | APESGQAFGSRARQRLADLTFRQEVQVTEKEVDRYGRTLGVVYAPLQYPGGQTQLTN |
| | | | INAIMVQEGMAWAYRYYGKPTDAQMYEYEKEARRQRLGLWSDPNAQEPWKWRRASKN |
| | | | ATN |
| P94492.1 | Endonuclease | 119 | >gi\|81345826\|sp\|\|YNCB_BACSU RecName: Full = Endonuclease |
| | yncB | | yncB; Flags: Precursor |
| | | | MKKILISMIAIVLSITLAACGSNHAAKNHSDSNGTEQVSQDTHSNEYNQTEQKAGTP |
| | | | HSKNQKKLVNVTLDRAIDGDTIKVIYNGKKDTVRYLLVDTPETKKPNSCVQPYGEDA |
| | | | SKRNKELVNSGKLQLEFDKGDRRDKYGRLLAYVYVDGKSVQETLLKEGLARVAYVYE |
| | | | PNTKYIDQFRLDEQEAKSDKLSIWSKSGYVTNRGFNGCVK |
| P00641.1 | Endodeoxyri | 120 | >gi\|119370\|sp\|P00641.1\|ENRN_BPT7 RecName: |
| | bonuclease I | | Full = Endodeoxyribonuclease I; AltName: |
| | (ENRN_BPT7) | | Full = Endodeoxyribonuclease I; Short = Endonuclease |
| | | | MAGYGAKGIRKVGAFRSGLEDKVSKQLESKGIKFEYEEWKVPYVIPASNHTYTPDFL |
| | | | LPNGIFVETKGLWESDDRKKHLLIREQHPELDIRIVFSSSRTKLYKGSPTSYGEFCE |
| | | | KHGIKFADKLIPAEWIKEPKKEVPFDRLKRKGGKK |
| Q53H47.1 | Metnase | 121 | >gi\|74740552\|sp\|Q53H47.1\|SETMR_HUMAN RecName: |
| | | | Full = Histone-lysine N-methyltransferase SETMAR; AltName: |
| | | | Full = SET domain and mariner transposase fusion gene- |
| | | | containing protein; Short = HsMar1; Short = Metnase; |
| | | | Includes: RecName: Full = Histone-lysine N- |
| | | | methyltransferase; Includes: RecName: Full = Mariner |
| | | | transposase Hsmar1 |
| | | | MAEFKEKPEAPTEQLDVACGQENLPVGAWPPGAAPAPFQYTPDHVVGPGADIDPTQI |
| | | | TFPGCICVKTPCLPGTCSCLRHGENYDDNSCLRDIGSGGKYAEPVFECNVLCRCSDH |
| | | | CRNRVVQKGLQFHFQVFKTHKKGWGLRTLEFIPKGRFVCEYAGEVLGFSEVQRRIHL |
| | | | QTKSDSNYIIAIREHVYNGQVMETFVDPTYIGNIGRFLNHSCEPNLLMIPVRIDSMV |
| | | | PKLALFAAKDIVPEEELSYDYSGRYLNLTVSEDKERLDHGKLRKPCYCGAKSCTAFL |
| | | | PFDSSLYCPVEKSNISCGNEKEPSMCGSAPSVFPSCKRLTLETMKMMLDKKQIRAIF |
| | | | LFEFKMGRKAAETTRNINNAFGPGTANERTVQWWFKKFCKGDESLEDEERSGRPSEV |
| | | | DNDQLRAIIEADPLTTTREVAEELNVNHSTVVRHLKQIGKVKKLDKWVPHELTENQK |
| | | | NRRFEVSSSLILRNHNEPFLDRIVTCDEKWILYDNRRRSAQWLDQEEAPKHFPKPIL |
| | | | HPKKVMVTIWWSAAGLIHYSFLNPGETITSEKYAQEIDEMNQKLQRLQLALVNRKGP |
| | | | ILLHDNARPHVAQPTLQKLNELGYEVLPHPPYSPDLLPTNYHVFKHLNNFLQGKRFH |
| | | | NQQDAENAFQEFVESQSTDFYATGINQLISRWQKCVDCNGSYFD |
| ABD15132.1 | Nb.BsrDI | 122 | >gi\|86757493\|gb\|ABD15132.1\| Nb.BsrDI [Geobacillus |
| | | | stearothermophilus] |
| | | | MTEYDLHLYADSFHEGHWCCENLAKIAQSDGGKHQIDYLQGFIPRHSLIFSDLIINI |
| | | | TVFGSYKSWKHLP |
| | | | KQIKDLLFWGKPDFIAYDPKNDKILFAVEETGAVPTGNQALQRCERIYGSARKQIPF |
| | | | WYLLSEFGQHKDGGTRRDSIWPTIMGLKLTQLVKTPSIILHYSDINNPEDYNSGNGL |
| | | | KFLFKSLLQIIINYCTLKNPLKGMLELLSIQYENMLEFIKSQWKEQIDFLPGEEILN |
| | | | TKTKELARMYASLAIGQTVKIPEELFNWPRTDKVNFKSPQGLIKYDELCYQLEKAVG |
| | | | SKKAYCLSNNAGAKPQKLESLKEWINSQKKLFDKAPKLTPPAEFNMKLDAFPVTSNN |
| | | | NYYVTTSKNILYLFDYWKDLRIAIETAFPRLKGKLPTDIDEKPALIYICNSVKPGRL |
| | | | FGDPFTGQLSAFSTIFGKKNIDMPRIVVAYYPHQIYSQALPKNNKSNKGITLKKELT |
| | | | DFLIFHGGVVVKLNEGKAY |
| ABD15133.1 | BsrDI A | 123 | >gi\|86757494\|gb\|ABD15133.1\| BsrDI A [Geobacillus |
| | | | stearothermophilus] |
| | | | MTDYRYSFELSEEIARWAFEIKTKNTDWFVAFSNPTAGPWKRVMAIDKASNREGEVH |
| | | | RFGREDERPDIILVNDNISLILILEAKEKLNQLISKSQVDKSVDVFLTLSSILKEKS |
| | | | DNNYWGDRTKYINVLGILWGSEQETSQKDIDNAFRVYRDSLVKNLKEINPTPTNICT |
| | | | DILVGVESIKNKKEEISIKIHVSNIYAEIYPKFTGKHLLEKLAVLN |
| ABN42182.1 | Nt.BspD6I | 124 | >gi\|125396996\|gb\|ABN42182.1\| heterodimeric restriction |
| | (R.BspD6I | | endonuclease R.BspD6I large subunit [Bacillus sp. D6] |
| | large subunit) | | MAKKVNWYVSCSPRSPEKIQPELKVLANFEGSYWKGVKGYKAQEAFAKELAALPQFL |
| | | | GTTYKKEAAFSTRDRVAPMKTYGFVFVDEEGYLRITEAGKMLANNRRPKDVFLKQLV |
| | | | KWQYPSFQHKGKEYPEEEWSINPLVFVLSLLKKVGGLSKLDIAMFCLTATNNNQVDE |
| | | | IAEEIMQFRNEREKIKGQNKKLEFTENYFFKRFEKIYGNVGKIREGKSDSSHKSKIE |
| | | | TKMRNARDVADATTRYFRYTGLFVARGNQLVLNPEKSDLIDEIISSSKVVKNYTRVE |
| | | | EFHEYYGNPSLPQFSFETKEQLLDLAHRIRDENTRLAEQLVEHFPNVKVEIQVLEDI |
| | | | YNSLNKKVDVETLKDVIYHAKELQLELKKKKLQADFNDPRQLEEVIDLLEVYHEKKN |
| | | | VIEEKIKARFIANKNTVFEWLTWNGFIILGNALEYKNNFVIDEELQPVTHAAGNQPD |
| | | | MEIIYEDFIVLGEVTTSKGATQFKMESEPVTRHYLNKKKELEKQGVEKELYCLFIAP |
| | | | EINKNTFEEFMKYNIVQNTRIIPLSLKQFNMLLMVQKKLIEKGRRLSSYDIKNLMVS |
| | | | LYRTTIECERKYTQIKAGLEETLNNWVVDKEVRF |
| ABN42183.1 | ss.BspD6I | 125 | >gi\|125396997\|gb\|ABN42183.1\| heterodimeric restriction |
| | (R.BspD6I | | endonuclease R.BspD6I small subunit |
| | small | | [Bacillus sp. D6] |
| | subunit) | | MQDILDFYEEVEKTINPPNYFEWNTYRVFKKLGSYKNLVPNFKLDDSGHPIGNAIPG |
| | | | VEDILVEYEHFSILIECSLTIGEKQLDYEGDSVVRHLQEYKKKGIEAYTLFLGKSID |
| | | | LSFARHIGFNKESEPVIPLTVDQFKKLVTQLKGDGEHFNPNKLKEILIKLLRSDLGY |
| | | | DQAEEWLTFIEYNLK |
| AAK27215.1 | R.PleI | 126 | >gi\|13448813\|gb\|AAK27215.1\|AF355461_2 restriction |
| | | | endonuclease R.PleI [Paucimonas lemoignei] |
| | | | MAKPIDSKVLFITTSPRTPEKMVPEIELLDKNFNGDVWNKDTQTAFMKILKEESFFD |
| | | | GEGKNDPAFSARDRINRAPKSLGFVILTPKLSLTDAGVELIKAKRKDDIFLRQMLKF |
| | | | QLPSPYHKLSDKAALFYVKPYLEIFRLVRHFGSLTFDELMIFGLQIIDFRIFNQIVD |
| | | | KIEDFRVGKIENKGRYKTYKKERFEEELGKIYKDELFGLTEASAKTLITKKGNNMRD |
| | | | YADACVRYLRATGMVNVSYQGKSLSIVQEKKEEVDFFLKNTEREPCFINDEASYVSY |
| | | | LGNPNYPKLFVDDVDRIKKKLRFDFKKTNKVNALTLPELKEELENEILSRKENILKS |
| | | | QISDIKNFKLYEDIQEVFEKIENDRTLSDAPLMLEWNTWRAMTMLDGGEIKANLKFD |
| | | | DFGSPMSTAIGNMPDIVCEYDDFQLSVEVTMASGQKQYEMEGEPVSRHLGKLKKSSE |
| | | | KPVYCLFIAPKINPSSVAHFFMSHKVDIEYYGGKSLIIPLELSVFRKMIEDTFKASY |
| | | | IPKSDNVHKLFKNFASIADEAGNEKVWYEGVKRTAMNWLSLS |
| AAK39546.1 | MlyI | 127 | >gi\|13786046\|gb\|AAK39546.1\|AF355462_2 MlyIR [Micrococcus |
| | | | lylae] |
| | | | MASLSKTKHLFGFTSPRTIEKIIPELDILSQQFSGKVWGENQINFFDAIFNSDFYEG |
| | | | TTYPQDPALAARDRITRAPKALGFIQLKPVIQLTKAGNQLVNQKRLPELFTKQLLKF |
| | | | QLPSPYHTQSPTVNFNVRPYLELLRLINELGSISKTEIALFFLQLVNYNKFDEIKNK |
| | | | ILKFRETRKNNRSVSWKTYVSQEFEKQISIIFADEVTAKNFRTRESSDESFKKFVKT |
| | | | KEGNMKDYADAFFRYIRGTQLVTIDKNLHLKISSLKQDSVDFLLKNTDRNALNLSLM |
| | | | EYENYLFDPDQLIVLEDNSGLINSKIKQLDDSINVESLKIDDAKDLLNDLEIQRKAK |
| | | | TIEDTVNHLKLRSDIEDILDVFAKIKKRDVPDVPLFLEWNIWRAFAALNHTQAIEGN |
| | | | FIVDLDGMPLNTAPGKKPDIEINYGSFSCIVEVTMSSGETQFNMEGSSVPRHYGDLV |
| | | | RKVDHDAYCIFIAPKVAPGTKAHFFNLNRLSTKHYGGKTKIIPMSLDDFICFLQVGI |
| | | | THNFQDINKLKNWLDNLINFNLESEDEEIWFEEIISKISTWAI |
| YP_004134094.1 | AlwI | 128 | >gi\|319768594\|ref\|YP_004134094.1\| restriction |
| | | | endonuclease, type II, AlwI [Geobacillus sp.Y412MC52] |
| | | | MNKKNTRKVWFITRPERDPRFHQEALLALQKATDDFRLKWAGNREVHKRYEEELANM |
| | | | GIKRNNVSHDGSGGRTWMAMLKTFSYCYVDDDGYIRLTKVGEKLIQGEKVYENTRKQ |
| | | | VLTLQYPNAYFLEPGFRPKFDEGFRIRPVLFLIKLANDERLDFYVTKEEITYFAMTA |
| | | | QKDSQLDEIVHKILAFRKAGPREREEMKQDIAAKFDHRERSDKGARDFYEAHSDVAH |
| | | | TFMLISDYTGLVEYIRGKALKGDSSKINEIKQEIAEIEKRYPFNTRYMISLERMAEN |
| | | | SGLDVDSYKASRYGNIKPAANSSKLRAKAERILAQFPSIESMSKEEIAGALQKYLSP |
| | | | RDIEKVIHEIVENKDDFEGINSDFVETYLNEKDNLAFEDKTGQIFSALGFDVAMRPK |
| | | | AKNGERTEIEIIARYGGSKFGIIDAKNYAGKFPLSSSLVSHMASEYIPNYTGYEGKE |
| | | | LTFFGYVTANDFSGERNLEKISDKAKRITGNPISGFLVTARTLLGFLDYCIENDVPL |
| | | | EDRAELFVKAVKNKGYKSLEALLRELKETI |
| AAY97906.1 | Mva12691 | 129 | >gi\|68480350\|gb\|AAY97906.1\|Mva1269I restriction |
| | | | endonuclease [Kocuria varians] |
| | | | MYLNTAVFNIYGDNIVECSRAFHYILEGFKLANISITQEYDLQNITTPKFCIYTDKF |
| | | | RYIFIFIPGTSASRWNKDIYKELVLNNGGPLKEGADAIITRIFSEDSELVLASMEFS |
| | | | AALPAGNNTWQRSGRAYSLTAANIPYFYIVQLGGKEIKKGKDGKSDKFATRLPNPAL |
| | | | SLSFTLNTIKKPAPSLIVYDQAPEADSAISDLYSNCYGIDDFSLYLFKLITEENNLH |
| | | | ELKNIYNKNVEFLQLRSVDEKGKNFSGKDYKYIFEHKDPYKGLTEVVKERKIPWKKK |
| | | | TATKTFENFPLRNQAPIFRLIDFLSTKSYGIVSKDSLPLTFIPSEHRVEVANYICNQ |
| | | | LYIDKVSDEFVKWIYKKEDLAICIINGFKPGGDDSRPDRGLPPFTKMLTNLDILTLM |
| | | | FGPAPPTQWDYLDSDPEKLNKTNGLWQSIFAFSDAILVDSSTRDNNKFVYNAYLKEH |
| | | | WVVQREKKESNTPISYFPKSVGEHDVDTSLHILFTYIGKHFESACNPPGGDWSGVSL |
| | | | LKNNIEYRWTSMYRVSQDGTKRPDHIYQLVYNSTDTLLLIESKGIKNDLLKSKEANV |
| | | | GIGMINYLKNLMARDYTAVKKDGEWKNIHGQMTLDKFLTFSAVAYLFTTDFDNEYTS |
| | | | AAELLVHSNTQLAFALEIKEKNSVMHIFTANTVAYNFAEYLLETMRNSHLPLKIYKP |
| | | | I |
| ADR72996.1 | BsrI | 130 | >gi\|313667100\|gb\|ADR72996.1\| BsrI [Geobacillus |
| | | | stearothermophilus] |
| | | | MRNIRIYSEVKEQGIFFKEVIQSVLEKANVEVVLVNSAMLDYSDVSVISLIRNQKKF |
| | | | DLLVSEVRDKREIPIVMVEFSTAVTTDDHELQRADAMFWAYKYKIPYLKISPMEKKS |
| | | | QTADDKFGGGRLLSVNDQIIHMYRTDGVMYHIEWESMDNSAYVKNAELYPSCPDCAP |
| | | | ELASLFRCLLETIEKCENIEDYYRILLDKLGKQKVAVKWGNFREEKTLEQWKHEKFD |
| | | | LLERFSKSSSRMEYDKDKKELKIKVNRYGHAMDPERGILAFWKLVLGDEWKIVAEFQ |
| | | | LQRKTLKGRQSYQSLFDEVSQEEKLMNIASEIIKNGNVISPDKAIEIHKLATSSTMI |
| | | | STIDLGTPERKYITDDSLKGYLQHGLITNIYKNLLYYVDEIRFTDLQRKTIASLTWN |
| | | | KEIVNDYYKSLMDQLLDKNLRVLPLTSIKNISEDLITWSSKEILINLGYKILAASYP |
| | | | EAQGDRCILVGPTGKKTERKFIDLIAISPKSKGVILLECKDKLSKSKDDCEKMNDLL |
| | | | NHNYDKVTKLINVLNINNYNYNNIIYTGVAGLIGRKNVDNLPVDFVIKFKYDAKNLK |
| | | | LNWEINSDILGKHSGSFSMEDVAVVRKRS |
| AAL86024.1 | BsmI | 131 | >gi\|19347662\|gb\|AAL86024.1\| BsmI [Geobacillus |
| | | | stearothermophilus] |
| | | | MNVFRIHGDNIIECERVIDLILSKINPQKVKRGFISLSCPFIEIIFKEGHDYFHWRF |
| | | | DMFPGFNKNTNDRWNSNILDLLSQKGSFLYETPDVIITSLNNGKEEILMAIEFCSAL |
| | | | QAGNQAWQRSGRAYSVGRTGYPYIYIVDFVKYELNNSDRSRKNLRFPNPAIPYSYIS |
| | | | HSKNTGNFIVQAYFRGEEYQPKYDKKLKFFDETIFAEDDIADYIIAKLQHRDTSNIE |
| | | | QLLINKNLKMVEFLSKNTKNDNNFTYSEWESIYNGTYRITNLPSLGRFKFRKKIAEK |
| | | | SLSGKVKEFNNIVQRYSVGLASSDLPFGVIRKESRNDFINDVCKLYNINDMKIIKEL |
| | | | KEDADLIVCMLKGFKPRGDDNRPDRGALPLVAMLAGENAQIFTFIYGPLIKGAINLI |
| | | | DQDINKLAKRNGLWKSFVSLSDFIVLDCPIIGESYNEFRLIINKNNKESILRKTSKQ |
| | | | QNILVDPTPNHYQENDVDTVIYSIFKYIVPNCFSGMCNPPGGDWSGLSIIRNGHEFR |
| | | | WLSLPRVSENGKRPDHVIQILDLFEKPLLLSIESKEKPNDLEPKIGVQLIKYIEYLF |
| | | | DFTPSVQRKIAGGNWEFGNKSLVPNDFILLSAGAFIDYDNLTENDYEKIFEVTGCDL |
| | | | LIAIKNQNNPQKWVIKFKPKNTIAEKLVNYIKLNFKSNIFDTGFFHIEG |
| AD124225.1 | Nb.BtsCI | 132 | >gi\|297185870\|gb\|ADI24225.1\| BtsCI bottom-strand nicking |
| | | | enzyme variant [synthetic construct] |
| | | | MKRILYLLTEERPKINIIHQIINLEYKATLHFGAKIVPVMNEENKFTFIYHVKGIEV |
| | | | EGFDAVLIKIVSGHSSFVDYLVFDSNDLKPEKNTITLFDLDQYELDLSYYFGKGWIV |
| | | | RIPSPSDLPKYVVEETKTDDHESRNTNAYQRSSKFVFCELYYGKEVKKYMLYDISDG |
| | | | RTLSGTDTHNFGMRMLVTNNVNLVGVPNMYLPFTDIKEFINEKNRIADNGPSHNVPI |
| | | | RLKLDKEKNVIYISAKLDKGNGKNKNKISNDPNIGAVAIISATLRNLNWKGDIEIIN |
| | | | HNLLPSSISSRSNGNKLLYIMKKLGVRFNNINVNWNNIKNNINYFFYNITSEKIVSI |
| | | | YYHLYVEDKLSNARVIFDNHAGCGKSYFRTLNNKIIPVGKEIPLPALVIFDSDQNIV |
| | | | KVIAAAKAENVYNGVEQLSTFDKFIESYINKYYPGAAVECSVITWGKSSNPYVSFYL |
| | | | DKDGSAVFL |
| AD124224.1 | Nt.BtsCI | 133 | >gi\|297185868\|gb\|ADI24224.1\| BtsCI top-strand nicking |
| | | | enzyme variant [synthetic construct] |
| | | | MKRILYLLTEERPKINIIHQIINLEYKATLHFGAKIVPVMNEENKFTFIYHVKGIEV |
| | | | EGFDAVLIKIVSGHSSFVDYLVFDSNDLKPEKNTITLFDLDQYELDLSYYFGKGWIV |
| | | | RIPSPSDLPKYVVFETKTDDHESRNTNAYQRSSKFVFCELYYGKEVKKYMLYDISDG |
| | | | RTLSGTDTHNFGMRMLVTNNVNLVGVPNMYLPFTDIKEFINEKNRIADNGPSHNVPI |
| | | | RLKLDKEKNVIYISAKLDKGNGKNKNKISNDPNIGAVAIISATLRNLNWKGDIEIIN |
| | | | HNLLPSSISSRSNGNKLLYIMKKLGVRFNNINVNWNNIKNNINYFFYNITSEKIVSI |
| | | | YYHLYVEDKLSNARVIFDNHAGCGKSYFRTLNNKIIPVGKEIPLPDLVIFDSDQNIV |
| | | | KVIEAEKAENVYNGVEQLSTFDKFIESYINKYYPGAAVECSVITWGKSSNPYVSFYL |
| | | | DKDGSAVFL |
| | | | >gi\|85720924\|gb\|ABC75874.1\| R1.BtsI [Geobacillus |
| | | | thermoglucosidasius] |
| | | | MKITEGIVHVAMRHFLKSNGWKLIAGQYPGGSDDELTALNIVDPVVARDNSPDPRRH |
| | | | SLGKIVPDLIAYKNDDLLVIEAKPKYSQDDRDKLLYLLSERKHDFYAALEKFATERN |
| | | | HPELLPVSKLNIIPGLAFSASENKFKKDPGFVYIRVSGIFEAFMEGYDWG |
| ABC75874.1 | R1.BtsI | 134 | >gi\|85720924\|gb\|ABC75874.1\| R1.BtsI [Geobacillus |
| | | | thermoglucosidasius] |
| | | | MKITEGIVHVAMRHFLKSNGWKLIAGQYPGGSDDELTALNIVDPVVARDNSPDPRRH |
| | | | SLGKIVPDLIAYKNDDLLVIEAKPKYSQDDRDKLLYLLSERKHDFYAALEKFATERN |
| | | | HPELLPVSKLNIIPGLAFSASENKFKKDPGFVYIRVSGIFEAFMEGYDWG |
| ABC75876.1 | R2.BtsI | 135 | >gi\|85720926\|gb\|ABC75876.1\| R2.BtsI [Geobacillus |
| | | | thermoglucosidasius] |
| | | | MQIEQLMKSLTIYFDDIQEGLWFKNLHPLLESASLEAITGSLKRNPNLADVLKYDRP |
| | | | DIILTLNQTPILVIERTIEVPSGHNVGQRYGRLAAASEAGVPLVYFGPYAARKHGGA |
| | | | TEGPRYMNLRLFYALDVMQKVNGSAITTINWPVDQNFEILQDPSKDKRMKEYLEMFF |
| | | | DNLLKYGIAGINLAIRNSSFQAEQLAEREKFVETMITNPEQYDVPPDSVQILNAERF |
| | | | FNELGISENKRIICDEVVLYQVGMTYVRSDPYTGMALLYKYLYILGSERNRCLILKF |
| | | | PNITTDMWKKVAFGSRERKDVRIYRSVSDGILFADGYLSKEEL |
| AAX14652.1 | BbvCI subunit | 136 | >gi\|60202520\|gb\|AAX14652.1\| BbvCI endonuclease subunit 1 |
| | | | [Brevibacillus brevis] |
| | | | MINEDFFIYEQLSHKKNLEQKGKNAFDEETEELVRQAKSGYHAFIEGINYDEVTKLD |
| | | | LNSSVAALEDYISIAKEIEKKHKMFNWRSDYAGSIIPEFLYRIVHVATVKAGLKPIF |
| | | | STRNTIIEISGAAHREGLQIRRKNEDFALGFHEVDVKIASESHRVISLAVACEVKTN |
| | | | IDKNKLNGLDFSAERMKRTYPGSAYFLITETLDFSPDENHSSGLIDEIYVLRKQVRT |
| | | | KNRVQKAPLCPSVFAELLEDILEISYRASNVKGHVYDRLEGGKLIRV |
| AAX14653.1 | BbvCI | 137 | >gi\|60202521\|gb\|AAX14653.1\| BbvCI endonuclease subunit 2 |
| | subunit2 | | [Brevibacillus brevis] |
| | | | MFNQFNPLVYTHGGKLERKSKKDKTASKVFEEFGVMEAYNCWKEASLCIQQRDKDSV |
| | | | LKLVAALNTYKDAVEPIFDSRLNSAQEVLQPSILEEFFEYLFSRIDSIVGVNIPIRH |
| | | | PAKGYLSLSFNPHNIETLIQSPEYTVRAKDHDFIIGGSAKLTIQGHGGEGETTNIVV |
| | | | PAVAIECKRYLERNMLDECAGTAERLKRATPYCLYFVVAEYLKLDDGAPELTEIDEI |
| | | | YILRHQRNSERNKPGFKPNPIDGELIWDLYQEVMNHLGKIWWDPNSALQRGKVFNRP |
| CAA74998.1 | Bpu10I alpha | 138 | >gi\|2894388\|emb\|CAA74998.1\| Bpu10I restriction |
| | subunit | | endonuclease alpha subunit [Bacillus pumilus] |
| | | | MGVEQEWIKNITDMYQSPELIPSHASNLLHQLKREKRNEKLKKALEIITPNYISYIS |
| | | | ILLNNHNMTRKEIVILVDALNEYMNTLRHPSVKSVFSHQADFYSSVLPEFFNLLFRN |
| | | | LIKGLNEKIKVNSQKDIIIDCIFDPYNEGRVVFKKKRVDVAIILKNKFVFNNVEISD |
| | | | FAIPLVAIEIKTNLDKNMLSGIEQSVDSLKETFPLCLYYCITELADFAIEKQNYAST |
| | | | HIDEVFILRKQKRGPVRRGTPLEVVHADLILEVVEQVGEHLSKFKDPIKTLKARMTE |
| | | | GYLIKGKGK |
| CAA74999.1 | Bpu10I beta | 139 | >gi\|2894389\|emb\|CAA74999.1\| Bpu10I restriction |
| | subunit | | endonuclease beta subunit [Bacillus pumilus] |
| | | | MTQIDLSNTKHGSILFEKQKNVKEKYLQQAYKHYLYFRRSIDGLEITNDEAIFKLTQ |
| | | | AANNYRDNVLYLFESRPNSGQEAFRYTILEEFFYHLFKDLVKKKFNQEPSSIVMGKA |
| | | | NSYVSLSFSPESFLGLYENPIPYIHTKDQDFVLGCAVDLKISPKNELNKENETEIVV |
| | | | PVIAIECKTYIERNMLDSCAATASRLKAAMPYCLYIVASEYMKMDQAYPELTDIDEV |
| | | | FILCKASVGERTALKKKGLPPHKLDENLMVELFHMVERHLNRVWWSPNEALSRGRVI |
| | | | GRP |
| ABM69266.1 | BmrI | 140 | >gi\|123187377\|gb\|ABM69266.1\| BmrI [Bacillus megaterium] |
| | | | MNYFSLHPNVYATGRPKGLINMLESVWISNQKPGDGTMYLISGFANYNGGIRFYETF |
| | | | TEHINHGGKVIAILGGSTSQRLSSKQVVAELVSRGVDVYIINRKRLLHAKLYGSSSN |
| | | | SGESLVVSSGNFTGPGMSQNVEASLLLDNNTTSSMGFSWNGMVNSMLDQKWQIHNLS |
| | | | NSNPTSPSWNLLYDERTTNLTLDDTQKVTLILTLGHADTARIQAAPKSKAGEGSQYF |
| | | | WLSKDSYDFFPPLTIRNKRGTKATYSCLINMNYLDIKYIDSECRVTFEAENNFDFRL |
| | | | GTGKLRYTNVAASDDIAAITRVGDSDYELRIIKKGSSNYDALDSAAVNFIGNRGKRY |
| | | | GYIPNDEFGRIIGAKF |
| CAC12783.1 | BfiI | 141 | >gi\|10798463\|emb\|CAC12783.1\| restriction endonuclease |
| | | | BfiI [Bacillus firmus] |
| | | | MNFFSLHPNVYATGRPKGLIGMLENVWVSNHTPGEGTLYLISGFSNYNGGVRFYETF |
| | | | TEHINQGGRVIAILGGSTSQRLSSRQVVEELLNRGVEVHIINRKRILHAKLYGTSNN |
| | | | LGESLVVSSGNFTGPGMSQNIEASLLLDNNTTQSMGFSWNDMISEMLNQNWHIHNMT |
| | | | NATDASPGWNLLYDERTTNLTLDETERVTLIVTLGHADTARIQAAPGTTAGQGTQYF |
| | | | WLSKDSYDFFPPLTIRNRRGTKATYSSLINMNYIDINYTDTQCRVTFEAENNFDFRL |
| | | | GTGKLRYTGVAKSNDIAAITRVGDSDYELRIIKQGTPEHSQLDPYAVSFIGNRGKRF |
| | | | GYISNEEFGRIIGVTF |
| Q9UQ84.2 | hExoI | 142 | >gi\|85700954\|sp\|Q9UQ84.2\|EXO1_HUMAN RecName: |
| | (EXO1_HUMAN) | | Full = Exonuclease 1; Short = hExo1; AltName: |
| | | | Full = Exonuclease I; Short = hExoI |
| | | | MGIQGLLQFIKEASEPIHVRKYKGQVVAVDTYCWLHKGAIACAEKLAKGEPTDRYVG |
| | | | FCMKFVNMLLSHGIKPILVFDGCTLPSKKEVERSRRERRQANLLKGKQLLREGKVSE |
| | | | ARECFTRSINITHAMAHKVIKAARSQGVDCLVAPYEADAQLAYLNKAGIVQAIITED |
| | | | SDLLAFGCKKVILKMDQFGNGLEIDQARLGMCRQLGDVFTEEKFRYMCILSGCDYLS |
| | | | SLRGIGLAKACKVLRLANNPDIVKVIKKIGHYLKMNITVPEDYINGFIRANNTFLYQ |
| | | | LVFDPIKRKLIPLNAYEDDVDPETLSYAGQYVDDSIALQIALGNKDINTFEQIDDYN |
| | | | PDTAMPAHSRSHSWDDKTCQKSANVSSIWHRNYSPRPESGTVSDAPQLKENPSTVGV |
| | | | ERVISTKGLNLPRKSSIVKRPRSAELSEDDLLSQYSLSFTKKTKKNSSEGNKSLSFS |
| | | | EVFVPDLVNGPTNKKSVSTPPRTRNKFATFLQRKNEESGAVVVPGTRSRFFCSSDST |
| | | | DCVSNKVSIQPLDETAVTDKENNLHESEYGDQEGKRLVDTDVARNSSDDIPNNHIPG |
| | | | DHIPDKATVFTDEESYSFESSKFTRTISPPTLGTLRSCFSWSGGLGDFSRTPSPSPS |
| | | | TALQQFRRKSDSPTSLPENNMSDVSQLKSEESSDDESHPLREEACSSQSQESGEFSL |
| | | | QSSNASKLSQCSSKDSDSEESDCNIKLLDSQSDQTSKLRLSHFSKKDTPLRNKVPGL |
| | | | YKSSSADSLSTTKIKPLGPARASGLSKKPASIQKRKHHNAENKPGLQIKLNELWKNF |
| | | | GFKKDSEKLPPCKKPLSPVRDNIQLTPEAEEDIFNKPECGRVQRAIFQ |
| P39875.2 | Yeast ExoI | 143 | >gi\|1706421\|sp\|P39875.2\|EXO1_YEAST RecName: |
| | (EXO1_YEAST) | | Full = Exodeoxyribonuclease 1; AltName: |
| | | | Full = Exodeoxyribonuclease I; Short = EXO I; |
| | | | Short = Exonuclease I; AltName: Full = Protein DHS1 |
| | | | MGIQGLLPQLKPIQNPVSLRRYEGEVLAIDGYAWLHRAACSCAYELAMGKPTDKYLQ |
| | | | FFIKRFSLLKTFKVEPYLVFDGDAIPVKKSTESKRRDKRKENKAIAERLWACGEKKN |
| | | | AMDYFQKCVDITPEMAKCIICYCKLNGIRYIVAPFEADSQMVYLEQKNIVQGIISED |
| | | | SDLLVFGCRRLITKLNDYGECLEICRDNFIKLPKKFPLGSLTNEEIITMVCLSGCDY |
| | | | TNGIPKVGLITAMKLVRRFNTIERIILSIQREGKLMIPDTYINEYEAAVLAFQFQRV |
| | | | FCPIRKKIVSLNEIPLYLKDTESKRKRLYACIGFVIHRETQKKQIVHFDDDIDHHLH |
| | | | LKIAQGDLNPYDFHQPLANREHKLQLASKSNIEFGKTNTTNSEAKVKPIESFFQKMT |
| | | | KLDHNPKVANNIHSLRQAEDKLTMAIKRRKLSNANVVQETLKDTRSKFFNKPSMTVV |
| | | | ENFKEKGDSIQDFKEDTNSQSLEEPVSESQLSTQIPSSFITTNLEDDDNLSEEVSEV |
| | | | VSDIEEDRKNSEGKTIGNEIYNTDDDGDGDTSEDYSETAESRVPTSSTTSFPGSSQR |
| | | | SISGCTKVLQKFRYSSSFSGVNANRQPLFPRHVNQKSRGMVYVNQNRDDDCDDNDGK |
| | | | NQITQRPSLRKSLIGARSQRIVIDMKSVDERKSFNSSPILHEESKKRDIETTKSSQA |
| | | | RPAVRSISLLSQFVYKGK |
| BAJ43803.1 | E. coli ExoI | 144 | >gi\|315136644\|dbj\|BAJ43803.1\| exonuclease I [Escherichia |
| | | | coli DH1] |
| | | | MMNDGKQQSTFLFHDYETFGTHPALDRPAQFAAIRTDSEFNVIGEPEVFYCKPADDY |
| | | | LPQPGAVLITGITPQEARAKGENEAAFAARIHSLFTVPKTCILGYNNVRFDDEVTRN |
| | | | IFYRNFYDPYAWSWQHDNSRWDLLDVMRACYALRPEGINWPENDDGLPSFRLEHLTK |
| | | | ANGIEHSNAHDAMADVYATIAMAKLVKTRQPRLFDYLFTHRNKHKLMALIDVPQMKP |
| | | | LVHVSGMFGAWRGNTSWVAPLAWHPENRNAVIMVDLAGDISPLLELDSDTLRERLYT |
| | | | AKTDLGDNAAVPVKLVHINKCPVLAQANTLRPEDADRLGINRQHCLDNLKILRENPQ |
| | | | VREKVVAIFAEAEPFTPSDNVDAQLYNGFFSDADRAAMKIVLETEPRNLPALDITFV |
| | | | DKRIEKLLFNYRARNFPGTLDYAEQQRWLEHRRQVFTPEFLQGYADELQMLVQQYAD |
| | | | DKEKVALLKALWQYAEEIV |
| Q9BQ50.1 | Human TREX2 | 145 | >gi\|47606206\|sp\|Q9BQ50.1\|TREX2_HUMAN RecName: Full = Three |
| | | | prime repair exonuclease 2; AltName:Full = 3′-5′ |
| | | | exonuclease TREX2 |
| | | | MGRAGSPLPRSSWPRMDDCGSRSRCSPTLCSSLRTCYPRGNITMSEAPRAETFVFLD |
| | | | LEATGLPSVEPEIAELSLFAVHRSSLENPEHDESGALVLPRVLDKLTLCMCPERPFT |
| | | | AKASEITGLSSEGLARCRKAGFDGAVVRTLQAFLSRQAGPICLVAHNGFDYDFPLLC |
| | | | AELRRLGARLPRDTVCLDTLPALRGLDRAHSHGTRARGRQGYSLGSLFHRYFRAEPS |
| | | | AAHSAEGDVHTLLLIFLHRAAELLAWADEQARGWAHIEPMYLPPDDPSLEA |
| Q91XB0.2 | Mouse TREX1 | 146 | >gi\|47606196\|sp\|Q91XB0.2\|TREX1 MOUSE RecName: Full = Three |
| | | | prime repair exonuclease 1; AltName: Full = 3′-5′ |
| | | | exonuclease TREX1 |
| | | | MGSQTLPHGHMQTLIFLDLEATGLPSSRPEVTELCLLAVHRRALENTSISQGHPPPV |
| | | | PRPPRVVDKLSLCIAPGKACSPGASEITGLSKAELEVQGRQRFDDNLAILLRAFLQR |
| | | | QPQPCCLVAHNGDRYDFPLLQTELARLSTPSPLDGTFCVDSIAALKALEQASSPSGN |
| | | | GSRKSYSLGSIYTRLYWQAPTDSHTAEGDVLTLLSICQWKPQALLQWVDEHARPFST |
| | | | VKPMYGTPATTGTTNLRPHAATATTPLATANGSPSNGRSRRPKSPPPEKVPEAPSQE |
| | | | GLLAPLSLLTLLTLAIATLYGLFLASPGQ |
| Q9NSU2.1 | Human TREX1 | 147 | >gi\|47606216\|sp\|Q9NSU2.1\|TREX1_HUMAN RecName: Full = Three |
| | | | prime repair exonuclease 1; AltName: Full = 3′-5′ |
| | | | exonuclease TREX1; AltName: Full = DNase III |
| | | | MGPGARRQGRIVQGRPEMCFCPPPTPLPPLRILTLGTHTPTPCSSPGSAAGTYPTMG |
| | | | SQALPPGPMQTLIFFDMEATGLPFSQPKVTELCLLAVHRCALESPPTSQGPPPTVPP |
| | | | PPRVVDKLSLCVAPGKACSPAASEITGLSTAVLAAHGRQCFDDNLANLLLAFLRRQP |
| | | | QPWCLVAHNGDRYDFPLLQAELAMLGLTSALDGAFCVDSITALKALERASSPSEHGP |
| | | | RKSYSLGSIYTRLYGQSPPDSHTAEGDVLALLSICQWRPQALLRWVDAHARPFGTIR |
| | | | PMYGVTASARTKPRPSAVTTTAHLATTRNTSPSLGESRGTKDLPPVKDPGALSREGL |
| | | | LAPLGLLAILTLAVATLYGLSLATPGE |
| Q9BG99.1 | Bovine TREX1 | 148 | >gi\|47606205\|sp\|Q9BG99.1\|TREX1_BOVIN RecName: Full = Three |
| | | | prime repair exonuclease 1; AltName: Full = 3′-5′ |
| | | | exonuclease TREX1 |
| | | | MGSRALPPGPVQTLIFLDLEATGLPFSQPKITELCLLAVHRYALEGLSAPQGPSPTA |
| | | | PVPPRVLDKLSLCVAPGKVCSPAASEITGLSTAVLAAHGRRAFDADLVNLIRTFLQR |
| | | | QPQPWCLVAHNGDRYDFPLLRAELALLGLASALDDAFCVDSIAALKALEPTGSSSEH |
| | | | GPRKSYSLGSVYTRLYGQAPPDSHTAEGDVLALLSVCQWRPRALLRWVDAHAKPFST |
| | | | VKPMYVITTSTGTNPRPSAVTATVPLARASDTGPNLRGDRSPKPAPSPKMCPGAPPG |
| | | | EGLLAPLGLLAFLTLAVAMLYGLSLAMPGQ |
| AAH91242.1 | Rat TREX1 | 149 | >gi\|60688197\|gb\|AAH91242.1\| Trex1 protein [Rattus |
| | | | norvegicus] |
| | | | MGSQALPHGHMQTLIFLDLEATGLPYSQPKITELCLLAVHRHALENSSMSEGQPPPV |
| | | | PKPPRVVDKLSLCIAPGKPCSSGASEITGLTTAGLEAHGRQRFNDNLATLLQVFLQR |
| | | | QPQPCCLVAHNGDRYDFPLLQAELASLSVISPLDGTFCVDSIAALKTLEQASSPSEH |
| | | | GPRKSYSLGSIYTRLYGQAPTDSHTAEGDVLALLSICQWKPQALLQWVDKHARPFST |
| | | | IKPMYGMAATTGTASPRLCAATTSSPLATANLSPSNGRSRGKRPTSPPPENVPEAPS |
| | | | REGLLAPLGLLTFLTLAIAVLYGIFLASPGQ |
| AAH63664.1 | Human DNA2 | 150 | >gi\|39793966\|gb\|AAH63664.1\| DNA2 protein [Homo sapiens] |
| | | | FAIPASRMEQLNELELLMEKSFWEEAELPAELFQKKVVASFPRTVLSTGMDNRYLvL |
| | | | AVNTVQNKEGNCEKRLVITASQSLENKELCILRNDWCSVPVEPGDIIHLEGDCTSDT |
| | | | WIIDKDFGYLILYPDMLISGTSIASSIRCMRRAVLSETFRSSDPATRQMLIGTVLHE |
| | | | VFQKAINNSFAPEKLQELAFQTIQEIRHLKEMYRLNLSQDEIKQEVEDYLPSFCKWA |
| | | | GDFMHKNTSTDFPQMQLSLPSDNSKDNSTCNIEVVKPMDIEESIWSPRFGLKGKIDV |
| | | | TVGVKIHRGYKTKYKIMPLELKTGKESNSIEHRSQVVLYTLLSQERRADPEAGLLLY |
| | | | LKTGQMYPVPANHLDKRELLKLRNQMAFSLFHRISKSATRQKTQLASLPQIIEEEKT |
| | | | CKYCSQIGNCALYSRAVEQQMDCSSVPIVMLPKIEEETQHLKQTHLEYFSLWCLMLT |
| | | | LESQSKDNKKNHQNIWLMPASEMEKSGSCIGNLIRMEHVKIVCDGQYLHNFQCKHGA |
| | | | IPVTNLMAGDRVIVSGEERSLFALSRGYVKEINMTTVTCLLDRNLSVLPESTLFRLD |
| | | | QEEKNCDIDTPLGNLSKLMENTFVSKKLRDLIIDFREPQFISYLSSVLPHDAKDTVA |
| | | | CILKGLNKPQRQAMKKVLLSKDYTLIVGMPGTGKTTTICTLVPAPEQVEKGGVSNVT |
| | | | EAKLIVFLTSIFVKAGCSPSDIGIIAPYRQQLKIINDLLARSIGMVEVNTVDKYQGR |
| | | | DKSIVLVSFVRSNKDGTVGELLKDWRRLNVAITRAKHKLILLGCVPSLNCYPPLEKL |
| | | | LNHLNSEKLIIDLPSREHESLCHILGDFQRE |
| P38859.1 | Yeast DNA2 | 151 | >gi\|731738\|sp\|P38859.1\|DNA2_YEAST RecName: Full = DNA |
| | (DNA2_YEAST) | | replication ATP-dependent helicase DNA2 |
| | | | MPGTPQKNKRSASISVSPAKKTEEKEIIQNDSKAILSKQTKRKKKYAFAPINNLNGK |
| | | | NTKVSNASVLKSIAVSQVRNTSRTKDINKAVSKSVKQLPNSQVKPKREMSNLSRHHD |
| | | | FTQDEDGPMEEVIWKYSPLQRDMSDKTTSAAEYSDDYEDVQNPSSTPIVPNRLKTVL |
| | | | SFTNIQVPNADVNQLIQENGNEQVRPKPAEISTRESLRNIDDILDDIEGDLTIKPTI |
| | | | TKFSDLPSSPIKAPNVEKKAEVNAEEVDKMDSTGDSNDGDDSLIDILTQKYVEKRKS |
| | | | ESQITIQGNTNQKSGAQESCGKNDNTKSRGEIEDHENVDNQAKTGNAFYENEEDSNC |
| | | | QRIKKNEKIEYNSSDEFSDDSLIELLNETQTQVEPNTIEQDLDKVEKMVSDDLRIAT |
| | | | DSTLSAYALRAKSGAPRDGVVRLVIVSLRSVELPKIGTQKILECIDGKGEQSSVVVR |
| | | | HPWVYLEFEVGDVIHIIEGKNIENKRLLSDDKNPKTQLANDNLLVLNPDVLFSATSV |
| | | | GSSVGCLRRSILQMQFQDPRGEPSLVMTLGNIVHELLQDSIKYKLSHNKISMEIIIQ |
| | | | KLDSLLETYSFSIIICNEEIQYVKELVMKEHAENILYFVNKFVSKSNYGCYTSISGT |
| | | | RRTQPISISNVIDIEENIWSPIYGLKGFLDATVEANVENNKKHIVPLEVKTGKSRSV |
| | | | SYEVQGLIYTLLLNDRYEIPIEFFLLYFTRDKNMTKFPSVLHSIKHILMSRNRMSMN |
| | | | FKHQLQEVFGQAQSRFELPPLLRDSSCDSCFIKESCMVLNKLLEDGTPEESGLVEGE |
| | | | FEILTNHLSQNLANYKEFFTKYNDLITKEESSITCVNKELFLLDGSTRESRSGRCLS |
| | | | GLVVSEVVEHEKTEGAYIYCFSRRRNDNNSQSMLSSQIAANDFVIISDEEGHFCLCQ |
| | | | GRVQFINPAKIGISVKRKLLNNRLLDKEKGVTTIQSVVESELEQSSLIATQNLVTYR |
| | | | IDKNDIQQSLSLARFNLLSLFLPAVSPGVDIVDERSKLCRKTKRSDGGNEILRSLLV |
| | | | DNRAPKFRDANDDPVIPYKLSKDTTLNLNQKEAIDKVMRAEDYALILGMPGTGKTTV |
| | | | IAEIIKILVSEGKRVLLTSYTHSAVDNILIKLRNTNISIMRLGMKHKVHPDTQKYVP |
| | | | NYASVKSYNDYLSKINSTSVVATTCLGINDILFTLNEKDFDYVILDEASQISMPVAL |
| | | | GPLRYGNRFIMVGDHYQLPPLVKNDAARLGGLEESLFKTFCEKHPESVAELTLQYRM |
| | | | CGDIVTLSNFLIYDNKLKCGNNEVFAQSLELPMPEALSRYRNESANSKQWLEDILEP |
| | | | TRKVVFLNYDNCPDIIEQSEKDNITNHGEAELTLQCVEGMLLSGVPCEDIGVMTLYR |
| | | | AQLRLLKKIFNKNVYDGLEILTADQFQGRDKKCIIISMVRRNSQLNGGALLKELRRV |
| | | | NVAMTRAKSKLIIIGSKSTIGSVPEIKSFVNLLEERNWVYTMCKD |
| | | | ALYKYKFPDRSNAIDEARKGCGKRTGAKPITSKSKFVSDKPIIKEILQEYES |
| AAA45863.1 | VP16 | 152 | >gi\|330318\|gb\|AAA45863.1\| VP16 [Human herpesvirus 2] |
| | | | MDLLVDDLFADRDGVSPPPPRPAGGPKNTPAAPPLYATGRLSQAQLMPSPPMPVPPA |
| | | | ALFNRLLDDLGFSAGPALCTMLDTWNEDLFSGFPTNADMYRECKFLSTLPSDVIDWG |
| | | | DAHVPERSPIDIRAHGDVAFPTLPATRDELPSYYEAMAQFFRGELRAREESYRTVLA |
| | | | NFCSALYRYLRASVRQLHRQAHMRGRNRDLREMLRTTIADRYYRETARLARVLFLHL |
| | | | YLFLSREILWAAYAEQMMRPDLFDGLCCDLESWRQLACLFQPLMFINGSLTV |
| | | | RGVPVEARRLRELNHIREHLNLPLVRSAAAEEPGAPLTTPPVLQGNQARSSGYFMLL |
| | | | IRAKLDSYSSVATSEGESVMREHAYSRGRTRNNYGSTIEGLLDLPDDDDAPAEAGLV |
| | | | APRMSFLSAGQRPRRLSTTAPITDVSLGDELRLDGEEVDMTPADALDDFDLEMLGDV |
| | | | ESPSPGMTHDPVSYGALDVDDFEFEQMFTDAMGIDDFGG |

### Example 3

I-CreI meganuclease (SEQ ID NO: 76) was chosen as the parent scaffold on which to fuse the catalytic domain of I-TevI (SEQ ID NO: 60). Wild-type I-TevI functions as a monomeric cleavase of the GIY-YIG family to generate a staggered double-strand break in its target DNA. Guided by biochemical and structural data, variable length constructs were designed from the N-terminal region of I-TevI that encompass the entire catalytic domain and deletion-intolerant region of its linker (SEQ ID NOs: 61 to 66). In all but one case, fragments were fused to the N-terminus of I-CreI with an intervening 5-residue polypeptide linker (-QGPSG-=SEQ ID NO: 67). The linker-less fusion construct naturally contained residues (-LGPDGRKA-=SEQ ID NO: 68) similar to those in the artificial linker. As I-CreI is a homodimer, all fusion constructs contain three catalytic centers (FIG. 4D): the natural I-CreI active site at the interface of the dimer and one I-TevI active site per monomer.

The activity of each “tri-functional” meganuclease was assessed using yeast assay previously described in International PCT Applications WO 2004/067736 and in (Epinat, Arnould et al. 2003; Chames, Epinat et al. 2005; Arnould, Chames et al. 2006; Smith, Grizot et al. 2006). All constructs were able to cleave the C1221 target DNA with an activity comparable to that of wild-type I-CreI (Table 4).

To validate the activity of the I-TevI catalytic domain independent of the I-CreI catalytic core, D20N point mutants were made to inactivate the I-CreI scaffold (SEQ ID NOs: 69 to 75). Tests in yeast assays showed no visible activity from the inactivated I-CreI (D20N) mutant protein alone (Table 4). However, cleavage activity could be observed for fusions having the I-TevI catalytic domain (Table 4).

TABLE 4
Table 4: Activity in Yeast assay for I-TevI/I-CreI fusions. The
relative activity of wild-type and fusion proteins on the two
parent protein targets (C1221 for I-CreI and Tev for I-TevI) is shown.
Maximal activity (++++) is seen with each given protein on
its native DNA target. I-CreI_N20 is an inactive variant of
the wild-type I-CreI scaffold. In all other cases, activity is
only detected on the C1221 target since DNA recognition is driven
by the I-CreI scaffold. The “N20” fusion variants illustrate
cleavage activity due to the I-TevI catalytic domain.

Relative Activity in Yeast
Assay (37° C.)

| | C1221 | I-TevI |
|---|---|---|
| Protein Construct | Target | Target |
| I-CreI | ++++ | − |
| I-TevI | − | ++++ |
| I-CreI_N20 | − | − |
| hTevCre_D01 | ++++ | − |
| hTevCre_D02 | ++++ | − |
| hTevCre_D03 | ++++ | − |
| hTevCre_D04 | ++++ | − |
| hTevCre_D05 | ++++ | − |
| hTevCre_D06 | ++++ | − |
| hTevCre_D01_N20 | ++ | − |
| hTevCre_D02_N20 | ++ | − |
| hTevCre_D03_N20 | ++ | − |
| hTevCre_D04_N20 | ++ | − |
| hTevCre_D05_N20 | − | − |
| hTevCre_D06_N20 | − | − |

Relative activity is scaled as: −, no activity detectable; +, <25% activity; ++, 25% to <50% activity; +++, 50% to <75% activity; ++++, 75% to 100% activity.

### Example 4

Protein-fusion scaffolds were designed based on a truncated form of I-CreI (SEQ ID NO: 76, I-CreI_X: SEQ ID NO: 77) and three different linker polypeptides (SEQ ID NOs: 78 to 80) fused to either the N- or C-terminus of the protein. Structure models were generated in all cases, with the goal of designing a “baseline” fusion linker that would traverse the I-CreI parent scaffold surface with little to no effect on its DNA binding or cleavage activities. For the two N-terminal fusion scaffolds, the polypeptide spanning residues 2 to 153 of I-CreI was used, with a K82A mutation to allow for linker placement. The C-terminal fusion scaffold contains residues 2 to 155 of wild-type I-CreI. For both fusion scaffold types, the “free” end of the linker (i.e. onto which a polypeptide can be linked) is designed to be proximal to the DNA, as determined from models built using the I-CreI/DNA complex structures as a starting point (PDB id: 1g9z). The two I-CreI N-terminal fusion scaffolds (I-CreI_NFS1=SEQ ID NO: 81 and I-CreI_NFS2=SEQ ID NO: 82) and the single C-terminal fusion scaffold (I-CreI_{—}CFS1: SEQ ID NO: 83) were tested in our yeast assay (see Example 3) and found to have activity similar to that of wild-type I-CreI (Table 5).

Colicin E7 is a non-specific nuclease of the HNH family able to process single- and double-stranded DNA. Guided by biochemical and structural data, the region of ColE7 that encompasses the entire catalytic domain (SEQ ID NO: 84) was selected. This ColE7 domain was fused to the N-terminus of either I-CreI_NFS1 (SEQ ID NO: 81) or I-CreI_NFS2 (SEQ ID NO: 83) to create hColE7Cre_D0101 (SEQ ID: 85) or hColE7Cre_D0102 (SEQ ID NO: 86), respectively. In addition, a C-terminal fusion construct, hCreColE7_D0101 (SEQ ID: 87), was generated using I-CreI_CFS1 (SEQ ID NO: 83). As I-CreI is a homodimer, all fusion constructs contain three catalytic centers (FIG. 4D): the natural I-CreI active site at the interface of the dimer and one ColE7 active site per monomer.

The activity of each “tri-functional” meganuclease was assessed using yeast assay as previously mentioned (see Example 3). All constructs were able to cleave the C1221 target DNA with an activity comparable to that of wild-type I-CreI (Table 4). To validate the activity of the ColE7 catalytic domain independent of the I-CreI catalytic core, D20N point mutants were made to inactivate the I-CreI scaffold (SEQ ID NOs: 88-93). Tests in our yeast assays showed no visible activity from the inactivated I-CreI (D20N) mutant proteins alone (Table 5). However, cleavage activity could be observed for fusions having the ColE7 catalytic domain (Table 5).

TABLE 5
Table 5: Activity in Yeast assay for ColE7/I-CreI fusions. The relative
activity of wild-type and fusion proteins on theC1221 target is shown.
I-CreI_X represents a truncated version of I-CreI based on the crystal
structure and was used as the foundation for the fusion scaffolds
(I-CreI_NFS1, I-CreI_NFS2 and I-CreI_CFS1). “N20”
constructs are inactive variants of the respective I-CreI-based scaffolds.
Activity is detected in all cases wherein the I-CreI scaffold is active or
when DNA catalysis is provided by the ColE7 domain.

| | Relative Activity in Yeast |
|---|---|
| | Assay (37° C.) |
| Protein Construct | C1221 Target |
| I-CreI | ++++ |
| I-CreI_X | ++++ |
| I-CreI_NFS1 | ++++ |
| I-CreI_NFS2 | ++++ |
| I-CreI_CFS1 | ++++ |
| I-CreI_NFS1_N20 | − |
| I-CreI_NFS2_N20 | − |
| I-CreI_CFS1_N20 | − |
| hColE7Cre_D0101 | ++++ |
| hColE7Cre_D0102 | ++++ |
| hCreColE7_D0101 | ++++ |
| hColE7Cre_D0101_N20 | +++ |
| hColE7Cre_D0102_N20 | +++ |
| hCreColE7_D0101_N20 | ++ |

Relative activity is scaled as: −, no activity detectable; +, <25% activity; ++, 25% to <50% activity; +++, 50% to <75% activity; ++++, 75% to 100% activity.

### Example 5

### Effect of Trex2 or TREX2 (SEQ ID NO: 145) on Meganuclease-Induced Mutagenesis

Human Trex2 protein (SEQ ID NO: 145) is known to exhibit a 3′ to 5′ exonuclease activity (Mazur and Perrino, 2001). A 236 amino acid functional version of Trex2 (SEQ ID NO: 194) has been fused to single-chain meganucleases (SC-MN) for measuring improvements on meganuclease-induced targeted mutagenesis of such chimeric rare-cutting endonucleases. Levels of mutagenesis induced by SC-MN-Trex2 have been compared to levels of mutagenesis induced by co-transfecting vectors independently expressing SC-MN and Trex2 protein in a dedicated cellular model and at endogenous loci in 293H cells.

### Example 5A

### Co-Transfection of Trex2 (SEQ ID NO: 145) with Meganucleases

A vector encoding meganuclease SC_GS (pCLS2690, SEQ ID NO: 153) was co-transfected into a cell line for monitoring mutagenic events in the presence or absence of a vector encoding Trex2 (pCLS7673, SEQ ID NO: 154). The SC_GS meganuclease is a single chain protein (SEQ ID NO: 193) derived from the fusion of two I-CreI variants. It recognizes a 22 bp DNA sequence (5′-TGCCCCAGGGTGAGAAAGTCCA-3′: GS_CHO.1 target, SEQ ID NO: 155) located in the first exon of the Cricetulus griseus glutamine synthetase gene. Different meganucleases such as SC_RAG1 (pCLS2222, SEQ ID NO: 156 i.e. the expression vector encoding SC_RAG1, SEQ ID NO: 58), SC_XPC4 (pCLS2510, SEQ ID NO: 157 i.e. the expression vector encoding SC_XPC4, SEQ ID NO: 190) and SC_CAPNS1 (pCLS6163, SEQ ID NO: 158 i.e. the expression vector encoding SC_CAPNS1, SEQ ID NO: 192) were co-transfected with or without a Trex2 expression vector (pCLS7673, SEQ ID NO: 154) to analyze the effect on meganuclease-induced mutagenesis at endogenous loci.

Material and Methods

a) Cellular Model to Monitor Meganuclease-Induced Mutagenesis

The plasmid pCLS6810 (SEQ ID NO: 159) was designed to quantify the NHEJ repair frequency induced by the SC_GS meganuclease (pCLS2690, SEQ ID NO: 153). The sequence used to measure SC_GS-induced mutagenesis is made of an ATG start codon followed by (i) 2 codons for alanine; (ii) an HA-tag sequence; (iii) the SC_GS recognition site; (iv) a stretch of glycine-serine di-residues; (v) an additional 2 codons for alanine as in (i) and finally; (vi) a GFP reporter gene lacking its ATG start codon. The GFP reporter gene is inactive due to a frame-shift introduced by the GS recognition site. The creation of a DNA double-strand break (DSB) by the SC_GS meganuclease followed by error-prone NHEJ events can lead to restoration of the GFP gene expression in frame with the ATG start codon. The final construct was introduced at the RAG1 locus in 293H cell line using the hsRAG1 Integration Matrix CMV Neo from cGPS® Custom Human Full Kit DD (Cellectis Bioresearch) following the provider's instructions. Using this kit, a stable cell line containing a single copy of the transgene at the RAG1 locus was obtained. Thus, after transfection of this cell line by SC_GS meganuclease expressing plasmid with or without a plasmid encoding Trex2 (pCLS7673, SEQ ID NO: 154), the percentage of GFP positive cells is directly correlated to the mutagenic NHEJ repair frequency induced by the transfected molecular entity/ies.

b) Transfection in a Cellular Model Monitoring Meganuclease-Induced Mutagenesis

One million of cells were seeded one day prior to transfection. Cells were co-transfected with 1 μg of SC_GS encoding vector (pCLS2690, SEQ ID NO: 153) and with 0, 2, 4, 6 or 9 μg of plasmid encoding Trex2 (pCLS7673 SEQ ID NO: 154) in 10 μg of total DNA by complementation with a pUC vector (pCLS0002, SEQ ID NO: 191) using 25 μl of lipofectamine (Invitrogen) according to the manufacturer's instructions. Four days following transfection, cells were harvested for flow cytometry analysis using Guava instrumentation. Genomic DNA was extracted from cell populations transfected with 1 μg of SC_GS expressing plasmid and 0, 4 and 9 μg of Trex2 encoding plasmid. Locus specific PCR were performed using the following primers: 5′-CCATCTCATCCCTGCGTGTCTCCGACTCAG (forward adaptor sequence)-10N-(sequences needed for PCR product identification)-GCTCTCTGGCTAACTAGAGAACCC (transgenic locus specific forward sequence)-3′ (SEQ ID NO: 160) and 5′-CCTATCCCCTGTGTGCCTTGGCAGTCTCAG-(reverse adaptor sequence)-TCGATCAGCACGGGCACGATGCC (transgenic locus specific reverse sequence) (SEQ ID NO: 161), and PCR products were sequenced by a 454 sequencing system (454 Life Sciences). Approximately 10,000 sequences were obtained per PCR product and then analyzed for the presence of site-specific insertion or deletion events.

c) Transfection on 293H Cells to Monitor Meganuclease-Induced Mutagenesis at Endogenous Loci

One million of cells were seeded one day prior to transfection. Cells were co-transfected with 3 μg of plasmid expressing SC_RAG1 or SC_XPC4 or SC_CAPNS1 (pCLS2222, SEQ ID NO: 156; pCLS2510, SEQ ID NO: 157 and pCLS6163, SEQ ID NO: 158 respectively) and with 0 or 2 μg of plasmid encoding Trex2 (pCSL7673 SEQ ID NO: 154) in 5 μg of total DNA by complementation with a pUC vector (pCLS0002 SEQ ID NO: 191) using 25 μl of lipofectamine (Invitrogen) according to the manufacturer's instructions. Locus specific PCR were performed using the following primers: 5′-CCATCTCATCCCTGCGTGTCTCCGACTCAG-(forward adaptor sequence)-10N-(sequences needed for PCR product identification)-locus specific forward sequence for RAG 1: GGCAAAGATGAATCAAAGATTCTGTCC-3′ (SEQ ID NO: 162), for XPC4:-AAGAGGCAAGAAAATGTGCAGC-3′ (SEQ ID NO: 163) and for CAPNS1-CGAGTCAGGGCGGGATTAAG-3′ (SEQ ID NO: 164) and the reverse primer 5′-CCTATCCCCTGTGTGCCTTGGCAGTCTCAG-(reverse adaptor sequence)-(endogenous locus specific reverse sequence for RAG1: -GATCTCACCCGGAACAGCTTAAATTTC-3′ (SEQ ID NO: 165), for XPC4: -GCTGGGCATATATAAGGTGCTCAA-3′ (SEQ ID NO: 166) and for CAPNS1: -CGAGACTTCACGGTTTCGCC-3′ (SEQ ID NO: 167). PCR products were sequenced by a 454 sequencing system (454 Life Sciences). Approximately 10,000 sequences were obtained per PCR product and then analyzed for the presence of site-specific insertion or deletion events.

Results

1—On Cellular Model Measuring Meganuclease-Induced Mutagenesis

The percentage of GFP+ cells, monitoring mutagenesis events induced by SC_GS meganuclease in a dedicated cellular model, was analyzed 96 h after a transfection with SC_GS expressing plasmid (pCLS2690 SEQ ID NO: 153) alone or with an increasing dose of Trex2 encoding vector (pCLS7673 SEQ ID NO: 154). The percentage of GFP+ cells increased with the amount of Trex2 expressing plasmid transfected. In absence of Trex2, SC_GS expression led to 0.3% of GFP+ cells whereas 2, 4, 6 and 9 μg of Trex2 encoding plasmid led to 1.3, 2.8, 3.4 and 4.8% of GFP+ respectively (FIG. 5A). This phenotypic stimulation of GFP+ cells was confirmed at a molecular level. SC_GS led to 2.4% of targeted mutagenesis whereas co-transfection of SC_GS expressing plasmid with 4 and 9 μg of Trex2 encoding vector stimulate this mutagenic DSB repair to 9.4 and 13.1% respectively (FIG. 5B). Moreover the nature of the mutagenic events was analyzed. In presence of Trex2, up to 65% of the mutagenic events correspond to the complete or partial loss of the 3′ overhang (deletion2, deletion3 and deletion4) generated by SC_GS meganuclease. In contrast, in absence of Trex2 activity, such mutagenic events are found in 20% of the total mutagenic events (FIG. 5C).

2—At Endogenous Loci

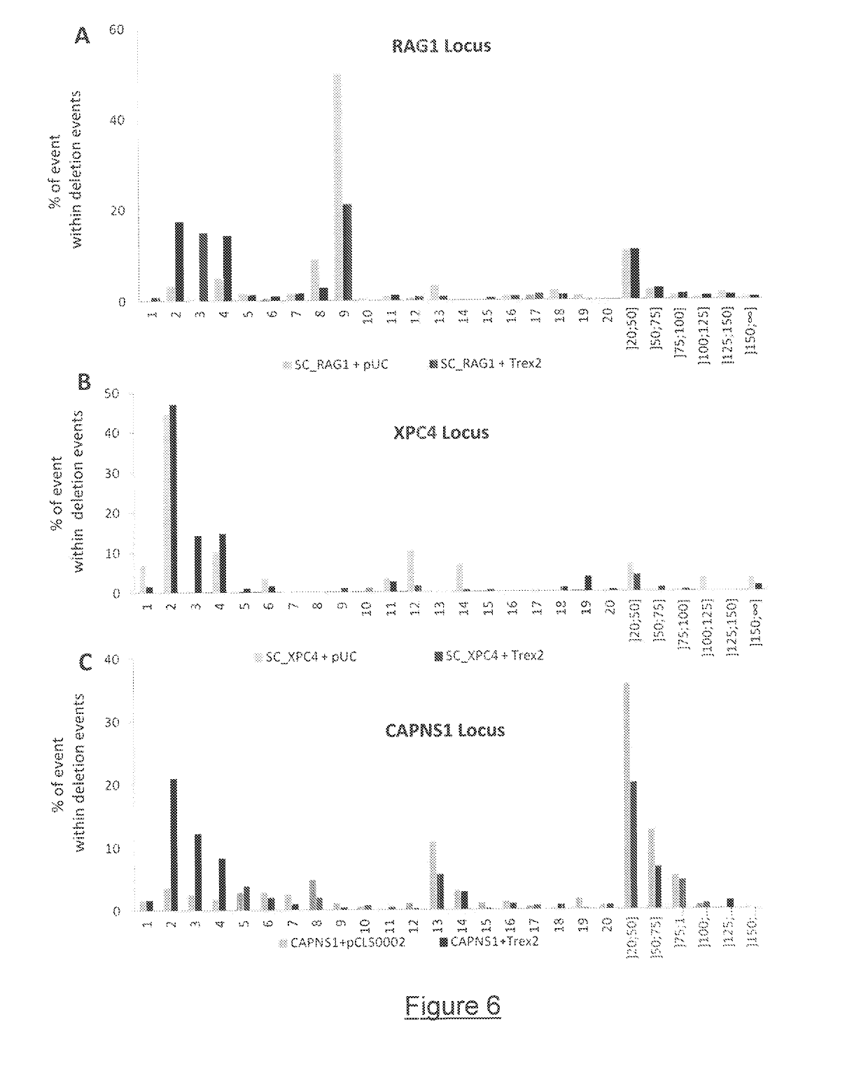

Trex2 effect on mutagenesis induced by engineered meganucleases was measured at RAG1, XPC4 and CAPNS1 endogenous loci by co-transfecting plasmids expressing SC_RAG1 or SC_XPC4 XPC4 or SC_CAPNS1 with or without Trex2 encoding plasmid. Transfections of 3 μg of meganuclease expressing vector with 2 μg of Trex2 (3/2 ratio) encoding plasmid were performed. The mutagenesis induced by the different meganucleases was quantified and analyzed three days post transfection. In these conditions, Trex2 stimulates mutagenesis at all loci studied with a stimulating factor varying from 1.4 up to 5 depending on the locus (Table 6). The nature of mutagenic events was also analyzed. It showed a modification of the pattern of the deletions induced by the meganucleases. As showed in FIG. 6, particularly at RAG1 (panelA) and CAPNS1 loci (panelC), the frequency of small deletions corresponding to degradation of 3′ overhangs is significantly increased in the presence of Trex2.

TABLE 6
Specific meganuclease-induced NHEJ quantification at endogenous
loci with or without Trex2 and corresponding stimulation factors

| | 2 μg pUC | 2 μg Trex2 | Stimulation by Trex2 |
|---|---|---|---|
| XPC4 | 0.69 | 3.41 | 4.94 |
| RAG1 | 1.88 | 5.18 | 2.75 |
| CAPNS1 | 11.28 | 16.24 | 1.44 |

### Example 5B

### Fusion of the Human Trex2 Protein to the N- or C-Terminus of an Engineered Meganuclease

Expressing Trex2 within a cell can lead to exonuclease activity at loci not targeted by the meganuclease. Moreover, for obvious reasons, co-tranfection of two expressing vectors makes difficult to control the optimum expression of both proteins. In order to bypass those difficulties and to target Trex2 activity to the DSB induced by the meganuclease, the human Trex2 protein was fused to the N- or C-terminus of the SC_GS engineered meganuclease (SEQ ID NO: 153). Four SC_GS/Trex2 fusion proteins were made and tested for their ability to cleave their target (GS_CHO.1 target). The level of mutagenesis induced by each construct was measured using the cellular model described in example 5A.

### Material and Methods

a) Making of SC_GS/Trex2 Fusion Proteins

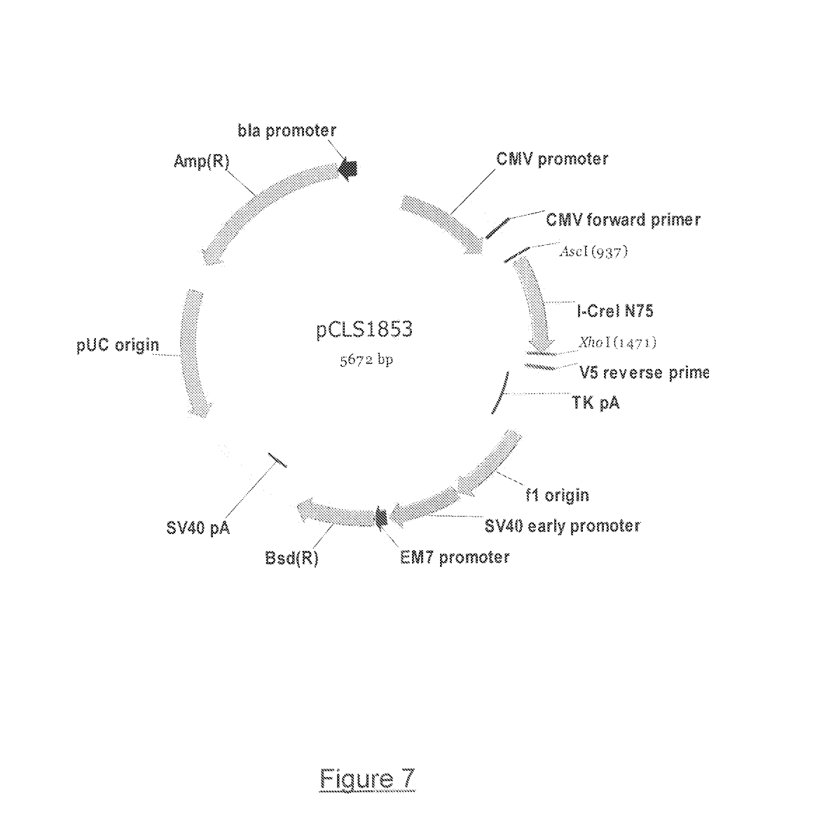

The Trex2 protein was fused to the SC_GS meganuclease either to its C-terminus or to its N-terminus using a five amino acids glycin stretch (sequence GGGGS) (SEQ ID NO: 169) or a ten amino acids glycin stretch (GGGGS)₂ (SEQ ID NO: 170) as linkers. This yielded to four protein constructs named respectively SC_GS-5-Trex, SC_GS-10-Trex, Trex-5-SC_GS, Trex-10-SC_GS (SEQ ID NO: 171 to 174). Both SC_GS and Trex2 were initially cloned into the AscI/XhoI restriction sites of the pCLS1853 (FIG. 7, SEQ ID NO: 175), a derivative of the pcDNA3.1 (Invitrogen), which drives the expression of a gene of interest under the control of the CMV promoter. The four fusion protein constructs were obtained by amplifying separately the two ORFs using a specific primer and the primer CMVfor (5′-CGCAAATGGGCGGTAGGCGT-3′; SEQ ID NO: 176) or V5reverse (5′-CGTAGAATCGAGACCGAGGAGAGG-3′; SEQ ID NO: 177), which are located on the plasmid backbone. Then, after a gel purification of the two PCR fragments, a PCR assembly was realized using the CMVfor/V5reverse oligonucleotides. The final PCR product was then digested by AscI and XhoI and ligated into the pCLS 1853 digested with these same enzymes. The following table gives the oligonucleotides that were used to create the different constructs.

TABLE 7
Oligonucleotides used to create the
different SC_GS/Trex2 constructs

| | | | SEQ | | SEQ |
|---|---|---|---|---|---|
| | Amplified | Forward | ID | Reverse | ID |
| Construct | ORF | primer | NO: | primer | NO: |
| SC_GS-5- | SC_GS | CMVfor | 176 | Link5GSRev | 179 |
| Trex | Trex2 | Link5TrexFor | 178 | V5reverse | 177 |
| SC_GS-10- | SC_GS | CMVfor | 176 | Link10GSRev | 181 |
| Trex | Trex2 | Link10TrexFor | 180 | V5reverse | 177 |
| Trex-5- | Trex2 | CMVfor | 176 | Link5TrexRev | 183 |
| SC_GS | SC_GS | Link5GSFor | 182 | V5reverse | 177 |
| Trex-10- | Trex2 | CMVfor | 176 | Link10TrexRev | 185 |
| SC_GS | SC_GS | Link10GSFor | 184 | V5reverse | 177 |

b) Extrachromosomal SSA Activity

CHO-K1 cells were transfected with the expression vector for the protein of interest and the reporter plasmid in the presence of Polyfect transfection reagent in accordance with the manufacturer's protocol (Qiagen). Culture medium was removed 72 hours after transfection and lysis/detection buffer was added for the β-galactosidase liquid assay. One liter of lysis/detection buffer contains: 100 ml of lysis buffer (10 mM Tris-HCl pH 7.5, 150 mM NaCl, 0.1% Triton X100, 0.1 mg/ml BSA, protease inhibitors), 10 ml of 100× Mg buffer (100 mM MgCl₂, 35% 2-mercaptoethanol), 110 ml of a 8 mg/ml solution of ONPG and 780 ml of 0.1M sodium phosphate pH 7.5. The OD₄₂₀ is measured after incubation at 37° C. for 2 hours. The entire process was performed using a 96-well plate format on an automated Velocityll BioCel platform (Grizot, Epinat et al. 2009).

c) Meganuclease-Induced Mutageneis

One million of cells were seeded one day prior transfection. Cells were transfected with an increasing amount (from 1 μg up to 9 μg) of plasmid encoding SC_GS (pCLS2690, SEQ ID NO: 153) or SC_GS-5-Trex (pCLS8082 SEQ ID NO: 186), SC_GS-10-Trex (pCLS8052 SEQ ID NO: 187), Trex-5-SC_GS (pCLS8053 SEQ ID NO: 188) and Trex-10-SC_GS (pCLS8054 SEQ ID NO: 189) in 10 μg of total DNA by complementation with a pUC vector (SEQ ID NO: 191) using 25 μl of lipofectamine (Invitrogen) according to the manufacturer's instructions. Three to four days following transfection, cells were harvested for flow cytometry analysis using Guava instrumentation. Cells transfected with 1 μg and 6 μg of SC_GS or SC_GS-10-Trex2 expressing plasmid were harvested for genomic DNA extraction. Locus specific PCR were performed using the following primers: 5′-CCATCTCATCCCTGCGTGTCTCCGACTCAG (forward adaptor sequence)-10N-(sequences needed for PCR product identification)-GCTCTCTGGCTAACTAGAGAACCC (transgenic locus specific forward sequence)-3′ (SEQ ID NO: 160) and 5′-CCTATCCCCTGTGTGCCTTGGCAGTCTCAG-(reverse adaptor sequence)-TCGATCAGCACGGGCACGATGCC (transgenic locus specific reverse sequence) (SEQ ID NO: 161). PCR products were sequenced by a 454 sequencing system (454 Life Sciences). Approximately 10,000 sequences were obtained per PCR product and then analyzed for the presence of site-specific insertion or deletion events.

### Results

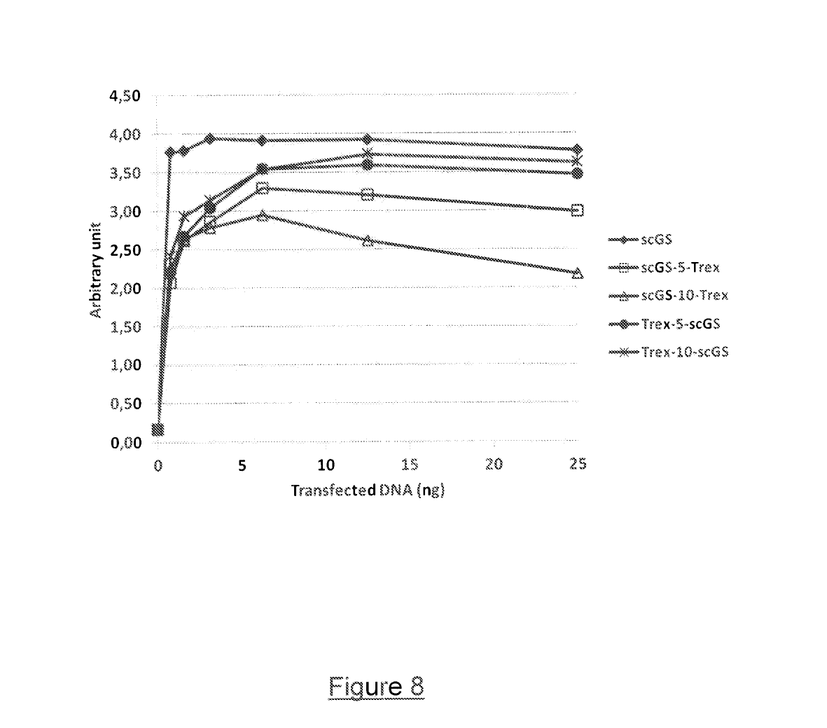

The activity of the four fusion proteins was first monitored using an extrachromosomal assay in CHO-K1 cells (Grizot, Epinat et al. 2009). The fusion of Trex2 to the SC_GS could indeed impair its folding and/or its activity. FIG. 8 shows that the four fusion proteins (SC_GS-5-Trex, SC_GS-10-Trex, Trex-5-SC_GS and Trex-10-SC_GS, SEQ ID NO: 171 to 174 encoded in plasmids of SEQ ID NO: 186 to 189) are active in that assay.

1—On Cellular Model Measuring Meganuclease-Induced Mutagenesis

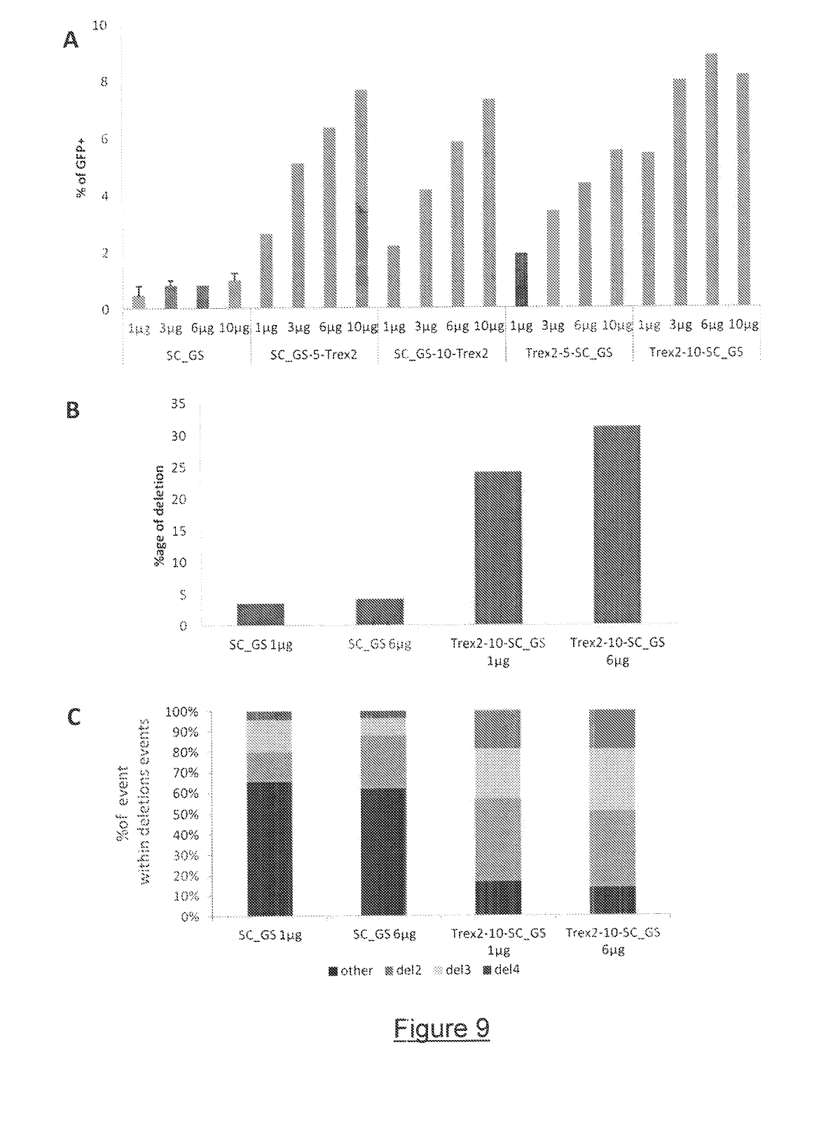

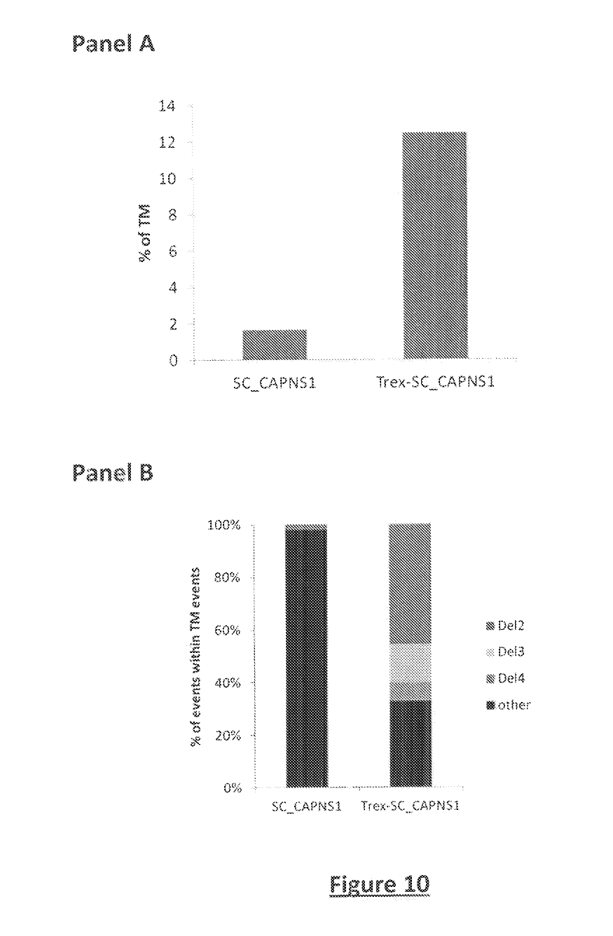

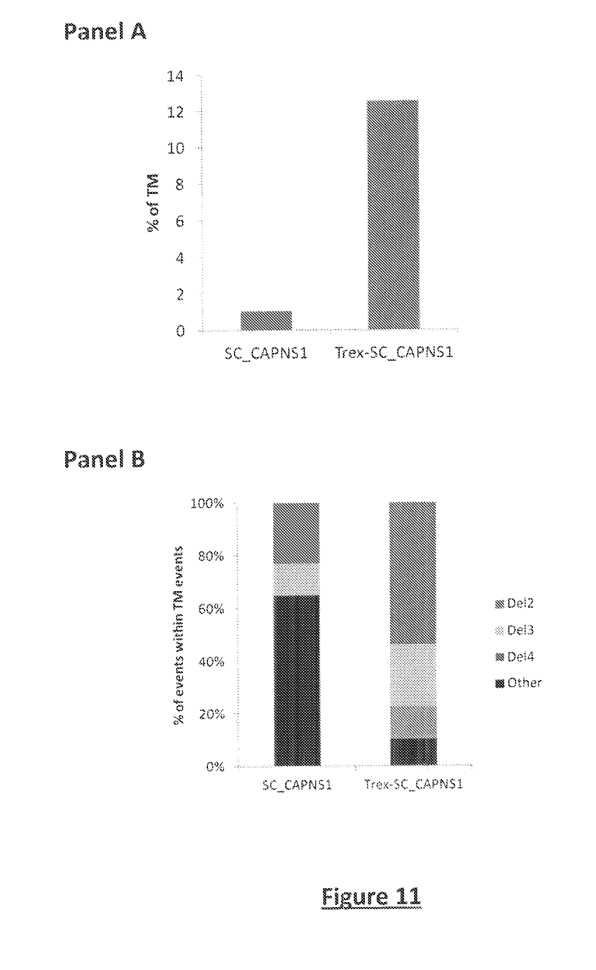

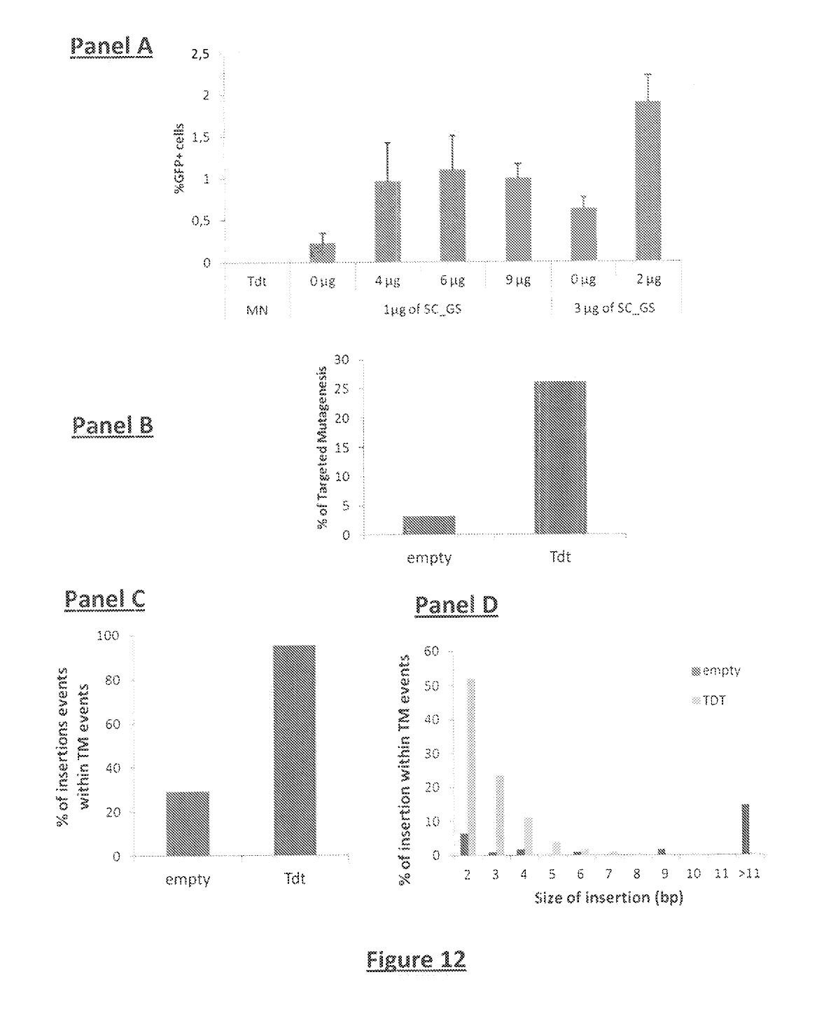

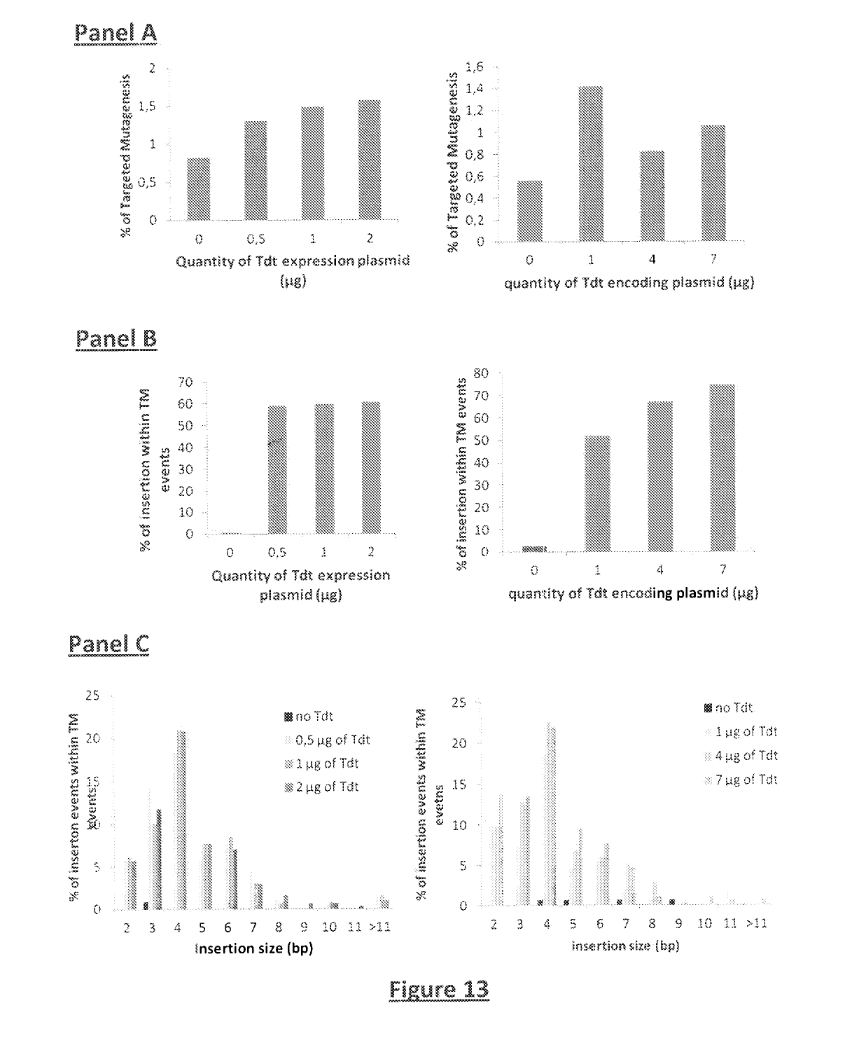

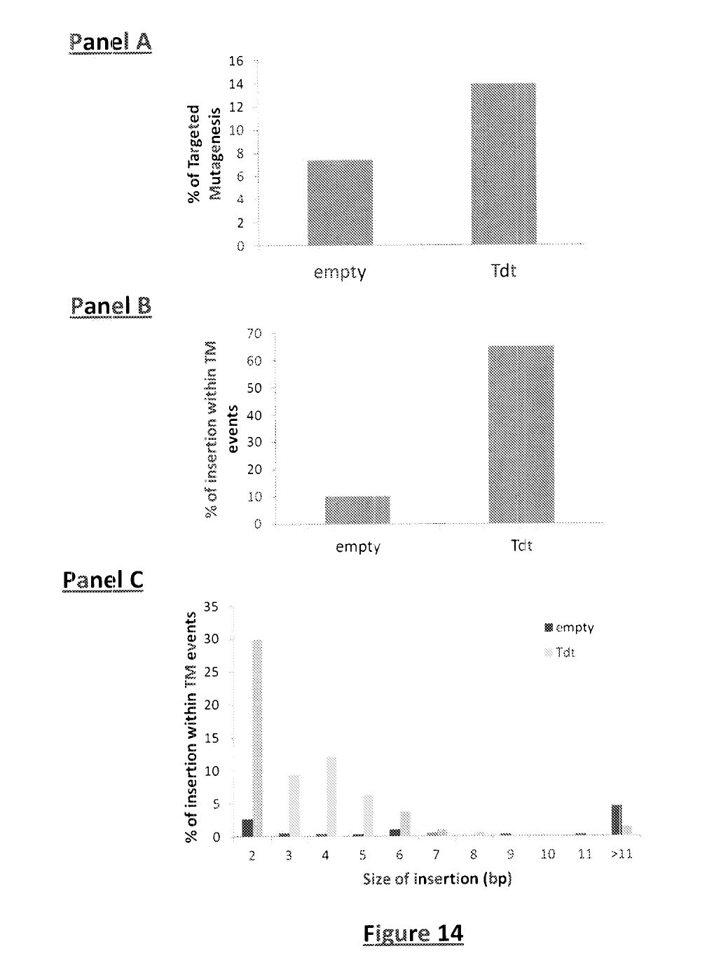

The cell line described in example 5A was transfected with plasmids expressing either SC_GS or the 4 different fusion proteins. Quantification of the percentage of GFP+ cells was determined by flow cytometry 4 days post transfection. SC_GS induced 0.5 to 1% of GFP+ cells whereas the all four fusion constructs enhance the percentage of GFP+ cells in dose dependent manner from 2 up to 9% (FIG. 9A). This strategy appears to be more efficient than the co-transfection strategy as the highest frequency of 4.5% of GFP+ cells was obtained using 9 μg of Trex2 expressing vector (FIG. 5A) whereas this frequency can be obtained using only 3 μg of any fusion expressing vector (FIG. 9A). The targeted locus was analyzed by PCR amplification followed by sequencing, after cellular transfection of 1 μg and 6 μg of SC_GS or Trex2-10-SC_GS expressing plasmid. The deletions events were greatly enhanced with the fusion construct compared to the native meganuclease. 1 μg or 6 μg of Trex2-10-SC_GS expressing plasmid led to 24% and 31% of mutagenic events all corresponding to deletions. These NHEJ frequencies were higher than the ones obtained using 4 or 9 μg of Trex2 expressing vector in co-transfection experiments (9% and 13% respectively), (FIGS. 5B and 9B). Finally molecular analysis showed that the complete or partial loss of the 3′ overhang (deletion2, deletion3 and deletion4) generated by SC_GS or the fusion Trex2-10-SC_GS were 35% and 80% respectively. Altogether these results demonstrate that the fusion protein Trex2-SC_GS is highly active as a targeted mutagenic reagent (frequency of GFP+ cells obtained, frequency of mutagenic events analyzed by deep-sequencing and the frequency of the signature of Trex2 nuclease activity).

### Example 5C

### Effect of Trex2 Fused with an Engineered Meganuclease on Mutagenesis at an Endogenous Locus in Immortalized or Primary Cell Line

Trex2 fused to SC_GS was shown to stimulate Targeted Mutagenesis [TM] at a transgenic locus in immortalized cell line. In order to apply the fusion to other engineered meganucleases and to stimulate TM in primary cell line Trex2 was fused to SC_CAPNS1 and TM was monitored at an endogenous locus in immortalized cell line as well as in primary cell line.

### Material and Methods

d) Making of Trex2-SC_CAPNS1 Fusion Protein

The Trex2 protein (SEQ ID NO: 194) was fused to the SC_CAPNS1 meganuclease

(SEQ ID NO: 192) at its N-terminus using a (GGGGS)₂ ten amino acids linker (SEQ ID NO: 170). Cloning strategy was the same as used for the fusion Trex-SC_GS. Both SC_CAPNS1 and Trex2 were initially cloned into the AscI/XhoI restriction sites of the pCLS1853 (FIG. 7, SEQ ID NO: 175), a derivative of the pcDNA3.1 (Invitrogen), which drives the expression of a gene of interest under the control of the CMV promoter. The fusion protein construct was obtained by amplifying separately the two ORFs using specific primers: for CAPNS1 Link10GSFor

(SEQ ID NO: 184)
5′-GGAGGTTCTGGAGGTGGAGGTTCCAATACCAAATATAACGAAGAGT
TC-3′

was used with V5 reverse primer 5′-CGTAGAATCGAGACCGAGGAGAGG-3′ (SEQ ID NO: 177); Trex ORF was amplified using CMVfor primer 5′-CGCAAATGGGCGGTAGGCGT-3′ (SEQ ID NO: 176) and Link10TrexRev primer

(SEQ ID NO: 185)
5′-CCTCCACCTCCAGATCCGCCACCTCCAGGAGAGGACTTTTTCTTCT
CAGA-3′.

Then, after a gel purification of the two PCR fragments, a PCR assembly was realized using the CMVfor/V5reverse oligonucleotides. The final PCR product was then digested by AscI and XhoI and ligated into the pCLS 1853 plasmid digested with these same enzymes leading to Trex-SC_CAPNS1 encoding vector (pCLS8518 of SEQ ID NO: 196 encoding Trex-SC_CAPNS1 protein of SEQ ID NO: 197).

e) Transfection on 293H Cells to Monitor Trex2-Meganuclease Fusion on Mutagenesis at an Endogenous Locus

One million of cells were seeded one day prior to transfection. Cells were transfected with 100 ng of either SC_CAPNS1 or Trex-SC_CAPNS1 encoding vector (respectively, protein sequence of SEQ ID NO: 192 encoded by pCLS6163 of SEQ ID NO: 158 and protein sequence of SEQ ID NO: 197 encoded by pCLS8518 of SEQ ID NO: 196) in 5 μg of total DNA by complementation with a pUC vector (pCLS0002 SEQ ID NO: 191) using 25 μl of lipofectamine (Invitrogen) according to the manufacturer's instructions. Three days following transfection, cells were harvested for genomic DNA extraction.

f) Transfection on Detroit Cells to Monitor Trex2-Meganuclease Fusion on Mutagenesis at an Endogenous Locus

One million of cells were seeded one day prior to transfection. Cells were co-transfected with 6 μg of either SC_CAPNS1 or Trex-SC_CAPNS1 encoding vector (respectively pCLS6163 of SEQ ID NO: 158 and pCLS8518 of SEQ ID NO: 196) in 10 μg of total DNA by complementation with a pUC vector (pCLS0002, SEQ ID NO: 191) using Amaxa (LONZA) according to the manufacturer's instructions. Three days following transfection, cells were harvested for genomic DNA extraction.

g) Deep-Sequencing at CAPNS1 Locus

PCR for deep-sequencing were performed using the following primers: 5′-CCATCTCATCCCTGCGTGTCTCCGAC-(forward adaptor sequence)-10N-(sequences needed for PCR product identification)-CGAGTCAGGGCGGGATTAAG-3′-(locus specific forward sequence) (SEQ ID NO: 199) and the reverse primer 5′-CCTATCCCCTGTGTGCCTTGGCAGTCTCAG-(reverse adaptor sequence)-CGAGACTTCACGGTTTCGCC-3′ (endogenous locus specific reverse sequence) (SEQ ID NO: 200). PCR products were sequenced by a 454 sequencing system (454 Life Sciences). Approximately 10,000 sequences were obtained per PCR product and then analyzed for the presence of site-specific insertion or deletion events.

### Results

3—In Immortalized 293H Cell Line

Wild-type 293H cells were transfected by SC_CAPNS1 or Trex-SC_CAPNS1 in order to determine if those constructs could stimulate engineered meganuclease-induced targeted mutagenesis at an endogenous locus. Transfection with SC_CAPNS1 led to 1.6% of targeted mutagenesis (TM) whereas transfection with the fusion Trex-SC_CAPNS1 stimulated TM up to 12.4% (FIG. 10, Panel A). Moreover, the analysis of the mutagenic sequences showed that the proportion of small deletions events of 2, 3 and 4 base pairs was increased from 2% of the TM events with SC_CAPNS1 to 67% with the fusion Trex-SC_CAPNS1 (FIG. 10, Panel B).

4—In Primary Detroit Cell Line

Wild type Detroit551 cells were transfected by SC_CAPNS1 or Trex-SC_CAPNS1 in order to determine if those constructs could also stimulate engineered meganuclease-induced targeted mutagenesis at an endogenous locus in primary cells. Transfection with SC_CAPNS1 led to 1.1% of TM whereas transfection with the fusion Trex-CAPNS1 stimulated TM up to 12.5% (FIG. 11, Panel A). Moreover, the analysis of the mutagenic sequences showed that the proportion of small deletions events of 2, 3 and 4 base pairs was increased from 35% of the TM events with SC_CAPNS1 to 90% with the fusion Trex-SC_CAPNS1 (FIG. 11, Panel B).

### Example 6

### Effect of Terminal Deoxynucleotidyl Transferase (Tdt) Expression on Meganuclease-Induced Mutagenesis

Homing endonucleases from the LAGLIDADG family or meganucleases recognize long DNA sequences and cleave the two DNA strands, creating a four nucleotides 3′ overhang. The cell can repair the double strand break (DSB) mainly through two mechanisms: by homologous recombination using an intact homologous template or by non homologous end joining (NHEJ). NHEJ is considered as an error prone mechanism that can induce mutations (insertion or deletion of DNA fragments) after DSB repair. Hence, after the transfection of a meganuclease into the cell, the measurement of the mutagenesis frequency at the meganuclease locus is a way to assess the meganuclease activity. Meganucleases derived from the I-CreI protein have been shown to induce mutagenesis at the genomic site, for which they have been designed (Munoz et al., 2011).

The human Tdt protein (SEQ ID NO: 201) is a 508 amino acids protein that catalyzes the addition of deoxynucleotides to the 3′-hydroxyl terminus of DNA ends. The encoded protein is expressed in a restricted population of normal and malignant pre-B and pre-T lymphocytes during early differentiation. It generates antigen receptor diversity by synthesizing non-germ line elements at DSB site after RAG1 and RAG2 endonucleases cleavage. After a meganuclease DSB induced event, such an activity could add DNA sequences at the targeted site and would thus stimulate targeted mutagenesis induced by meganuclease.

### Example 6A

### Co-Transfection of Tdt (SEQ ID NO: 201) with Meganucleases

To test this hypothesis, vector encoding meganuclease SC_GS (pCLS2690, SEQ ID NO: 153) was co-transfected on a cell line monitoring mutagenic NHEJ events in presence or absence of a vector encoding Tdt (pCLS3841 of SEQ ID NO: 202 encoding the protein of SEQ ID NO: 201). The SC_GS meganuclease (SEQ ID NO: 193) is a single chain protein where two I-CreI variants have been fused. It recognizes a 22 bp DNA sequence (5′-TGCCCCAGGGTGAGAAAGTCCA-3′: GS_CHO.1 target, SEQ ID NO: 155) located in the first exon of Cricetulus griseus glutamine synthetase gene. Moreover, two different meganucleases SC_RAG1 (pCLS2222, SEQ ID NO: 156 encoding SC_RAG1 of SEQ ID NO: 58), and SC_CAPNS1 (pCLS6163, SEQ ID NO: 158 encoding SC_CAPNS1 of SEQ ID NO: 192) were co-transfected with or without Tdt expression plasmid (pCLS3841, SEQ ID NO: 202) and the effects on meganuclease-induced mutagenesis at the endogenous loci were analyzed by deep-sequencing.

### Material and Methods

d) Cellular Model to Monitor Meganuclease-Induced Mutagenesis

The plasmid pCLS6810 (SEQ ID NO: 159) was designed to quantify NHEJ repair frequency induced by the SC_GS meganuclease (SEQ ID NO: 193). The sequence used to measure SC_GS-induced mutagenesis is made of an ATG start codon followed by i) 2 codons for alanine, ii) the tag HA sequence, iii) the SC_GS recognition site, iv) a glycine serine stretch, v) the same 2 codons for alanine as in i) and finally vi) a GFP reporter gene lacking its ATG start codon. Since by itself GFP reporter gene is inactive due to a frame-shift introduced by GS recognition sites, creation of a DNA double strand break (DSB) by SC_GS meganuclease followed by a mutagenic DSB repair event by NHEJ can lead to restoration of GFP gene expression in frame with the ATG start codon. These sequences were placed in a plasmid used to target the final construct at the RAG1 locus in 293H cell line using the hsRAG1 Integration Matrix CMV Neo from cGPS® Custom Human Full Kit DD (Cellectis Bioresearch). Using this kit, a stable cell line containing a single copy of the transgene at the RAG1 locus was obtained. Thus, after transfection of this cell line by the SC_GS meganuclease and with or without a plasmid encoding Tdt (pCLS3841, SEQ ID NO: 202), the percentage of GFP positive cells is directly correlated to the mutagenesis frequency induced by the transfected specie.

e) Transfection on Cellular Model Monitoring Meganuclease-Induced Mutagenesis

One million of cells were seeded one day prior to transfection. Cells were co-transfected either with 1 μg of SC_GS encoding vector (pCLS2690, SEQ ID NO: 153) and with 0, 4, 6 or 9 μg of plasmid encoding Tdt (pCLS3841 SEQ ID NO: 202) or with 3 μg of SC_GS encoding plasmid with 0 or 2 μg of Tdt encoding vector in 5 or 10 μg of total DNA, respectively, by complementation with a pUC vector (pCLS0002, SEQ ID NO: 191) using 25 μl of lipofectamine (Invitrogen) according to the manufacturer's instructions. Three days following transfection, cells were harvested for flow cytometry analysis using Guava instrumentation. Conditions corresponding to 3 μg of SC_GS encoding vector with 0 or 2 μg of Tdt encoding plasmid were harvested for genomic DNA extraction. PCR for deep-sequencing were performed using the following primers: 5′-CCATCTCATCCCTGCGTGTCTCCGACTCAG (forward adaptor sequence)-10N-(sequences needed for PCR product identification)-GCTCTCTGGCTAACTAGAGAACCC (transgenic locus specific forward sequence)-3′ (SEQ ID NO: 160) and 5′-CCTATCCCCTGTGTGCCTTGGCAGTCTCAG-(reverse adaptor sequence)-TCGATCAGCACGGGCACGATGCC (transgenic locus specific reverse sequence)-3′ (SEQ ID NO: 161). PCR products were sequenced by a 454 sequencing system (454 Life Sciences). Approximately 10,000 sequences were obtained per PCR product and then analyzed for the presence of site-specific insertion or deletion events.

f) Transfection on 293H Cells to Monitor Meganuclease-Induced Mutagenesis at Endogenous Loci

One million of cells were seeded one day prior to transfection. Cells were co-transfected with 3 μg of SC_RAG1 encoding vector (pCLS2222, SEQ ID NO: 156) with 0.5, 1 and 2 μg or with 1, 3 and 7 μg of plasmid encoding Tdt (pCLS3841, SEQ ID NO: 202) in, respectively, 5 or 10 μg of total DNA by complementation with a pUC vector (pCLS0002, SEQ ID NO: 191) using 25 μl of lipofectamine (Invitrogen) according to the manufacturer's instructions. Three μg of SC_CAPNS1 encoding vector (pCLS6163 SEQ ID NO: 158) were co-transfected with 2 μg of empty vector plasmid (pCLS0002, SEQ ID NO: 191) or Tdt encoding plasmid (pCSL3841, SEQ ID NO: 202) using 25 μl of lipofectamine (Invitrogen) according to the manufacturer's instructions. Seven days following transfection, cells were harvested for genomic DNA extraction. PCR for deep-sequencing were performed using the following primers: 5′-CCATCTCATCCCTGCGTGTCTCCGACTCAG-(forward adaptor sequence)-10N-(sequences needed for PCR product identification) (SEQ ID NO: 5)—locus specific forward sequence for RAG 1: GGCAAAGATGAATCAAAGATTCTGTCC-3′ (SEQ ID NO: 162) and for CAPNS1: CGAGTCAGGGCGGGATTAAG-3′(SEQ ID NO: 164) and the reverse primer 5′-CCTATCCCCTGTGTGCCTTGGCAGTCTCAG-(reverse adaptor sequence) (SEQ ID NO: 6)-(endogenous locus specific reverse sequence for RAG1: -GATCTCACCCGGAACAGCTTAAATTTC-3′ (SEQ ID NO: 165) and for CAPNS1: -CGAGACTTCACGGTTTCGCC-3′ (SEQ ID NO: 167). PCR products were sequenced by a 454 sequencing system (454 Life Sciences). Approximately 10,000 sequences were obtained per PCR product and then analyzed for the presence of site-specific insertion or deletion events.

### Results

1—On Cellular Model Measuring Meganuclease-Induced Mutagenic NHEJ Repair

A cell line measuring mutagenic NHEJ repair induced by SC_GS was created. The percentage of GFP+ cells, monitoring the mutagenic NHEJ repair, was analyzed 96 h after a transfection with SC_GS (pCLS2690, SEQ ID NO: 153) alone or with an increasing dose of Tdt encoding vector (pCLS3841, SEQ ID NO: 202). Without the presence of Tdt, SC_GS transfection led to 0.2+/−0.1% of GFP+ cells whereas all doses of Tdt encoding plasmid led to 1.0+/−0.4% of GFP+ cells (FIG. 12, panel A). Transfection with 3 μg of SC_GS encoding plasmid with pUC vector led to 0.6+/−0.1% of GFP+ cells while in presence of 2 μg of Tdt encoding plasmid the percentage of GFP+ cells was stimulated to 1.9+/−0.3% of GFP+ cells. Conditions corresponding to 3 μg of SC_GS with 2 μg of empty or Tdt encoding vector were analyzed by deep-sequencing. Transfection with SC_GS and an empty vector led to 3.2% of Targeted Mutagenesis (TM) while in presence of Tdt expressing plasmid, TM was stimulated up to 26.0% (FIG. 12, panel B). In absence of Tdt the insertion events represented 29% of total TM events while in presence of Tdt these insertion events were increased up to 95.3% (FIG. 12, panel C). Finally, the analysis of insertion sizes in presence of Tdt encoding plasmid led to a specific hallmark of insertion with small insertions ranging from 2 to 8 bp (FIG. 12, panel D).

2—At Endogenous RAG1 Locus

Wild type 293H cells were transfected by SC_RAG1 encoding vector (pCLS2222, SEQ ID NO: 156) with different doses of Tdt encoding plasmid (pCLS3841, SEQ ID NO: 202) in order to determine if Tdt could stimulate engineered meganuclease-induced targeted mutagenesis at an endogenous locus. Two different transfections were performed with 3 μg of SC_RAG1 encoding vector (pCLS2222, SEQ ID NO: 156) with either 0.5, 1 and 2 μg or 1, 3 and 7 μg of plasmid expressing Tdt (pCLS3841, SEQ ID NO: 202) in 5 or 10 μg of total DNA by complementation with an empty vector (pCLS0002, SEQ ID NO: 191) respectively. In absence of Tdt expressing vector, the targeted mutagenesis (TM) varies between 0.5 and 0.8%. When Tdt was present TM was stimulated up to 1.6% (FIG. 13, panel A). The nature of mutagenic DSB repair was analyzed and showed a modification of the pattern of the TM events induced by the meganuclease. As showed in FIG. 13, panel B, the percentage of insertion was almost null in absence of Tdt whereas in presence of Tdt expressing vector this percentage represents 50 up to 70% of the TM events. The sizes of insertions were also analyzed and in presence of Tdt a specific pattern of insertions appeared corresponding to small insertions ranging from 2 to 8 bp (FIG. 13, panel C). Finally the sequences of these insertions seem to show that they are apparently random (Table 8).

TABLE 8
Example of sequences with insertion at RAG1 endogenous locus in presence of Tdt.

| Sequences with insertion | Insertion size | Insertion position |
|---|---|---|
| attgttctcaggcgtacctcagccagc | 2 | 5′ |
| attgttctcaggtacatctcagccagc | 2 | 3′ |
| attgttctcaggtacccctcagccagc | 2 | 3′ |
| attgttctcaggtacgggctcagccagc | 3 | 3′ |
| attgttctcagggcgtacctcagccagc | 3 | 5′ |
| attgttctcaggtacagtctcagccagc | 3 | 3′ |
| attgttctcaggtacggggctcagccag | 4 | 3′ |
| attgttctcagacccgtacctcagccagc | 4 | 5′ |
| attgttctcagcctcgtacctcagccagc | 4 | 5′ |
| attgttctcagcttcgtacctcagccagc | 4 | 5′ |
| attgttctcaggtactggactcagccagc | 4 | 3′ |
| attgttctcaggtacagggctcagccagc | 4 | 3′ |
| attgttctcaggtacgggaactcagccagc | 5 | 3′ |
| attgttctcaggtacgaaggctcagccagc | 5 | 3′ |
| attgttctcagttcctgtacctcagccagc | 5 | 5′ |
| attgttctcaggtacgggtggctcagccagc | 6 | 3′ |
| attgttctcaggtactggttactcagccagc | 6 | 3′ |
| attgttctcaggtacccatacctcagccagc | 6 | 3′ |
| attgttctcaggttacctgtacctcagccagc | 7 | 5′ |
| attgttctcaggtacaagggggctcagccagc | 7 | 3′ |
| attgttctcagggccgcccgtacctcagccagc | 8 | 5′ |

3—At Endogenous CAPNS1 Locus

Wild type 293H cells were transfected with 3 μg of plasmid encoding SC_CAPNS1 meganuclease (pCLS6163, SEQ ID NO: 158) with 0 or 2 μg of Tdt encoding plasmid (pCLS3841, SEQ ID NO: 202) (in 5 μg of total DNA) in order to determine Tdt expression effect at another endogenous locus. In absence of Tdt expressing vector, the targeted mutagenesis (TM) was 7.4%. When Tdt was present TM was stimulated up to 13.9% (FIG. 14, panel A). The nature of mutagenic DSB repair was analyzed and showed a modification of the pattern of the TM events induced by the meganuclease. As showed in FIG. 14, panel B, insertion events represented 10% of total TM events in absence of Tdt whereas in presence of Tdt expressing vector insertion events represented 65% of the TM events. The sizes of insertions were also analyzed and in presence of Tdt a specific pattern of insertions appeared corresponding to small insertions ranging from 2 to 6 bp. Finally, the sequence analysis of these insertions seems to show that they are apparently random (Table 9).

TABLE 9
Example of sequences with insertion at CAPNS1 endogenous locus in presence of
Tdt.

| Sequences with insertion | Insertion size | Insertion position |
|---|---|---|
| cagggccgcggtgcgcagtgtccgac | 2 | 3′ |
| cagggccgcgccgtgcagtgtccgac | 2 | 5′ |
| cagggccgcggcgtgcagtgtccgac | 2 | 5′ |
| cagggccgcggtgcacagtgtccgac | 2 | 3′ |
| cagggccgcggccgtgcagtgtccgac | 3 | 5′ |
| cagggccgcggtgctgcagtgtccgac | 3 | 3′ |
| cagggccgcgcctgtgcagtgtccgac | 3 | 5′ |
| cagggccgcgttctgtgcagtgtccgac | 4 | 5′ |
| cagggccgcggtgcgggcagtgtccgac | 4 | 3′ |
| cagggccgcggtccgtgcagtgtccgac | 4 | 5′ |
| cagggccgcggtgcaggcagtgtccgac | 4 | 3′ |
| cagggccgcggtgcaaagcagtgtccgac | 5 | 3′ |
| cagggccgcggtgcagtgcagtgtccgac | 5 | 5′ |
| cagggccgcggtgcggtgcagtgtccgac | 6 | 5′ |
| cagggccgcgtgtctgtgcagtgtccgac | 5 | 5′ |
| cagggccgcggtgcaaggtcagtgtccgac | 6 | 3′ |
| cagggccgcggtgcccgtgcagtgtccgac | 6 | 5′ |
| cagggccgcggtgcaagtgcagtgtccgac | 6 | 5′ |
| cagggccgcggtgcaagcagggagtgtccgac | 8 | 3′ |

### Example 6B

### Fusion of the Human Tdt to Meganucleases: Effect on Targeted Mutagenesis

Co-transfection of Tdt (SEQ ID NO: 201) with meganuclease encoding plasmids was shown to increase the rate of mutagenesis induced by meganucleases. However, this strategy implies the presence of two plasmids within the cell at the same time. Moreover it would be of benefit to target the Tdt activity at the newly created DSB upon Meganuclease's cleavage. Thus, a chimeric protein comprising TdT and Meganuclease proteins is engineered. The human Tdt protein (SEQ ID NO: 201) is fused to the N- or C-terminus of different Single chain engineered meganucleases SC_MN such as SC_GS (SEQ ID NO: 193), SC_RAG (SEQ ID NO: 58) and SC_CAPNS1 (SEQ ID NO: 192). Two SC_MN fused to Tdt protein are made: either at the N terminal domain or C terminal domain of the considered meganuclease. Those constructed are tested for their ability to increase mutagenic activity at the locus of interest.

### Material and Methods

h) Making of SC_MN/Tdt Fusion Proteins

The Tdt protein is fused to the SC_MN meganuclease either to its C-terminus or to its N-terminus using a ten amino acids linker (GGGGS)₂ (SEQ ID NO: 170). This yields to two protein constructs named respectively SC_MN-Tdt or Tdt-SC_MN. All SC_MN were initially cloned into the AscI/XhoI restriction sites of the pCLS 1853 (FIG. 7, SEQ ID NO: 175), a derivative of the pcDNA3.1 (Invitrogen), which drives the expression of a gene of interest under the control of the CMV promoter. The two fusion proteins for each SC_MN/Tdt constructs are obtained by amplifying separately the two ORFs using specific primers. The following table 10 gives the oligonucleotidic sequences that are used to create the different SC_GS/Tdt constructs.

TABLE 10
Oligonucleotides to create different SC_GS/Tdt constructs

| | | | SEQ | | SEQ |
|---|---|---|---|---|---|
| | Amplified | Forward | ID | Reverse | ID |
| Construct | ORF | primer | NO: | primer | NO: |
| SC_MN- | SC_MN | CMVfor | 176 | Link10GSRev | 181 |
| TDT | TDT | LinkTDTFor | 203 | TDTRev | 204 |
| TDT- | SC_MN | Link10GSFor | 184 | V5rev | 177 |
| SC_MN | TDT | TDTFor | 205 | Link10TDTRev | 206 |

Then, after a gel purification of the two PCR fragments, a PCR assembly is realized using the CMVfor (SEQ ID NO: 176) and TDTRev (SEQ ID NO: 204) oligonucleotides for Cter fusion of Tdt to SC_MN or using TDTFor (SEQ ID NO: 205) and V5Rev (SEQ ID NO: 177) for Nter fusion of Tdt to SC_MN. The final PCR product is cloned in a pTOPO vector then digested by AscI and XhoI and ligated into the pCLS 1853 vector (SEQ ID NO: 175) pre-digested with these same enzymes.

### Example 7

### Impact of Co-Transfection with Two Nucleases Targeting Two Sequences Separated by 173 Base Pairs (bp) on Mutagenesis Frequency

To investigate the impact on mutagenesis frequency induced by two nucleases targeting two nearby sites, co-transfection with two engineered nucleases targeting DNA sequences within the RAG1 gene was performed. Nucleases consist of an engineered meganuclease (N1) (SC_RAG of SEQ ID NO: 216) encoded by pCLS2222 (SEQ ID NO: 156) cleaving the DNA sequence 5′-TTGTTCTCAGGTACCTCAGCCAGC-3′ (T1) (SEQ ID NO: 207) and a TALEN (N2) [SEQ ID NO: 209-210 respectively encoded by pCLS8964 (SEQ ID NO: 211) and pCLS8965 (SEQ ID NO: 212)] targeting DNA sequence 5′-TATATTTAAGCACTTATATGTGTGTAACAGGTATAAGTAACCATAAACA-3′ (T2) (SEQ ID NO: 208). These two recognition sites are separated by 173 bp.

### Material and Methods

Cells Transfection

The human 293H cells (ATCC) were plated at a density of 1.2×10⁶ cells per 10 cm dish in complete medium [DMEM supplemented with 2 mM L-glutamine, penicillin (100 IU/ml), streptomycin (100 μg/ml), amphotericin B (Fongizone: 0.25 μg/ml, Invitrogen-Life Science) and 10% FBS]. The next day, cells were transfected with 10 μg of total DNA containing both nucleases expressing plasmids (3 μg of N1 and 0.25 μg of each monomer of N2), with Lipofectamine 2000 transfection reagent (Invitrogen) according to the manufacturer's protocol. As control, each nuclease was expressed alone. For all conditions, samples were completed at 10 μg of total DNA with an empty vector pCLS0003 (SEQ ID NO: 213).

Two days after, cells were collected and genomic extraction was performed. The mutagenesis frequency was determined by Deep sequencing. The T1 and T2 targets were amplified with specific primers flanked by specific adaptator needed for High Throughput Sequencing on the 454 sequencing system (454 Life Sciences)

At T1 and T2 loci, primers F_T2:
(SEQ ID NO: 214)
5′CCATCTCATCCCTGCGTGTCTCCGACTCAGTAGCTTTACATTTACTGA
ACAAATAAC-3′
and
R_T1:
(SEQ ID NO: 215)
5′CCTATCCCCTGTGTGCCTTGGCAGTCTCAGGATCTCACCCGGAACAGC
TTAAATTTC-3′

were used. 5,000 to 10,000 sequences per sample were analyzed.

### Results

The rate of mutations induced by the nucleases N1 and N2 at the targets T1 and T2 was measured by deep sequencing. Results are presented in Table 11. 0.63% of PCR fragments carried a mutation in samples corresponding to cells transfected with the N1 nuclease. Similarly, 1.46% of PCR fragments carried a mutation in sample corresponding to cells transfected with the N2 nuclease. The rate of induced mutagenesis increased up to 1.33% on T1 target and up to 2.48% on T2 target when the cells were transfected with plasmids expressing both N1 and N2, showing that the presence of two nucleases targeting two nearby sequences stimulates up to about two folds the frequency of mutagenesis. Interestingly, within the samples transfected with only one nuclease plasmid, the majority of deletions observed are small deletions. In contrast, within the sample co-transfected with both nucleases expressing plasmids a large fraction of deletions are large deletions (>197 bp), corresponding to the intervening sequences between the two cleavage sites.

Thus, it was observed that co-transfection of two nucleases targeting two nearby sequences separated by 173 bp stimulates the mutagenesis frequency.

TABLE 11
Mutagenesis rate induction by two nucleases
targeting two nearby sequences

| Nucleases | % of Mutagenesis at T1 target | % of Mutagenesis at T2 target |
|---|---|---|
| N1 | 0.63 | 0 |
| N2 | 0 | 1.46 |
| N1 + N2 | 1.33 | 2.48 |

### LIST OF CITED REFERENCES

Arimondo, P. B., C. J. Thomas, et al. (2006). “Exploring the cellular activity of camptothecin-triple-helix-forming oligonucleotide conjugates.” Mol Cell Biol 26(1): 324-33.
Arnould, S., P. Chames, et al. (2006). “Engineering of large numbers of highly specific homing endonucleases that induce recombination on novel DNA targets.” J Mol Biol 355(3): 443-58.
Arnould, S., C. Perez, et al. (2007). “Engineered I-CreI derivatives cleaving sequences from the human XPC gene can induce highly efficient gene correction in mammalian cells.” J Mol Biol 371(1): 49-65.
Ashworth, J., J. J. Havranek, et al. (2006). “Computational redesign of endonuclease DNA binding and cleavage specificity.” Nature 441(7093): 656-9.
Beumer, K. J., J. K. Trautman, et al. (2008). “Efficient gene targeting in Drosophila by direct embryo injection with zinc-finger nucleases.” Proc Natl Acad Sci USA 105(50): 19821-6.
Boch, J., H. Scholze, et al. (2009). “Breaking the code of DNA binding specificity of TAL-type III effectors.” Science 326(5959): 1509-12.
Bolduc, J. M., P. C. Spiegel, et al. (2003). “Structural and biochemical analyses of DNA and RNA binding by a bifunctional homing endonuclease and group I intron splicing factor.” Genes Dev 17(23): 2875-88.
Britt, A. B. (1999). “Molecular genetics of DNA repair in higher plants.” Trends Plant Sci 4(1): 20-25.
Burden and O. N. (1998). “Mechanism of action of eukaryotic topoisomerase II and drugs targeted to the enzyme.” Biochim Biophys Acta. 1400(1-3): 139-154.
Capecchi, M. R. (1989). “The new mouse genetics: altering the genome by gene targeting.” Trends Genet. 5(3): 70-6.
Cathomen, T. and J. K. Joung (2008). “Zinc-finger nucleases: the next generation emerges.” Mol Ther 16(7): 1200-7.
Chames, P., J. C. Epinat, et al. (2005). “In vivo selection of engineered homing endonucleases using double-strand break induced homologous recombination.” Nucleic Acids Res 33(20): e178.
Chevalier, B., M. Turmel, et al. (2003). “Flexible DNA target site recognition by divergent homing endonuclease isoschizomers I-CreI and I-MsoI.” J Mol Biol 329(2): 253-69.
Chevalier, B. S., T. Kortemme, et al. (2002). “Design, activity, and structure of a highly specific artificial endonuclease.” Mol Cell 10(4): 895-905.
Chevalier, B. S., R. J. Monnat, Jr., et al. (2001). “The homing endonuclease I-CreI uses three metals, one of which is shared between the two active sites.” Nat Struct Biol 8(4): 312-6.
Chevalier, B. S, and B. L. Stoddard (2001). “Homing endonucleases: structural and functional insight into the catalysts of intron/intein mobility.” Nucleic Acids Res 29(18): 3757-74.
Choulika, A., A. Perrin, et al. (1995). “Induction of homologous recombination in mammalian chromosomes by using the I-SceI system of Saccharomyces cerevisiae.” Mol Cell Biol 15(4): 1968-73.
Christian, M., T. Cermak, et al. (2010). “Targeting DNA double-strand breaks with TAL effector nucleases.” Genetics 186(2): 757-61.
Cohen-Tannoudji, M., S. Robine, et al. (1998). “I-SceI-induced gene replacement at a natural locus in embryonic stem cells.” Mol Cell Biol 18(3): 1444-8.
Critchlow, S. E. and S. P. Jackson (1998). “DNA end-joining: from yeast to man.” Trends Biochem Sci 23(10): 394-8.
Donoho, G., M. Jasin, et al. (1998). “Analysis of gene targeting and intrachromosomal homologous recombination stimulated by genomic double-strand breaks in mouse embryonic stem cells.”Mol Cell Biol 18(7): 4070-8.
Doyon, J. B., V. Pattanayak, et al. (2006). “Directed evolution and substrate specificity profile of homing endonuclease I-SceI.” J Am Chem Soc 128(7): 2477-84.
Doyon, Y., J. M. McCammon, et al. (2008). “Heritable targeted gene disruption in zebrafish using designed zinc-finger nucleases.” Nat Biotechnol 26(6): 702-8.
Dujon, B., L. Colleaux, et al. (1986). “Mitochondrial introns as mobile genetic elements: the role of intron-encoded proteins.” Basic Life Sci 40: 5-27.
Eisenschmidt, K., T. Lanio, et al. (2005). “Developing a programmed restriction endonuclease for highly specific DNA cleavage.” Nucleic Acids Res 33(22): 7039-47.
Endo, M., K. Osakabe, et al. (2006). “Molecular characterization of true and ectopic gene targeting events at the acetolactate synthase gene in Arabidopsis.” Plant Cell Physiol 47(3): 372-9.
Endo, M., K. Osakabe, et al. (2007). “Molecular breeding of a novel herbicide-tolerant rice by gene targeting.” Plant J 52(1): 157-66.
Epinat, J. C., S. Arnould, et al. (2003). “A novel engineered meganuclease induces homologous recombination in yeast and mammalian cells.” Nucleic Acids Res 31(11): 2952-62.
Feldmann, E., V. Schmiemann, et al. (2000). “DNA double-strand break repair in cell-free extracts from Ku80-deficient cells: implications for Ku serving as an alignment factor in non-homologous DNA end joining.” Nucleic Acids Res 28(13): 2585-96.
Gimble, F. S., C. M. Moure, et al. (2003). “Assessing the plasticity of DNA target site recognition of the PI-SceI homing endonuclease using a bacterial two-hybrid selection system.” J Mol Biol 334(5): 993-1008.
Gouble, A., J. Smith, et al. (2006). “Efficient in toto targeted recombination in mouse liver by meganuclease-induced double-strand break.” J Gene Med 8(5): 616-22.
Grizot, S., J. Smith, et al. (2009). “Efficient targeting of a SCID gene by an engineered single-chain homing endonuclease.” Nucleic Acids Res 37(16): 5405-19.
Haber, J. (2000). “Partners and pathwaysrepairing a double-strand break.” Trends Genet. 16(6): 259-264.
Haber, J. E. (1995). “In vivo biochemistry: physical monitoring of recombination induced by site-specific endonucleases.” Bioessays 17(7): 609-20.
Hanin, M., S. Volrath, et al. (2001). “Gene targeting in Arabidopsis.” Plant J 28(6): 671-7.
Ichiyanagi, K., Y. Ishino, et al. (2000). “Crystal structure of an archaeal intein-encoded homing endonuclease PI-PfuI.” J Mol Biol 300(4): 889-901.
Kalish, J. M. and P. M. Glazer (2005). “Targeted genome modification via triple helix formation.”Ann N Y Acad Sci 1058: 151-61.
Kim, Y. G., J. Cha, et al. (1996). “Hybrid restriction enzymes: zinc finger fusions to Fok I cleavage domain.” Proc Natl Acad Sci USA 93(3): 1156-60.
Kirik, A., S. Salomon, et al. (2000). “Species-specific double-strand break repair and genome evolution in plants.” Embo J 19(20): 5562-6.
Li, H., H. Vogel, et al. (2007). “Deletion of Ku70, Ku80, or both causes early aging without substantially increased cancer.” Mol Cell Biol 27(23): 8205-14.
Li, T., S. Huang, et al. (2010). “TAL nucleases (TALNs): hybrid proteins composed of TAL effectors and FokI DNA-cleavage domain.” Nucleic Acids Res 39(1): 359-72.
Lieber, M. R. and Z. E. Karanjawala (2004). “Ageing, repetitive genomes and DNA damage.” Nat Rev Mol Cell Biol. 5(1): 69-75.
Lloyd, A., C. L. Plaisier, et al. (2005). “Targeted mutagenesis using zinc-finger nucleases in Arabidopsis.” Proc Natl Acad Sci USA 102(6): 2232-7.
Ma, J., E. Kim, et al. (2003). “Yeast Mre11 and Rad1 proteins define a Ku-independent mechanism to repair double-strand breaks lacking overlapping end sequences.” Mol Cell Biol. 23(23): 8820-8828.
Meng, X., M. B. Noyes, et al. (2008). “Targeted gene inactivation in zebrafish using engineered zinc-finger nucleases.” Nat Biotechnol 26(6): 695-701.
Moore, I., M. Samalova, et al. (2006). “Transactivated and chemically inducible gene expression in plants.” Plant J 45(4): 651-83.
Moore, J. K. and J. E. Haber (1996). “Cell cycle and genetic requirements of two pathways of nonhomologous end-joining repair of double-strand breaks in Saccharomyces cerevisiae.” Mol Cell Biol 16(5): 2164-73.
Moscou, M. J. and A. J. Bogdanove (2009). “A simple cipher governs DNA recognition by TAL effectors.” Science 326(5959): 1501.
Moure, C. M., F. S. Gimble, et al. (2002). “Crystal structure of the intein homing endonuclease PI-SceI bound to its recognition sequence.” Nat Struct Biol 9(10): 764-70.
Moure, C. M., F. S. Gimble, et al. (2003). “The crystal structure of the gene targeting homing endonuclease I-SceI reveals the origins of its target site specificity.” J Mol Biol 334(4): 685-95.
Nagy, Z. and E. Soutoglou (2009). “DNA repair: easy to visualize, difficult to elucidate.” Trends Cell Biol 19(11): 617-29.
Nouspikel, T. (2009). “DNA repair in mammalian cells: Nucleotide excision repair: variations on versatility.” Cell Mol Life Sci 66(6): 994-1009.
Padidam, M. (2003). “Chemically regulated gene expression in plants.” Curr Opin Plant Biol 6(2): 169-77.
Paques, F. and P. Duchateau (2007). “Meganucleases and DNA double-strand break-induced recombination: perspectives for gene therapy.” Curr Gene Ther 7(1): 49-66.
Paques, F. and J. E. Haber (1999). “Multiple pathways of recombination induced by double-strand breaks in Saccharomyces cerevisiae.” Microbiol Mol Biol Rev 63(2): 349-404.
Pingoud, A. and G. H. Silva (2007). “Precision genome surgery.” Nat Biotechnol 25(7): 743-4.
Porteus, M. H. and D. Carroll (2005). “Gene targeting using zinc finger nucleases.” Nat Biotechnol 23(8): 967-73.
Posfai, G., V. Kolisnychenko, et al. (1999). “Markerless gene replacement in Escherichia coli stimulated by a double-strand break in the chromosome.” Nucleic Acids Res 27(22): 4409-15.
Povirk, L. F. (1996). “DNA damage and mutagenesis by radiomimetic DNA-cleaving agents: bleomycin, neocarzinostatin and other enediynes.” Mutat Res 355(1-2): 71-89.
Puchta, H., B. Dujon, et al. (1996). “Two different but related mechanisms are used in plants for the repair of genomic double-strand breaks by homologous recombination.” Proc Natl Acad Sci U S A 93(10): 5055-60.
Rosen, L. E., H. A. Morrison, et al. (2006). “Homing endonuclease I-CreI derivatives with novel DNA target specificities.” Nucleic Acids Res.
Rothstein, R. (1991). “Targeting, disruption, replacement, and allele rescue: integrative DNA transformation in yeast.” Methods Enzymol 194: 281-301.
Rouet, P., F. Smih, et al. (1994). “Expression of a site-specific endonuclease stimulates homologous recombination in mammalian cells.” Proc Natl Acad Sci USA 91(13): 6064-8.
Rouet, P., F. Smih, et al. (1994). “Introduction of double-strand breaks into the genome of mouse cells by expression of a rare-cutting endonuclease.” Mol Cell Biol 14(12): 8096-106.
Sargent, R. G., M. A. Brenneman, et al. (1997). “Repair of site-specific double-strand breaks in a mammalian chromosome by homologous and illegitimate recombination.” Mol Cell Biol 17(1): 267-77.
Seligman, L. M., K. M. Stephens, et al. (1997). “Genetic analysis of the Chlamydomonas reinhardtii I-Crel mobile intron homing system in Escherichia coli.” Genetics 147(4): 1653-64.
Siebert, R. and H. Puchta (2002). “Efficient Repair of Genomic Double-Strand Breaks by Homologous Recombination between Directly Repeated Sequences in the Plant Genome.” Plant Cell 14(5): 1121-31.
Silva, G. H., J. Z. Dalgaard, et al. (1999). “Crystal structure of the thermostable archaeal intron-encoded endonuclease I-DmoI.” J Mol Biol 286(4): 1123-36.
Simon, P., F. Cannata, et al. (2008). “Sequence-specific DNA cleavage mediated by bipyridine polyamide conjugates.” Nucleic Acids Res 36(11): 3531-8.
Smith, J., S. Grizot, et al. (2006). “A combinatorial approach to create artificial homing endonucleases cleaving chosen sequences.” Nucleic Acids Res 34(22): e149.
Sonoda, E., H. Hochegger, et al. (2006). “Differential usage of non-homologous end-joining and homologous recombination in double strand break repair.” DNA Repair (Amst) 5(9-10): 1021-9.
Spiegel, P. C., B. Chevalier, et al. (2006). “The structure of I-CeuI homing endonuclease: Evolving asymmetric DNA recognition from a symmetric protein scaffold.” Structure 14(5): 869-80.
Stoddard, B. L. (2005). “Homing endonuclease structure and function.” Q Rev Biophys 38(1): 49-95.
Sussman, D., M. Chadsey, et al. (2004). “Isolation and characterization of new homing endonuclease specificities at individual target site positions.” J Mol Biol 342(1): 31-41.
Teicher, B. A. (2008). “Next generation topoisomerase I inhibitors: Rationale and biomarker strategies.” Biochem Pharmacol 75(6): 1262-71.
Terada, R., Y. Johzuka-Hisatomi, et al. (2007). “Gene targeting by homologous recombination as a biotechnological tool for rice functional genomics.” Plant Physiol 144(2): 846-56.
Terada, R., H. Urawa, et al. (2002). “Efficient gene targeting by homologous recombination in rice.”Nat Biotechnol 20(10): 1030-4.
Wang, R., X. Zhou, et al. (2003). “Chemically regulated expression systems and their applications in transgenic plants.” Transgenic Res 12(5): 529-40.
Zuo, J. and N. H. Chua (2000). “Chemical-inducible systems for regulated expression of plant genes.” Curr Opin Biotechnol 11(2): 146-51.
Grizot, S., J. C. Epinat, et al. (2009). “Generation of redesigned homing endonucleases comprising DNA-binding domains derived from two different scaffolds.” Nucleic Acids Res 38(6): 2006-18.
Mazur, D. J. and F. W. Perrino (2001). “Structure and expression of the TREX1 and TREX2 3′-->5′ exonuclease genes.” J Biol Chem 276(18): 14718-27.
Perrino, F. W., de Silva U, Harvey S, Pryor E. E. Jr., Cole D. W. and Hollis T (2008). “Cooperative DNA binding and communication across the dimer interface in the TREX2 3′-->5′-exonuclease.” J Biol Chem 283 (31): 21441-52.

1-51. (canceled)

52. A method for increasing double-strand break induced mutagenesis at a genomic locus of interest in a cell comprising the steps of:
(i) identifying at said genomic locus of interest at least one DNA target sequence cleavable by one rare-cutting endonuclease;
(ii) engineering said at least one rare-cutting endonuclease in order to generate a loss of genetic information around said DNA target sequence within the genomic locus of interest; and
(iii) contacting said DNA target sequence with said at least one rare-cutting endonuclease to generate said loss of genetic information around said DNA target sequence within the genomic locus of interest;
thereby obtaining a cell in which double-strand break induced mutagenesis at said genomic locus of interest is increased.

53. The method according to claim 52, wherein said engineered rare-cutting endonuclease is a chimeric rare-cutting endonuclease comprising a catalytic domain selected from table 2 (SEQ ID NO: 38-57) and table 3 (SEQ ID NO: 96-152), a functional mutant, a variant or a derivative thereof.

54. The method according to claim 53, wherein said chimeric rare-cutting endonuclease comprises a catalytic domain selected from the group of Trex (SEQ ID NO: 145-149) and Tdt (SEQ ID NO: 201), a functional mutant, a variant or a derivative thereof.

55. The method according to claim 54, wherein said chimeric rare-cutting endonuclease comprises a catalytic domain of SEQ ID NO: 194, a functional mutant, a variant or a derivative thereof.

56. The method according to claim 52, comprising the steps of:
(i) identifying at said genomic locus of interest one DNA target sequence cleavable by one rare-cutting endonuclease;
(ii) engineering said at least one rare-cutting endonuclease such that said rare-cutting endonuclease is able to generate at least two nearby DNA double-strand breaks in the genomic locus of interest;
(iii) contacting said DNA target sequence with said at least one rare-cutting endonuclease;
thereby obtaining a cell in which double-strand break induced mutagenesis at said genomic locus of interest is increased.

57. The method according to claim 52, comprising the steps of:
(i) identifying at said genomic locus of interest two nearby DNA target sequences respectively cleavable by one rare-cutting endonuclease;
(ii) engineering a first rare-cutting endonuclease able to generate a first DNA double-strand break in the genomic locus of interest;
(iii) engineering a second rare-cutting endonuclease able to generate a second DNA double-strand break in the genomic locus of interest;
(iv) contacting said DNA target sequence with said two rare-cutting endonucleases; thereby obtaining a cell in which double-strand break induced mutagenesis at said genomic locus of interest is increased.

58. The method according to claim 56, wherein said at least two nearby DNA double-strand breaks into said genomic locus of interest are distant between 12 bp and 200 bp.

59. The method according to claim 56, wherein said rare-cutting endonuclease able to generate at least two nearby DNA double-strand breaks into a genomic locus of interest is a chimeric rare-cutting endonuclease comprising at least two catalytic domains.

60. The method according to claim 59, wherein said chimeric rare-cutting endonuclease is a fusion protein between a meganuclease and at least one nuclease catalytic domain.

61. The method according to claim 60, wherein said nuclease catalytic domain has endonuclease activity.

62. The method according to claim 61, wherein said nuclease catalytic domain is selected from table 2 (SEQ ID NO: 38-57) and table 3 (SEQ ID NO: 96-152), Col E7 (SEQ ID NO: 97), I-Tev I (SEQ ID NO: 106 or SEQ ID NO: 60; SEQ ID NO: 107-108), NucA (SEQ ID NO: 41 and 1 12), NucM (SEQ ID NO: 43 and 113), SNase (SEQ ID NO: 45-47 and 116-118) functional mutants, variants or derivatives thereof.

63. The method according to claim 60, wherein said nuclease catalytic domain has an exonuclease activity.

64. The method according to claim 59, wherein said chimeric rare-cutting endonuclease is a fusion protein between a meganuclease, one nuclease catalytic domain and one other catalytic domain.

65. The method according to claim 59, wherein said meganuclease and said nuclease catalytic domain are bound by at least a peptidic linker.

66. A chimeric rare-cutting endonuclease to generate at least two nearby DNA double-strand breaks in a genomic locus of interest comprising:
i) a rare-cutting endonuclease;
ii) a peptidic linker; and
iii) a nuclease catalytic domain.

67. A chimeric rare-cutting endonuclease according to claim 66, further comprising:
i) a second peptidic linker,
ii) a supplementary catalytic domain, or
iii) a second peptidic linker and a supplementary catalytic domain.

68. A chimeric rare-cutting endonuclease according to claim 67, wherein said supplementary catalytic domain has a nuclease activity.

69. A recombinant polynucleotide encoding a chimeric rare-cutting endonuclease according to claim 66.

70. A vector comprising a recombinant polynucleotide according to claim 69.

71. A composition comprising a chimeric rare-cutting endonuclease according to claim 66 and a carrier.

72. A kit comprising a chimeric rare-cutting endonuclease according to claim 66 and instructions for use in increasing double-strand break-induced mutagenesis in a eukaryotic cell and optionally packaging materials, containers for the ingredients, and other components used for increasing double-strand break-induced mutagenesis.

73. A method for increasing double-strand break induced mutagenesis at a genomic locus of interest in a cell comprising the steps of:
(i) identifying at said genomic locus of interest one DNA target sequence cleavable by one rare-cutting endonuclease nearby one DNA target sequence cleavable by one frequent-cutting endonuclease;
(ii) engineering said rare-cutting endonuclease such that said rare-cutting endonuclease is able to generate one DNA double-strand break in the genomic locus of interest;
(iii) making a fusion protein between said rare-cutting endonuclease and said frequent-cutting endonuclease;
(iv) contacting said DNA target sequences with said fusion protein to generate at least two nearby double-strand breaks;
thereby obtaining a cell in which double-strand break induced mutagenesis at said genomic locus of interest is increased.

74. A fusion protein to generate at least two nearby DNA double-strand breaks into a genomic locus of interest comprising:
i) a rare-cutting endonuclease;
ii) a peptidic linker; and
ii) a frequent-cutting endonuclease.

75. A fusion protein according to claim 74, further comprising:
i) a second peptidic linker,
ii) a supplementary catalytic domain, or
iii) a second peptidic linker and a supplementary catalytic domain.

76. A fusion protein according to claim 75, wherein said supplementary catalytic domain has a nuclease activity.

77. An isolated, purified or recombinant polynucleotide encoding a fusion protein according to claim 74.

78. A vector comprising the polynucleotide according to claim 77.

79. A composition comprising a fusion protein according to claim 74 and a carrier.

80. A kit comprising a fusion protein according to claim 74, and instructions for use in increasing double-strand break-induced mutagenesis in a cell and optionally packaging materials, containers for the ingredients, and other components used for increasing double-strand break-induced mutagenesis.
